(19) United States
(12) Patent Application Publication
(10) Pub. No.: US 2005/0192754 A1
(43) Pub. Date: Sep. 1, 2005

(54) Protein sequence signals and their applications

(75) Inventor: Cornelius G. Hunter, Cameron Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Seagull Technology, Inc., Campbell, CA (US)

(21) Appl. No.: 10/788,898

(22) Filed: Feb. 26, 2004

(51) Int. Cl. G06F 19/00; G01N 33/48; G01N 33/50

(52) U.S. Cl. 702/19

(57) ABSTRACT

Just as written languages appear random unless one knows the words, so too protein sequences can appear as random. By statistical measures they are far from random. Protein sequences contain nonrandom signals. Some signals are associated with structure and function. Methods to search for and identify such signals are provided. Two amino acid classes and the characteristics of their signals are described. Protein sequences are transformed into symbols using these classes and other sets of amino acids. Signals are identified from these symbols. Signal analysis has many applications. As an example, conserved signal patterns across different protein families are used to predict fold of query sequences.

### BACKGROUND OF THE INVENTION

Proteins consist of amino acids linked together in a sequence, and the amino acid sequence is typically sufficient to specify the protein structure and function. Protein sequences have been studied for forty years yet they have defied systematic description of their information content which might indicate how the structure and function are specified. It is not surprising that protein sequences have been described as practically random.

Existing methods of analyzing proteins are performed at the level of primary amino acid sequence and are not sufficient to predict protein structure. It is possible to compare an amino acid sequence to a database of sequences to identify conserved regions of proteins. An example of such a method is the basic local alignment sequence tool, known as BLAST, which is available through the National Center for Biotechnology Information. Using a query protein sequence as the input and comparing the sequence to databases of either known protein sequences or translated nucleotide sequences typically yields a list of sequences identified as having a certain degree of amino acid sequence identity with the query sequence. It is typical that some sections of a query amino acid sequence display a significant level of sequence identity with certain sections of other proteins but no amino acid sequence identity to other sequences in other sections of the query sequence.

Predicting the structure of a protein based on amino acid sequence has been a goal of protein chemists and molecular biologists for decades. The most common methodology for performing such predictions revolves around comparing a query protein sequence to a database of known sequences and selecting a protein with a similar sequence for which the protein structure is already known. The query sequence is then “threaded” into the known structure and a series of energy minimization algorithms are used to allow the hypothetical structure to adopt a slightly different conformation based on amino acid sequence differences between the two proteins. For example, if the sequence of known structure has an alanine residue in a certain position while the query sequence has an isoleucine residue at the comparable position, the threaded structure will be allowed freedom to adjust the local structural environment to make room for the additional atoms in the isoleucine residue. The main problem with threading methodology is that the proposed structure is highly biased by the preexisting known structure. In other words, threading methods assume that the query amino acid sequence would have adopted the fold of the preexisting known structure and then adapted its local environments to adjust for variations in side chain identity. The less overall sequence identity that exists between a query sequence and a protein of known structure, the more speculative these types of modeling protocols become, magnifying the bias of the preexisting known structure.

To avoid such bias, it is desirable to make structural predictions of proteins based only on amino acid sequence. Without relying on the known propensity of certain amino acid motifs to form certain secondary structures, modeling protein structure based purely on the chemical properties of amino acids is not a straightforward task either. Relying on the known propensity of certain amino acid motifs to form certain secondary structures is to some degree desirable. However, problems arise from the fact that it is not clear where propensity information stops being predictive and starts being misleading in terms of introducing excess structural bias into modeling calculations.

Designing proteins with a known function is a highly desirable goal. However, this task is complicated by the astronomical number of protein sequences which are theoretically possible. For example, a 100 residue protein has 20100 possible sequences. Rather than attempting to design proteins with novel functions de novo, two basic approaches have traditionally been used. One approach involves site directed mutagenesis of proteins with known structure and function. The other involves random combinatorial mutagenesis of proteins with known function. Both of these methods rely on preexisting tertiary structure to be retained. A third approach to designing proteins with novel functions involves screening of completely random peptide sequences, such as phage display methods. Completely random methods such as phage display are useful for obtaining peptide sequences that bind to certain target structures, but are generally not powerful enough to generate peptides with novel catalytic activities. The main reason for this is that to possess a buried active site cavity capable of catalyzing a biochemical reaction, a protein sequence must be over a certain size, such as 50 amino acids or more. Out of the total possible number of sequences that can be created in a random 50 amino acid protein (20⁵⁰), only a small number will actually fold into a globular structure that would be capable of housing an active site cavity. The result is that proteins created with novel functional properties must be either (a) small and not capable of catalytic activity or (b) highly similar in overall structure to preexisting proteins.

Another computational method used in bioinformatics is the identification of protein sequences within sections of nucleotide sequence. It is relatively straightforward to predict open reading frames within short nucleotide sequences such as 1 kB by searching for start codons (ATG) followed downstream by in-frame stop codons (TAA, TAG, and TGA). Searching a nucleotide sequence for the protein coding sequence requires searching only the 5′ to 3′ direction in each reading frame. To completely search a nucleotide sequence for open reading frames, the sequence must be searched in all three reading frames on both strands. The process becomes significantly more complicated when the sequence being searched is a relatively long (10 kB or more) stretch of genomic DNA, particularly if it is from a eukaryotic organism. Because eukaryotic genes usually have introns, the start and stop codons for a gene may be tens or even hundreds of kB apart. Most of the sequence between them is non-coding intron sequence, and it is not always a simple task to elucidate the exon-intron boundaries between the start and stop codons using purely computational methods. As an example, it was considered relatively surprising that upon completion of the human genome project, only an estimated 40,000 genes were found in the human genome sequence. cDNA library data, however, suggests that the number of genes in the human genome might be significantly higher. This discrepancy may have to do with the limitations of the software and methods used to identify the 40,000 genes across a 3 billion base pair genome.

### BRIEF SUMMARY OF THE INVENTION

Methods are provided for analyzing a sequence of amino acids, comprising (a) designating each amino acid within the sequence with a symbol, wherein an amino acid is designated a first symbol if it is a member of a predetermined set of amino acids, and a second symbol different from the first symbol if the amino acid is not a member of the predetermined set, thereby producing a sequence of symbols; and (b) determining which signals of the symbols are present in the sequence of symbols, wherein a signal is a window of the sequence of symbols consisting of a predefined number of contiguous symbols, wherein the sequence of amino acids is analyzed from the identity of the signals present in the sequence of symbols. In some methods the window consists of 5-15 contiguous symbols, more preferably 9 contiguous symbols. Some methods further comprising providing user input of the predefined number of contiguous symbols of the window. Some methods further comprising repeating steps (a) and (b) for a second sequence of amino acids and aligning the sequences of symbols produced from the first and second sequences of amino acids for maximum conservation of significant signals. In some methods step (b) determines the identity of L-(P-1) signals within the sequence of amino acids, where L is length of the sequence of amino acids and P is the predefined number of contiguous symbols in the window. Some methods further comprise inputting the sequence of amino acids into the computer. In other methods the sequence of amino acids is input by transfer of data from a database. Some methods further comprising outputting the identity of signals present in the sequence of symbols. In some methods the signals are output in an order corresponding to the order of amino acids in the sequence of amino acids.

In some methods the predetermined set of amino acids consists of 4-10 amino acids, and at least 4 are selected from the group consisting of A, R, Q, E, L, K and M. In some methods the set of amino acids consists of A, R, Q, E, L, K and M. In some methods the predetermined set of amino acids consists of 4-10 amino acids, and at least 4 are selected from the group consisting of C, I, L, M, F, W, Y, and V. In some methods the predetermined set of amino acids consists of C, I, L, M, F, W, Y, and V.

Some methods further comprise transforming the sequence of symbols into a sequence of signal designations, wherein different designations are used to represent different signals in the sequence of symbols.

In some methods an amino acid is designated with a first type of second symbol if it is part of a second predetermined set of amino acids, and a second type of second symbol if it is not part of the second set of amino acids.

In some methods the signals present in the sequence of symbols are assigned grades according to the probability that the observed frequency of a signal in a collection of proteins in which each amino acid has been designated with a symbol occurs by chance, wherein the grade increases with decreasing probability. In some methods the signals are classified as significant or not significant signals depending whether the grade exceeds a threshold. In some methods the threshold is a χ²>8 that the observed frequency of the signal in the collection of proteins does not occur by chance. Some methods further comprise determining the number and identity of significant signals in the amino acid sequence.

In some methods at least one signal present in the sequence of symbols is present in Table 14. In some methods at least one signal present in the sequence of symbols is present in Table 14.

In some methods the sequence of amino acids to be analyzed is a theoretical amino acid sequence, and the method comprises determining the probability that the theoretical amino acid sequence is an actual protein by comparing the expected number of significant signals in the theoretical amino acid sequence to the actual number of significant signals in the theoretical amino acid sequence. In some methods the theoretical amino acid sequence is designated as an actual protein sequence if the probability that the observed significant signals in the sequence arose by chance is 10⁻¹⁰ or less. In some methods the sequence of amino acids is from a known protein. In other methods the sequence of amino acids is from a putative protein.

Some methods comprising predicting the secondary structure of a segment of a protein located within the sequence of amino acids from the identity of significant signals. In some methods the secondary structure is selected from the group consisting of an alpha helix, beta strand, beta turn, turn+beta, helix+turn, helix cap, extended helix, Gly/Pro twist, beta+turn, helix-hairpin, beta cap, helix hairpin, beta hairpin, contorted helix, turn, helix+turn II and helix turn.

Some methods comprise calculating the probability that the observed frequency of a signal in a collection of proteins in which each amino acid has been designated with a symbol occurs by chance.

Some methods comprise comparing the position and identity of each signal present in a sequence of symbols to a conserved signal pattern present in a family of proteins.

Some methods comprise assigning the determined signals designations, a different designation being used for each unique signal. Some methods comprise analyzing the sequence of amino acids from the identity of the signals.

Also provided are computer implemented methods of identifying a set of amino acids useful for the analysis of proteins, comprising (a) designating each amino acid within each of a collection of proteins with a symbol, wherein an amino acid is designated a first symbol if it is a member of a first test set of amino acids, and a second symbol different from the first symbol if the amino acid is not a member of the test set, thereby producing a collection of sequences of symbols; (b) determining the number of occurrences of different signals of the symbols in the collection of sequences of symbols, wherein a signal is a window of the sequence of symbols consisting of a predefined number of contiguous symbols; and (c) determining the probability that the distribution of the number of signals of each signal strength occurs by chance, wherein the lower the probability the more useful the test set of amino acids is for protein analysis. Some methods further comprise repeating steps (a), (b) and (c) for a second test set of amino acids. In some methods the second test set differs from the first test set by the addition, deletion, or substitution of an amino acid from the first test set. Some methods further comprise repeating steps (a), (b) and (c) for each possible unique set of amino acids consisting of 4-10 amino acids.

Also provided are computer-implemented methods of predicting the fold of a query protein comprising; (a) designating each amino acid within a family of protein sequences with a symbol, wherein an amino acid is designated a first symbol if it is a member of a predetermined set of amino acids, and a second symbol different from the first symbol if the amino acid is not a member of the set, thereby producing a plurality of sequences of symbols; (b) determining which signals of the symbols are present in the sequences of symbols, wherein a signal is a window of the sequence of symbols consisting of a predefined number of contiguous symbols; (c) determining a conserved signal pattern between members of the family; (d) analyzing a query protein to identify a signal pattern; (e) determining if the query protein's signal pattern exceeds a threshold of similarity to the conserved signal pattern; and (f) if the signal pattern of the query exceeds the threshold, designating the query as having the fold of the family. Some methods further comprise comparing the query protein's signal pattern to conserved signal patterns in an additional protein family. In some methods the family is selected from the list consisting of globins, lysozymes, thioredoxins, trypsins, monoclonal antibodies, and amido transferases. In some methods the conserved signal pattern includes a signal present in Table 14. In some methods the conserved signal pattern includes a signal present in Table 15.

Also provided are computer program products for analyzing a sequence of amino acids, comprising (a) code for designating each amino acid within the sequence with a symbol, wherein an amino acid is designated a first symbol if it is a member of a predetermined set of amino acids, and a second symbol different from the first symbol if the amino acid is not a member of the set, thereby producing a sequence of symbols; (b) code for determining which signals of the symbols are present in the sequence of symbols, wherein a signal is a window of the sequence of symbols consisting of a predefined number of contiguous symbols, wherein the sequence of amino acids is analyzed from the identity of the signals present in the sequence of symbols; and (c) a computer readable storage medium holding the codes.

Also provided are computer program products for identifying a set of amino acids useful for the analysis of proteins, comprising (a) code for designating each amino acid within each of a collection of proteins with a symbol, wherein an amino acid is designated a first symbol if it is a member of a first test set of amino acids, and a second symbol different from the first symbol if the amino acid is not a member of the test set, thereby producing a collection of sequences of symbols; (b) code for determining the number of occurrences of different signals of the symbols in the collection of sequences of symbols, wherein a signal is a window of the sequence of symbols consisting of a predefined number of contiguous symbols; (c) code for determining the probability that the distribution of the number of signals of each signal strength occurs by chance, wherein the lower the probability the more useful the test set of amino acids is for protein analysis; and (d) a computer readable storage medium holding the codes.

Also provided are computer program products for predicting the fold of a query protein comprising (a) code for designating each amino acid within a family of protein sequences with a symbol, wherein an amino acid is designated a first symbol if it is a member of a predetermined set of amino acids, and a second symbol different from the first symbol if the amino acid is not a member of the set, thereby producing a plurality of sequences of symbols; (b) code for determining which signals of the symbols are present in the sequences of symbols, wherein a signal is a window of the sequence of symbols consisting of a predefined number of contiguous symbols; (c) code for determining a conserved signal pattern between members of the family; (d) code for analyzing a query protein to identify a signal pattern; (e) code for determining if the query protein's signal pattern exceeds a threshold of similarity to the conserved signal pattern; and (f) code for designating the query as having the fold of the family if the signal pattern of the query exceeds the threshold.

Also provided are computer program products for identifying a coding region of a nucleotide sequence comprising (a) code for translating all possible reading frames of a nucleotide sequence into theoretical protein sequences; (b) code for designating each amino acid within the theoretical protein sequences with a symbol, wherein an amino acid is designated a first symbol if it is a member of a first predetermined set of amino acids, and a second symbol different from the first symbol if the amino acid is not a member of the predetermined set, thereby producing a collection of sequences of symbols; (c) code for determining the number of significant signals in each reading frame of the nucleotide sequence; and (d) code for determining an expected number of significant signals in each reading frame of the nucleotide sequence.

Also provided are systems for analyzing a sequence of amino acids, comprising:
a memory; (b) a system bus; and (c) a processor operatively disposed to (i) designate each amino acid within the sequence with a symbol, wherein an amino acid is designated a first symbol if it is a member of a predetermined set of amino acids, and a second symbol different from the first symbol if the amino acid is not a member of the set, thereby producing a sequence of symbols; (ii) determine which signals of the symbols are present in the sequence of symbols, wherein a signal is a window of the sequence of symbols consisting of a predefined number of contiguous symbols, wherein the sequence of amino acids is analyzed from the identity of the signals present in the sequence of symbols.

Also provided are systems for identifying a set of amino acids useful for the analysis of proteins comprising (a) a memory; (b) a system bus; and (c) a processor operatively disposed to (i) designate each amino acid within each of a collection of proteins with a symbol, wherein an amino acid is designated a first symbol if it is a member of a first test set of amino acids, and a second symbol different from the first symbol if the amino acid is not a member of the test set, thereby producing a collection of sequences of symbols; (ii) determine the number of occurrences of different signals of the symbols in the collection of sequences of symbols, wherein a signal is a window of the sequence of symbols consisting of a predefined number of contiguous symbols; and (iii) determine the probability that the distribution of the number of signals of each signal strength occurs by chance, wherein the lower the probability the more useful the test set of amino acids is for protein analysis.

Also provided are systems for predicting the fold of a query protein comprising (a) a memory; (b) a system bus; and (c) a processor operatively disposed to (i) designate each amino acid within a family of protein sequences with a symbol, wherein an amino acid is designated a first symbol if it is a member of a predetermined set of amino acids, and a second symbol different from the first symbol if the amino acid is not a member of the set, thereby producing a plurality of sequences of symbols (ii) determine which signals of the symbols are present in the sequences of symbols, wherein a signal is a window of the sequence of symbols consisting of a predefined number of contiguous symbols; (iii) determine a conserved signal pattern between members of the family; (iv) analyze a query protein to identify a signal pattern; (v) determine if the query protein's signal pattern exceeds a threshold of similarity to the conserved signal pattern; and (vi) designate the query as having the fold of the family if the signal pattern of the query exceeds the threshold.

Also provided are systems for identifying a coding region of a nucleotide sequence comprising (a) a memory; (b) a system bus; and (c) a processor operatively disposed to (i) translate all possible reading frames of a nucleotide sequence into theoretical protein sequences; (ii) designate each amino acid within the theoretical protein sequences with a symbol, wherein an amino acid is designated a first symbol if it is a member of a first predetermined set of amino acids, and a second symbol different from the first symbol if the amino acid is not a member of the predetermined set, thereby producing a collection of sequences of symbols; (iii) determine the number of significant signals in each reading frame of the nucleotide sequence; and (iv) determine an expected number of significant signals in each reading frame of the nucleotide sequence.

### BRIEF DESCRIPTION OF THE DRAWINGS

FIG. 1: Signal strength distribution of class 1 signals for a window of 9 residues. The class 1 signal amino acids tend to cluster relative to the random distribution. The deviation from the random distribution has a χ² value of 3985, which is equivalent to a probability of 10⁻⁸⁵⁶ of being the result of random sequences.

FIG. 2: Signal strength distribution of class 2 signals for a window of 9 residues. The class 2 signal amino acids tend to anticluster relative to the random distribution. The deviation from the random distribution has a χ² value of 5173, which is equivalent to a probability of 10⁻¹¹¹⁴ of being the result of random sequences.

FIG. 3: Class 1 signal properties. (A) Signals with increasing sequence χ², and therefore increasing statistical significance, have increasing correlation with local structure, as indicated by the local structure χ². Signals with high statistical significance may have high helix or strand propensity and these two groups form different traces. (B) The different traces are labeled according to their signal strength. Signal strength extremes (0-1,7-9) have high sequence χ² and super unity frequency. (C) A signal frequency minima occurs at medium signal strength values. (D) Local structure is strongly correlated with signal strength. Helix propensity is proportional to signal strength and strand propensity is inversely proportional to signal strength.

FIG. 4: Class 2 signal properties. (A) Local structure χ² is correlated with sequence χ². (B) The different traces are labeled according to their signal strength. High signal strength (6-9) has low sequence χ² and sub unity frequency. (C) A signal frequency maxima occurs at medium signal strength values. (D) Helix propensity is weak at low signal strength.

FIG. 5: A sample result for protein sequence segments eight residues in length. The randomized test case shows that only a negligible number of signals are expected to have sequence χ² values greater than 8. The actual sequence data contains hundreds of class 2 sequence signals with sequence χ² values ranging from 8 to 1000.

FIG. 6: Histogram of distribution of signal 92 (000100100, generated by class 2 amino acids) in the globin family and fitted signal location probability density function. The abscissa progresses from the N-to-C terminii of the sequence.

FIG. 7: Histogram of distribution of Signal 92 (000100100, generated by class 2 amino acids) in the thioredoxin family and fitted signal location probability density function. The abscissa progresses from the N-to-C terminii of the sequence.

FIG. 8: Comparison of signal 92 (000100100, generated by class 2 amino acids) normalized probability distribution functions in the globin (thin line) and thioredoxin (thick line) families. The plots illustrate that signal location is a powerful discriminator in protein-protein comparisons.

FIG. 9: Distribution of class 1 training set scores for both the fold which the sequence codes for (identity) and the other folds in the database (competing). The majority of the class 1 identity scores are several orders of magnitude greater than the class 1 competing scores.

FIG. 10: Distribution of class 2 training set scores for both the fold which the sequence codes for (identity) and the other folds in the database (competing). The majority of the class 2 identity scores are several orders of magnitude greater than the class 2 competing scores.

FIG. 11: Distribution of the ratio of the class 1 training set identity score to the respective highest competing score. The majority of the class 1 identity predictions are several orders of magnitude greater than the nearest competing score.

FIG. 12: Distribution of the ratio of the class 2 training set identity score to the respective highest competing score. The majority of the class 2 identity predictions are several orders of magnitude greater than the nearest competing score.

FIG. 13: Schematic depiction of a suitable computer system for performing the described methods.

FIG. 14: Depiction of a suitable computer system for performing the described methods.

FIG. 15: Flow chart depicting simplified steps in analyzing a sequence of amino acids.

FIG. 16: Flow chart depicting simplified steps in identifying a set of amino acids useful for analysis of proteins.

FIG. 17: Flow chart depicting simplified steps in predicting the fold of a query protein.

FIG. 18: Flow chart depicting steps in determining useful sets of amino acids.

FIG. 19: Flow chart depicting steps in transforming amino acid sequences into sequences of signal designations.

FIG. 20: Flow chart depicting steps in scanning nucleotide sequences for protein coding regions.

FIG. 21: Flow chart depicting steps in predicting the fold of a query protein.

### DETAILED DESCRIPTION OF THE INVENTION DEFINITIONS

Conserved signal pattern: The recurrence, at a frequency above what is expected by chance, of one or more specific signals at similar locations within two or more members of a protein family.

Fold: The three dimensional structure of a protein's backbone, as defined by the relative three dimensional relationships between elements of secondary structure. The “backbone” refers to the peptide bond chain of the amino acid sequence and does not include side chains. The fold of a protein can include elements of secondary structure such as alpha helices, beta sheets, and turns. The primary structure of a protein, which is simply its amino acid sequence, dictates the fold of a protein.

Helix propensity: The propensity of a peptide of a particular sequence to adopt a helical shape.

Local structure centroid: Canonical structural fragments of proteins such as an alpha helix, beta strand, beta turn, turn+beta, helix+turn, helix cap, extended helix, Gly/Pro twist, beta+turn, helix-hairpin, beta cap, helix hairpin, beta hairpin, contorted helix, turn, helix+turn II, and helix turn (as discussed in Hunter, C. G. and Subramaniam, S. “Protein Fragment Clustering and Canonical Local Shapes,” Proteins: Struct., Funct. and Gen., 50: 580-588, 2003).

Local structure χ² value: A measurement of the observed occurrences of local structure centroids along a protein backbone where a signal occurs compared to the randomly expected number of occurrences of local structure centroids where a signal occurs.

Protein family: A collection of proteins that arose from a common evolutionary sequence and have the same fold. Preferably, a family of sequences used for fold analysis contains sequences that each have at least 20% amino acid sequence identity with all other members of the family but no more than 90% amino acid sequence identity with any member of the family.

“Protein sequence”, “peptide sequence”, and “amino acid sequence” refer to the sequence of amino acids in a linear polypeptide chain that can be actual or putative.

Putative protein: A theoretical or hypothetical protein. A hypothetical protein sequence does not occur naturally and is designed for the purpose of carrying out a desired function. A theoretical protein is an amino acid sequence that arises from translating a nucleotide sequence in a particular reading frame.

Sequence χ²: The probability that an observed signal pattern, distribution of signals, frequency of occurrence of a particular signal, and other measurements of signals is a random event.

Set of amino acids: A “set” of amino acids means a set of 2-19 of the 20 naturally occurring amino acids. A “test set” of amino acids means a set of amino acids that is tested for usefulness in transforming an amino acid sequence into a sequence of symbols defining signals. A test set is useful if the distribution of signal strengths in a collection of transformed amino acid sequences (eg., the collection of Table 3) occurs at a frequency significantly different than occurs by chance (eg., a probability of <10⁻¹⁰⁰, or more preferably a probability of <10⁻⁵⁰⁰). When a test set that has been determined to be useful is subsequently used to transform an amino acid sequence of interest, the test set is referred to as a “predetermined set.”

Signal designation: An arbitrary symbol used to represent a particular signal.

Signal frequency: The observed number of occurrences of a signal in a collection of protein sequences. The signal frequency may be sub or super unity with regard to their sequence χ². Signals of high probability, for example, may have low or high frequencies.

Signal grade: Signals are assigned grades depending on the signal's sequence χ² or other probability measurement. For example, the grade can be “significant” if the probability that the detected signal is not a chance occurrence exceeds a specified threshold, and “not significant” if the probability is below the threshold. A “significant signal” occurs in a collection of protein sequences at a frequency higher or lower than expected by chance.

Signal location probability density function (PDF): The probability that a signal appears at a certain point relative to the beginning and end of a given protein.

Signal pattern: The sequence of signals generated by transforming a sequence of amino acids into a sequence of symbols using a set of amino acids and a given window length.

Signal strength distribution: The distribution of all signals of a given signal strength for signals generated using a given test set within a collection of proteins.

Signal sequence χ² value: A measurement of the difference between how often a signal is expected to occur in a collection of proteins by chance and the actual occurrence of that signal in a collection of proteins.

Signal strength (Nₛₛ): The number of amino acids of a test set of amino acids in a given signal. Note that a weak signal, for example, may have high probability of occurring in a protein sequence and a high actual frequency of occurring.

Signal: A sequence of symbols depicting the amino acids of a set of amino acids in a given sequence window. Signals are generated by transforming an amino acid sequence into symbols according to a set of amino acids and designating a window length. There are L-(P-1) signals per amino acid sequence, where L is the length of the amino acid sequence and P is the predefined number of contiguous symbols in a window.

Strand propensity: The propensity of a peptide of a particular sequence to adopt a beta strand shape.

Symbol: A designation of an amino acid that identifies the amino acid as inside or outside a set of amino acids.

Transformed amino acid sequence: A sequence of symbols that results from assigning a first symbol to all amino acids in the sequence that fall into a set of amino acids and a second symbol to all amino acids that do not fall into the set of amino acids.

“Window” or “sequence window”: A predefined number of contiguous symbols representing amino acids that are analyzed within a sequence of symbols. The length of the window is designated as N_{w}. The window can be moved through a sequence of symbols to provide separate “views” of each contiguous stretch of symbols corresponding to the length of the window. For example, a 9 symbol window views each 9 symbol segment of a protein. The first segment the windows views is amino acids 1-9, the second segment viewed is amino acids 2-10, and so on. By moving the window through the entire length of the sequence of symbols, each contiguous stretch of 9 symbols is viewed individually. Overlapping signals contain at least one symbol that was generated by the same amino acid in the protein sequence, such as signals that are generated by amino acids 1-9 and 3-11 of a transformed amino acid sequence. Non-overlapping signals do not contain any symbols that were generated by the same amino acid in the protein sequence.

Conventional alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

Another example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra.). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For identifying whether a nucleic acid or polypeptide is within the scope hereof, the default parameters of the BLAST programs are suitable. The BLASTN program (for nucleotide sequences) uses as defaults a word length (W) of 11, an expectation (E) of 10, M=5, N=4, and a comparison of both strands. For amino acid sequences, the BLASTP program uses as defaults a word length (W) of 3, an expectation (E) of 10, and the BLOSUM62 scoring matrix. The TBLASTN program (using protein sequence for nucleotide sequence) uses as defaults a word length (W) of 3, an expectation (E) of 10, and a BLOSUM 62 scoring matrix. (see Henikoff& Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

I. General

The invention provides methods of analyzing a sequence of amino acids in which the sequence of amino acids is first transformed into a series of a symbols. An amino acid is designated a first symbol if it is a member of a predetermined set of amino acids, and a second symbol if it not. For example, the predetermined set of amino acids can be a set of hydrophobic amino acids consisting of C, I, L, M, F, W, Y and V, and the first and second symbols can be 1 and 0, thereby generating a binary code of 1's and 0's. The sequence of symbols is then analyzed to determine which signals are present within it. A signal is a pattern of symbols depicting the amino acids of a given set in a given sequence window. For example, for a window of 9 contiguous symbols, there are 29 possible signals. Examples of such signals include 100111000 and 010101111. The sequence of symbols is analyzed to determine which signals are present. For example, signals occupying a window of 9 amino acids are analyzed. Analysis is performed by first examining the signals corresponding to the symbols generated by amino acids 1-9 in the protein sequence. Next, the symbols generated by amino acids 2-10 are analyzed, followed the symbols generated by amino acids 3-11, and so on. All signals are generated using a specific set of amino acids. This means that the same amino acid sequence generates different signals depending on which predetermined set of amino acids is used to transform the amino acid sequence.

Within a given amino acid sequence, usually only a subset of all possible signals is present. The signals that are present allow analysis of the amino acid sequence in a variety of ways. For example, the signals can be classified as significant or not significant depending on whether a signal occurs at a significantly different frequency than would be expected based on random distribution of amino acids in a collection of protein sequences. Natural proteins contain many more significant signals than do randomly generated sequences of amino acids. Therefore, the number of significant signals within an amino acid sequence is an indication of whether the amino acid sequence encodes a natural protein. For example, if the protein sequence being searched was generated from genomic DNA, the presence of more significant signals within a reading frame then expected by chance can indicate that a stretch of DNA encodes a protein.

Significant signals are also associated with particular structural features of proteins. Therefore, the presence and type of significant signals within an amino acid sequence can be used to predict structural features of a protein having the amino acid sequence. Significant signals show conservation between related proteins, for example, cognate proteins from different species. The identification of conserved significant signals between proteins can therefore be used to identify conserved structural features of the proteins, and therefore which segments of the proteins are critical for function. The conserved segments of proteins identified by conservation of significant signals are not coextensive with conserved segments identified by primary sequence analysis. Therefore, the described methods detect conserved regions of proteins that are missed by conventional approaches. For example, if the predetermined set of amino acids consists of C, I, L, M, F, W, Y and V, the following three amino acid sequences all generate the same signal (001011101) despite the fact that they contain no sequence identity: ANISVYYEM; TSFNFWMGV; SGCGLILNC. Three proteins that have these respective sequences at a particular position do not demonstrate amino acid similarity but their signal designations are identical. Significant signals that generate specific structural features of proteins can therefore predict common structural features between proteins the without need to rely on amino acid or nucleotide similarity to detect such features.

II. Useful Sets of Amino Acids

1. Generating Useful Sets of Amino Acids

Sets of amino acids useful in analyzing amino acid sequences are identified by testing one or more test sets of amino acids by the procedure described below. Any set of amino acids can be used as a test set. Because test sets are tested using computational methods, a very large number of test sets can be tested. For example, one can create every possible set of the twenty amino acids having from 2 to 19 members. Alternatively, one can test every possible set of the twenty natural amino acids having from 4-10 amino acids. One can also test sets of amino acids that can be defined using a priori classifications, such as basic (H, K, R), hydrophobic (A, C, I, L, M, F, P, W, Y and V), or the presence of a particular element in the side chain such as nitrogen (R, N, Q, H, K, P, W).

Test sets are tested on a collection of protein sequences. The collection of protein sequences can consist of any number of proteins. The proteins in the collection can be similar to each other, not similar to each other, or mixed in terms of similarity to each other.

Each protein in the collection is transformed into binary code by assigning amino acids within the proteins a first symbol if the amino acid is within the test set and a second symbol if it is not within the test set. For example, the amino acids in the test set can be designated as a 1 and all amino acids outside the test set can be designated as a 0. Of course, any other symbol can be used to designate an amino acid that falls inside or outside of a particular test set. The order of the symbols in a transformed amino acid sequence in each protein of the collection corresponds to the order of the amino acids in the protein sequence. Thus, the collection of protein sequences is transformed into a collection of sequences of symbols such as 1 and 0.

The sequences of symbols representing amino acids generated above are analyzed through a sequence window. The number of symbols within a window is referred to as the window length, designated as N_{w}. Windows are usually 5-15 symbols in length. A preferred length is 9 symbols. The number of amino acids represented by the window is the same as the number of symbols.

The signals present within the collection of sequences of symbols are determined. A signal is a pattern of symbols depicting the amino acids of a given set in a given sequence window. For a given window length and an amino acid sequence of known size, the number of signals in the amino acid sequence can be calculated. There are L-(P-1) signals per amino acid sequence, where L is the length of the amino acid sequence and P is the number of symbols in a window.

The signals can be classified by a criterion referred to as signal strength, Nₛₛ. The strength of a given signal is the number of amino acids for that window length that fall within the test set. For a window length of 9 symbols, the strength of any given signal is 0-9. A signal with a strength of 0 has no amino acids that fall within the test set. A signal with a strength of 6 has 6 amino acids that fall within the test set. For example, using a binary code in which any amino acid that is within the test set is designated as a 1, the signal 011001110 has an Nₛₛ of 5 while the signal 001010000 has an Nₛₛ of 2.

The collection of protein sequences, transformed into sequences of symbols using a test set of amino acids, can be represented as a distribution of signal strengths, referred to as signal strength distribution. For example, the signals 001011010 and 000011011 are different signals but both have a signal strength of 4. By calculating the occurrence of all signals of each signal strength, an observed signal strength distribution is constructed in which the number of occurrences of signals of each strength are calculated from the collection of protein sequences.

The expected signal strength distribution is also determined for the collection of protein sequences. The first step is to determine the frequency of each amino acid in the collection of protein sequences. The individual frequencies of the amino acids in the test set are added together to obtain the probability that any amino acid within the test set will occur in a given position in a protein. This value is referred to as fₐₐ. The expected numbers of all signals of each signal strength occurring are calculated based on fₐₐ. The expected numbers of all signals of each possible signal strength are then stratified to obtain an expected signal strength distribution. The expected signal strength distribution is then compared to the observed signal strength distribution.

If the probability that an observed signal strength distribution for a given test set occurs by chance is relatively low (e.g., preferably less than 1/10⁻¹⁰⁰, more preferably less than 1/10⁻⁵⁰⁰), the test set is useful for subsequent analysis. If the probability is not relatively low, the test set is not useful. Different sets of amino acids are identified that are useful, but many of these sets overlap in terms of the identity of their amino acids. Only a small proportion of all possible test sets are useful. However, very large numbers of test sets can be tested because the entire analysis can be performed using a computer.

A useful set can be improved through iterative cycles of analysis. A useful set generates a signal strength distribution that has a low probability of occurring by chance in the collection of protein sequences. To improve a useful set, one of the amino acids of the useful set is substituted for another amino acid not previously in the useful set to generate a modified useful set. Alternatively, a modified useful set can be generated by adding a new amino acid to or deleting an amino acid from the useful set. The signal strength distribution analysis is performed again on the modified useful set. The expected signal strength distribution for the modified useful set is modified compared to the previous analysis. The expected signal strength distribution is modified because the change to the identity of the amino acids in the useful set results in a different expected signal strength distribution. This is because fₐₐ differs for each unique test set of amino acids. In other words, by adding, deleting, or substituting an amino acid to the useful set the individual frequencies that are added to obtain fₐₐ changes. The amino acid change to the useful set can make the probability that the newly observed signal strength distribution occurs by chance lower or higher than the original probability for the useful set. If the amino acid change makes the probability for the new signal strength distribution lower, the modified useful set is more useful than the original useful set. If the amino acid change makes the probability for the new signal strength distribution higher, the modified useful set is less useful than the original useful set. Although preferred signal strength probabilities are in the range of 10⁻¹⁰⁰ to 10⁻⁵⁰⁰ or lower, the exact probability of a given modified test set is not critical. Rather, the important consideration is whether the modified test set generates a lower probability than the test set from which it was derived. Useful test sets generate low probabilities, however the most useful test sets generate local minimum probabilities that contain subsets of other useful sets. For example, the class 2 amino acids C, I, M, F, W, Y and V generate a local minimum, however many subsets of this set (such as C, I, M, F, W and Y or M, F, W, Y and V) are also identified as useful but are simply subsets of the most useful set C, I, M, F, W, Y and V.

The iterative process described above can lead to the identification of a set of amino acids that gives rise to a distribution of signal strengths that has a probability minimum. A set having a minimum probability distribution means a set for which the probability of chance occurrence of the distribution of signal strengths is less than the probability of chance occurrence of the distribution of signal strengths of any modified set representing an addition, substitution or deletion of an amino acid. Sets having a minimum probability distribution are preferred in subsequent methods of analysis.

2. Examples of Predetermined Sets

Two useful sets of amino acids have been identified using the analysis described above and in more detail in the Examples. One set is A, R, Q, E, L, K and M, also referred to as the class 1 set. The other set is C, I, L, M, F, W, Y, and V, also referred to as the class 2 set. Any single substitution, deletion, or addition to either set results in a higher probability that an observed signal strength distribution occurs in a collection of protein sequences by chance. Both sets were identified using a collection of 790 protein sequences containing 156,643 total residues with a window length of 9 residues. These sets are described further in Table 1. The collection of 790 protein sequences is listed in Table 3. Each protein in the collection of 790 proteins has 25% or less amino acid sequence identity with all other proteins in the collection.

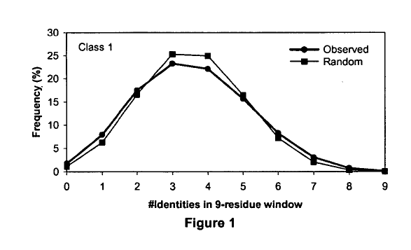

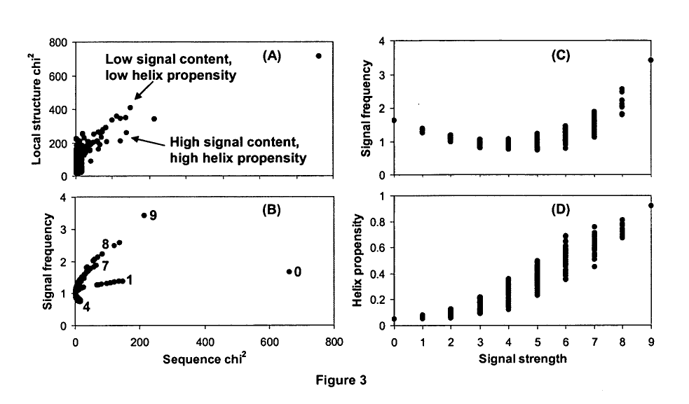

The class 1 set contains seven amino acids that vary in terms of charge, size, and hydrophobicity. When the class 1 set is used to transform the collection of 790 protein sequences, less signals occur having medium signal strength (3-5) than are expected by chance. This observed result, depicted in FIG. 1, is a significant deviation from the expected random signal strength distribution. The smaller frequency of signals of signal strength in the range of 3-5 than in the ranges of 0-2 and 6-9 suggests that either a low or high number of class 1 residues within a 9 amino acid window is a useful structural component of proteins. This observed signal strength distribution is depicted in FIG. 3(c). These results are discussed in more detail in Example 1.

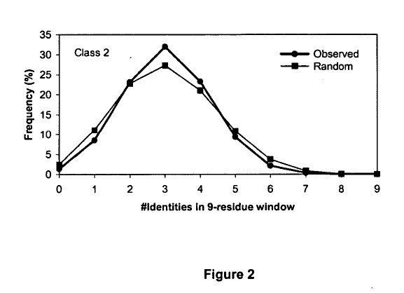

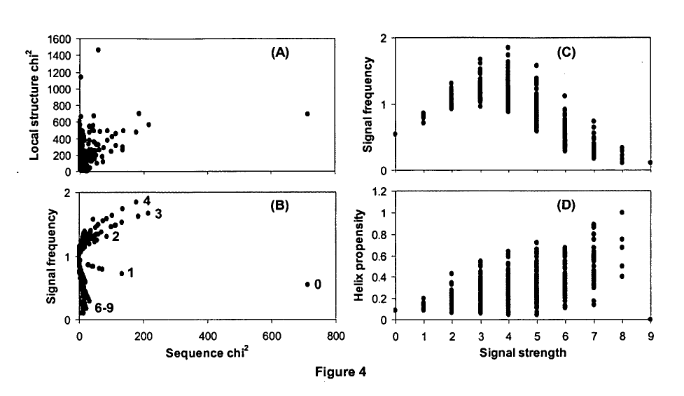

The class 2 set contains the eight most hydrophobic amino acids of the twenty naturally occurring amino acids. When the class 2 set is used to transform the collection of 790 protein sequences, more signals containing a medium signal strength (2-4) occur than are expected by chance. This observed result, depicted in FIG. 2, is a significant deviation from the expected random signal strength distribution. This signal strength distribution has a probability of occurring randomly in the collection of 790 proteins used in the analysis of 10⁻¹¹¹⁴; The maxima of signal strength distribution in the range of 2-4, depicted in FIG. 4(c), suggests that a medium number of class 2 residues within a 9 amino acid window is a useful structural component of proteins. These results are discussed in more detail in Example 1.

In the methods that follow, a predetermined set of amino acids is preferably defined as all of the amino acids in the class 1 set or all of the amino acids in the class 2 set, and no other amino acids. However, other predetermined sets of amino acids can be used based on the class 1 or class 2 sets. For example, one can define a predetermined set of amino acids to include 4-10 amino acids including at least 4 from the class 1 set. Alternatively, one can define a predetermined set of amino acids to include 4-10 amino acids including at least 4 from the class 2 set.

III. Analyzing Proteins Using Predetermined Sets

Query protein sequences can be analyzed using the class 1 and class 2 sets, or using other predetermined sets. Analysis is performed by transforming a query sequence into symbols according to a predetermined set of amino acids and analyzing the sequence for the presence of specific signals. The query sequence is transformed into a sequence of signals according to the symbols. The identities of up to L-(P-1) signals within the query amino acid sequence are determined, where L is length of the amino acid sequence and P is the number of amino acids in the window.

All possible signals for a particular window length and class of amino acids can given a designation. The designation for each signal can be arbitrary. For example, a signal can be given a designation of a number, such as 001 for the binary signal 000000000, 002 for 000000001, 003 for 000000010, 004 for 000000100, and so on, up to a designation of 512 for 111111111. The actual signals identified in the query sequence are identified as a sequence of these designations, corresponding to each signal present in each successive window. Usually the sequence of designations is generated in order from the N-terminus to the C-terminus. The sequence of designations corresponds to the signals in each successive window, which in turn correspond to the symbols generated by transforming the starting amino acid sequence. For a window length of 9, the first designation in the sequence corresponds to the specific signal created by the symbols that correspond to amino acids 1-9 of the amino acid sequence. The second designation in the sequence corresponds to the specific signal created by the symbols that correspond to amino acids 2-10 of the amino acid sequence, and so on.

As an example, the amino acid sequence PAGEQEAFPPN has 3 window lengths of 9. Transformed into a binary code according to the class 1 amino acid set, the sequence reads 00000000100. Using the designations mentioned in the above paragraph, the sequence of designations for this amino acid sequence using a window length of 9 reads 002-003-004. Any protein sequence can be transformed into a sequence of designations for any set of amino acids.

Higher order codes can also be created through the use of more than one predetermined set of amino acids. For example, an amino acid is assigned a first symbol if it is a member of a predetermined set of amino acids, and a second symbol different from the first symbol if the amino acid is not a member of the predetermined set. The second symbol can be a first type of second symbol if the amino acid is part of a second predetermined set of amino acids, and a second type of second symbol is assigned if the amino acid is not part of the second predetermined set of amino acids.

Higher order codes allow for the assignment of a symbol to any amino acid even if two or more predetermined sets of amino acids are used. For example, if the first set is A, R, Q, E, L, K and M and the second set is C, I, L, M, F, W, Y, and V, any amino acid falls into one of four possible groups. A first type of first symbol is assigned to amino acids that only fall into the first set (such as A). A first type of second symbol is assigned to amino acids that only fall into the second set (such as W). A second type of first symbol is assigned to amino acids that fall both sets (such as L or M). A second type of second symbol is given for amino acids that do not fall into either set (such as G).

The more sets that are used to transform a protein sequence into symbols, the more possible signals are created. For example, for a window of 9 and a binary code, there are 29, or 512 possible signals. For a window of 9 and a code of 2 symbols where one of the symbols has a first and second type, there are 39, or 19,683 possible signals. Designations can be given for each possible signal, such as designations of 001 through 512 in the first instance and 00001 through 19683 in the second instance.

There are a number of factors taken into account for determining an appropriate window length for analysis. The number of computations necessary to transform a query sequence into symbols and analyze it expands with each added symbol to the window length. The use of higher order codes also increases the necessary computations. The computational power of the computer system to be used is thus a consideration when performing the analysis.

IV. Assigning Grades to Signals

Signals identified in a collection of protein sequences can be assigned grades. A signal grade represents the probability that the observed frequency of a signal in a collection of protein sequences occurs by chance. One method of assigning grades is to assign one grade to a signal if it occurs significantly more or less frequently than by chance and a different grade if it occurs at a frequency expected by chance. Alternatively, grades can be assigned to signals by the degree to which their observed frequency differs from expected frequencies. In such a grading scheme, the lower the probability of an observed signal occurring by chance, the higher the grade assigned to the signal.

The probability of a given signal occurring by chance in a collection of sequences is calculated. Determining this probability requires first determining the frequency of each amino acid in a collection of sequences. The frequencies of each amino acid in the collection of sequences that are part of a predetermined set are added together to obtain the probability that an amino acid within the set will occur in any given position in a protein. This value is referred to as fₐₐ. Once fₐₐ is determined, the probability of a given signal of a given window length occurring at random is calculated. Examples of these calculations are found in Example 1. The expected number of occurrences of a signal is compared with the observed number of occurrences of the signal in the collection of sequences.

Signals can be classified as significant or not significant depending on whether the grade assigned to a signal exceeds a certain threshold. For example, a useful threshold is whether the observed frequency of a signal compared to the expected frequency of that signal produces a χ² value greater than 8. Alternatively, the threshold can be a value such as a χ2 value of greater than 4, 10, 20, 50, or higher.

For example, consider the signal 001100100, for the predetermined set of class 2 amino acids C, I, L, M, F, W, Y and V. This signal occurs 801 times in the collection of 790 proteins in Table 3 but is expected to occur only 479 times in random sequences of equal length and amino acid composition. The signal frequency is therefore 801/479, or 1.67. Signal frequency may be sub or super unity, and statistically significant signals may have low or high frequencies. For this reason, significance is determined through χ² values. For the 801/479 observed/expected ratio for the signal 001100100 the χ² value is 216.3, indicating that this signal is significant. If instead this signal only occurred 522 times, the χ² value would be 3.9 (below the threshold of 8), indicating that this signal would not be significant.

A protein sequence may be transformed into a sequence of grade designations corresponding to each successive signal in the sequence. For example, signals with significant grades can be referred to by designations that identify the signal, while signals that are not significant can be given a common designation of a 0. The sequence of designations therefore conveys two pieces of information for each designation in the sequence. The first piece of information is whether or not the signal at that position is significant. If the designation is a 0, the signal is not significant. If the designation is any number other than a 0, the signal at that position is significant. The second piece of information, for those signals that are significant, is which signal occurs at that position. An example of such a sequence is 0-0-213-0-327-0, where the 0s designate non significant signals and 213 and 327 designate significant signals of a particular identity. For example, see the sequences of signal designations for three globin proteins in Table 2. Alternatively, designations can convey only whether or not the signal at that position is significant. In this instance only two designations are used.

Significant signals are identified from a collection of proteins and used in subsequent analysis. For example, the number of significant signals in a query amino acid sequence can be determined. Also, the identity of specific signals and their location in the sequence of a query protein can be compared to the identity and location of signals in other proteins. Examples of these applications are discussed in later sections.

V. Amino Acid Sequences that can be analyzed

Any amino acid sequence, regardless of source, can be analyzed using the methods. For example, naturally occurring protein sequences can be analyzed. Such naturally occurring proteins are obtained from databases or from experimental data.

Theoretical proteins encoded by different reading frames of DNA can also be analyzed using the methods. Theoretical protein sequences arise from sequencing genomic DNA, cDNAs, or from translating nucleotide sequences from databases.

Hypothetical amino acid sequences can be analyzed for the presence of significant signals. For example, proteins proposed for synthesis can be can be analyzed for the presence of significant signals. These hypothetical proteins can arise from any source, such as computer programs or manual design.

VI. Representative Applications

1. Identifying Coding Regions in DNA

Genomic DNA sequences can be analyzed to determine if they encode proteins. To identify all possible protein sequences encoded by a DNA sequence, the DNA sequence is translated into each of three reading frames in both directions. As such, a given segment of DNA could encode 6 different theoretical protein sequences. Each of the 6 translations of the DNA sequence are scanned for the presence of significant signals using a predetermined set of amino acids such as classes 1 and 2. A reading frame that contains more significant signals than would be expected by chance is more likely to encode a protein than the other reading frames that do not encode such a high frequency of significant signals. Whereas random protein sequences have very few specific signals with χ² values greater than 8, actual protein sequences contain many such signals.

Once significant signals have been identified, their expected frequency in coding sequences can be calculated. The expected distribution of significant signals is then compared to the observed distribution in a stretch of DNA. The χ² value is then computed, and from this value the probability that the nucleotide sequence is a coding sequence can be calculated. The lower the probability that the significant signals occur by chance in a given stretch of nucleic acid sequence, the higher the chance that the sequence encodes a protein. This method can be applied to any nucleotide sequence, regardless of the origin of the sequence.

In a conventional analysis all 6 translations would be compared against databases of known proteins. If one of the reading frames encodes a protein with significant sequence similarity to other known proteins, the correct reading frame can be identified. If no protein sequence is identified however, it is not possible using this conventional method to determine if the DNA segment encodes an unknown protein. The present methods can be used to determine if such a segment encodes a protein regardless of protein or DNA sequence similarity to other known proteins.

Signal analysis can identify reading frames in expressed sequence tag (EST) sequences. When EST sequences are isolated they frequently do not encode the full length of a protein. In addition, any start or stop codons in the sequence have only a 1 in 6 chance of being in the correct reading frame, making it difficult to determine which start or stop codons in the sequence, if any, are the actual start or stop codons of the protein encoded by the EST. As with genomic sequence analysis, conventional EST sequence similarity searching of all 6 translations only works when the encoded protein has sequence similarity to known proteins. Signal analysis can be used to identify the correct reading frame by identifying significant signals in each reading frame of the EST sequence.

As in the instance of genomic DNA analysis, the lower the probability that the significant signals occur by chance in a given reading frame of an EST, the higher the chance that the reading frame is the actual reading frame for translation of that sequence.

2. Comparing Protein Sequences

The signals in two or more protein sequences can be compared. Each sequence is transformed into symbols using a predetermined set of amino acids. Signals are determined after designating a particular window length. The signal patterns of the proteins are then compared. Optionally, each signal pattern is converted into a sequence of signal designations with significant signals identified as a particular signal identity and nonsignificant signals designated with a 0. Both sequences of signal designations are analyzed for the conservation of significant signals. When using useful sets of amino acids such as classes 1 and 2, this analysis reveals structural conservation in the absence of amino acid sequence similarity or identity. Additionally, once the sequences of signals designations are generated, the sequences can be aligned for maximum conservation of significant signals. For an example of such a comparison, see Table 2.

3. Predicting Local Structure

Significant signals are correlated with the presence of certain secondary structures. A library of canonical structural fragments that represent recognized secondary structure motifs can be analyzed for an above-expected occurrence of a particular signal that is correlated with a particular structural motif. Particular secondary structure motifs correspond to particular signals. For example, the frequency with which each fragment from a library of 17 structural motifs is associated with certain signals of the class 2 amino acids can be calculated. The occurrence of these signals in a collection of 790 proteins is analyzed by calculating which structural motifs correspond to each occurrence of these signals. The collection of 790 protein sequences was obtained from the Protein Data Bank, which stores the three dimensional structures of proteins. The structure of the amino acid sequence corresponding to each occurrence of class 2 signals 28, 290, 66, and 358 in the collection of 790 proteins was analyzed. These signals occur at a much higher frequency than expected by chance in certain structural motifs. These correlations are shown in Tables 16 and 17. Signals 28, 290, 66, and 358 are correlated with the centroids alpha helix, beta hairpin, extended helix, and beta strand, respectively. These methods are further discussed in Example 1.

Signals are identified from a collection of protein sequences to be correlated with a particular structural motif, or “local structure centroid.” The occurrence of these signals in a query protein is used to predict secondary structure. Examples of such secondary structure are alpha helix, beta strand, beta turn, turn+beta, helix+turn, helix cap, extended helix, Gly/Pro twist, beta+turn, helix-hairpin, beta cap, helix hairpin, beta hairpin, contorted helix, turn, helix+turn II and helix turn. Each local structural motif, or centroid, has a natural abundance. For the a set of proteins, such as the set of 790 proteins in Table 3 with non redundant sequences, the frequency of each centroid can be measured. Each centroid has a corresponding amino acid sequence, and therefore a corresponding signal, for a given predetermined set. A list of centroids and their associated signals can be generated. All centroids associated with a given signal are compiled, generating a calculation of the abundance of each centroid in the presence of the given signal. These abundances are compared with the natural abundances computed above (ie, from all 790 proteins without consideration of sequence). For signals with high sequence χ² values, the associated centroids have significantly different abundances than the natural abundances. Some centroids are more frequent than generally expected while others are less frequent. These associated signals can be used to predict secondary structure. These calculations narrow the probabilities of a centroid being associated with a particular signal compared to the natural abundances of the centroids. These methods are therefore used to predict secondary structure, and are superior to traditional secondary structure prediction in two ways. First the methods predict structure using a much finer categorization of structure (e.g., choosing from the above-described centroids instead of the conventional categories of helix, strand, coil, or unknown). The local structure centroids are actually defined by X,Y,Z (Cartesian) coordinates of the alpha carbon (backbone) atoms, rather than merely a qualitative description of conventional methods. Second, these methods produce a vector of probabilities that sums to unity. In every case, given the presence of a signal, the methods generate the probability of all centroids. Conventional secondary structure prediction simply gives a prediction with no probability In fact, at many loci conventional methods return a null value.

4. Predicting Protein Fold

Signal analysis can be used to predict the fold of a protein with a particular amino acid sequence. The fold of a protein encoded by a query sequence can be determined by comparing the position and identity of signals in the query sequence to conserved signal patterns in families of proteins. Between proteins from the same family, some specific signals occur in similar positions at a higher frequency than would be expected by chance. “Similar positions” means that the specific signals that recur in protein families occur in approximately the same region of proteins in the family relative to the N and C terminii of the proteins, as demonstrated in following sections and in Example 2. The conservation of such specific signals is indicative of the specific signals in a particular region of the protein playing a role in generating and maintaining a specific fold. Although proteins of a certain family usually possess amino acid sequence similarity, such similarity is only partially indicative of retained structure. As mentioned previously, two amino acid sequences can generate the same signal in a given window despite having no amino acid identity. The methods therefore detect structural signals that may be missed in conventional amino acid sequence comparisons. Signal pattern conservation identified by these methods is a more fundamental type of conservation between proteins than amino acid sequence conservation.

To identify specific signals and relative positions of specific signals that are conserved in protein families, a plurality of protein sequences of a given family are analyzed. Each member of a family of related proteins used in the analysis can be first individually compared to each other member in the family. Preferably, no member of the family has more than 90% amino acid sequence identity to another member of the family in the collection of proteins to be used. Preferably, all members of the family used in the analysis possess at least 20% amino acid identity with each other. Optionally, a plurality of families of proteins are collected and analyzed. Each member of each family is then separately analyzed for amino acid identity in the manner described above.

For example, a plurality of members of a the following protein families were collected: globins, lysozymes, thioredoxins, trypsins, monoclonal antibodies, and amido transferases. Each sequence collected was compared to each other sequence from the same family. If a comparison between two members of a family produced more than 90% amino acid identity between the two, one of the sequences was removed from the collection. In addition, no sequences were kept in the collection if they did not possess at least 20% amino acid sequence identity with all other proteins in the collection for that family. Tables 7-13 contain accession numbers for each member of each family of proteins used in the fold analysis that met the ≧20%-≦90% criteria.

Each collection of proteins in a family is transformed into sequences of signal designations using methods described in earlier sections. For example, each family is separately transformed into signals according to amino acids of classes 1 (A, R, Q, E, L, K and M) and 2 (C, I, M, F, W, Y and V). For each class of amino acids, all members of a family are analyzed and compared to each other for the presence of specific signals that occur in similar positions. Some signals are identified as conserved between members of a family at a similar position in each protein.

The probability that a signal appears at a certain point relative to the beginning-and end of a protein in a protein of that family is calculated using a location probability density function (PDF). The precise method used to calculate the PDF can be based on the fast Fourier transform (FFT). This method computes Gaussian kernel estimates of a univariate density using the FFT over a fixed kernel interval. For examples of these calculations, see Example 2. Preferably, a kernel width value of 0.05 relative to the overall length of the protein is used to calculate PDFs. The PDFs for each are family of proteins are calculated for each class of amino acids. The PDF data for each family is then used to analyze a query sequence to determine if the query sequence contains a similar conserved pattern of specific signals in similar locations.

Optionally, conventional sequence alignment techniques can be used to demonstrate conserved signal patterns, such as inserting gaps into strings of signal designations and/or sliding them relative to each other to achieve maximum signal pattern conservation. In some methods, signals are allowed to skip over a gap, such that a 9 residue signal can occur over a 9 residue stretch of sequence that contains a few gaps. In other methods, signals are not allowed to extend over a gap. Conventional sequence comparison methods such as BLAST can be used for this purpose.

A query amino acid sequence is transformed into symbols as described in previous sections. The query sequence is transformed into a sequence of signal designations according to the same amino acid classes as used to transform the families of proteins. The transformed query sequence is then analyzed for the presence of the same specific signals at similar locations as those that are conserved in families of proteins. The likelihood that a given sequence of signals codes for a given fold can be determined, for example, using Bayes' rule as demonstrated in Example 2.

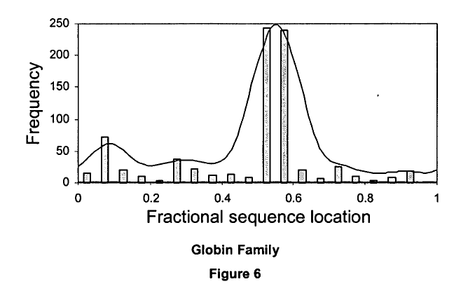

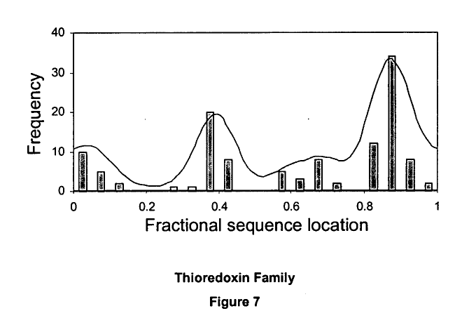

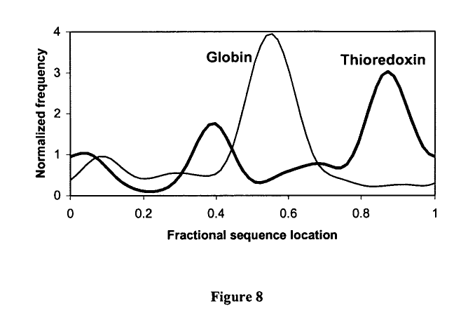

In general, the signal occurrences across a sequence are correlated. That is, for a given protein family, all members of the family of proteins have the same signal in a similar position. Alternatively, all members of the family of proteins have the same first signal at a first position and at least one additional signal at a second position. In this instance the identities of the first and additional signals can be but do not need to be the same. The presence of both signals at each respective location in the protein is necessary to generate a specific fold. The identity and relative location of signals in a query amino acid sequence are therefore compared to conserved patterns of specific signals of all members of a family of proteins that have a common fold. For example, the signal designated as signal 92 (000100100), derived from the class 2 amino acids, occurs at a moderate frequency in the thioredoxin family at approximately 40% of the way thorough the sequence and at a higher frequency at approximately 90% of the way thorough the proteins. (see FIGS. 7 and 8). By contrast, the same signal (92) derived from the class 2 amino acids occurs at a high frequency in the globin family at approximately 50-60% of the way through the sequences of members of this family. (see FIGS. 6 and 8). These recurring signals at similar locations in the families of proteins are conserved signal patterns. Conserved signal patterns can consist of more than one specific signal.

The position and identity of signals in a query amino acid sequence are compared to the position and identity of specific signals that are conserved between members of the same protein family. The result of the comparison is referred to as fold score. The closer the query pattern of signals is to a conserved signal pattern found in a particular family, the higher the fold score the query is given for that family. When the query is compared to different families of proteins, each comparison generates a fold score. The family that generates the highest fold score for a query sequence is more likely to possess a similar or identical fold to the query sequence than families that generate a lower fold score for that query sequence. Additional examples of fold score calculations are in Example 2.

Two or more signals at different positions in a protein, each or all of which are required to generate the fold of the protein, are higher-order signals. As opposed to the signals described in earlier sections which comprise a single stretch of contiguous symbols, higher order signals comprise two or more signals that recur in a family of proteins in distinct regions of the proteins. A higher order signal therefore comprises at least two signals that are non-overlapping, each of which are required for a protein to adopt a particular fold. Protein families with more than one conserved non-overlapping signal possess higher-order signals.

5. Predicting the Structure of Hypothetical Proteins

Proteins can be designed for a specific function based on knowledge of preexisting protein structures and functions. To change the function of a known protein, some amino acid sequence change must usually be made. Hypothetical sequences designed to carry out a novel function can be scanned for the presence of signals. The signals identified in the hypothetical sequence are compared against signals from a preexisting protein that the hypothetical protein is based on. Preferably, the signals identified in the hypothetical sequence are compared against signals from a family of preexisting proteins with a known fold. The comparison reveals whether significant signals or conserved signal patterns present in the preexisting protein or protein family, respectively, are destroyed by the proposed sequence changes. The comparison also reveals whether new significant signals are created by the amino acid sequence changes. Sequence changes that destroy significant signals or conserved signal patterns are more likely to alter the fold of a protein than sequence changes that change non-significant signals or signals that are not conserved between members of a family.

6. Predicting Structure of Variant Proteins

In a similar fashion as in the preceding section, naturally occurring variants of proteins can be analyzed for conservation of signal patterns. For example, signal patterns for a family of human proteins can be established based on known sequences. A gene encoding a protein from this family is then amplified from DNA or RNA in tissue samples and sequenced. The amplified gene sequence is translated in the correct reading frame and transformed into signals.

Single nucleotide polymorphisms (SNPs) or other mutations can be present in the amplified genes that alter the amino acid sequences of the proteins. Variant nucleotide sequences are translated into amino acid sequences and analyzed for conservation of significant signals or conserved signal patterns. SNPs or other mutations that alter significant signals or conserved signal patterns are more likely to cause structural perturbations of the protein than SNPs or other mutations that do not alter significant signals or conserved signal patterns. In addition, the variant sequences can be analyzed for the gain of a significant signal due to a sequence mutation or polymorphism.

VII. Computer implementation

1. Suitable Computer Systems

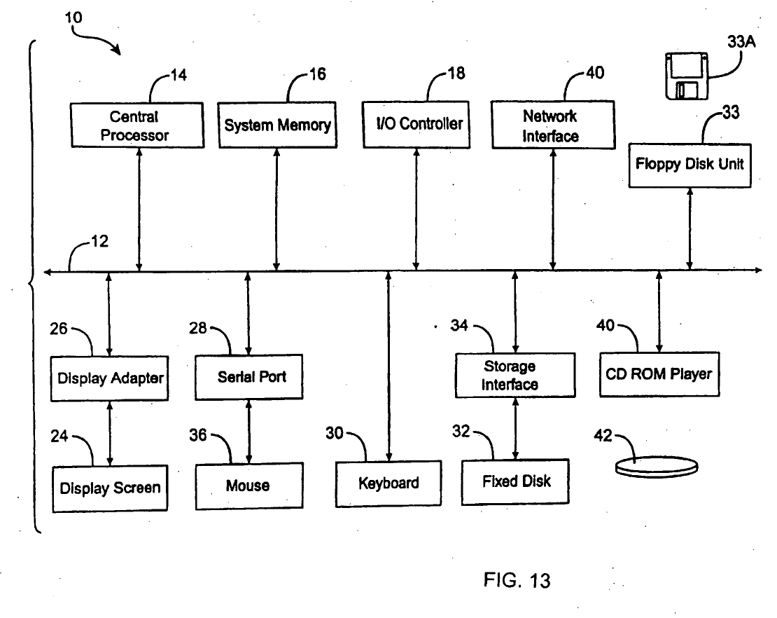

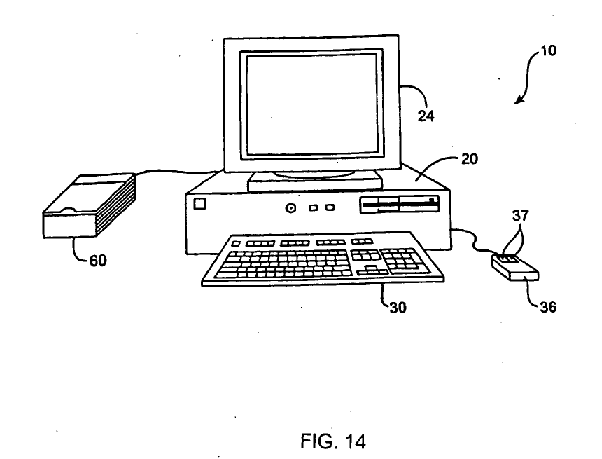

A computer system is preferably used for implementing the methods, as depicted in FIGS. 13 and 14. As depicted, a suitable computer system 10 includes a bus 12 which interconnects major subsystems such as a central processor 14, a system memory 16, an input/output controller 18, other storage means, an external device such as a printer 20 via a parallel port 22, a display screen 24 via a display adapter, a serial port 28, a keyboard 30, a fixed disk drive 32 and a floppy disk drive 33 operative to receive a floppy disk 33A. Such components can be contained in a cabinet. Many other devices can be connected such as a scanner (not shown) via I/O controller 18 and a mouse 36 connected to serial port 28 or a network interface 40. A mouse and keyboard are “user input devices.” Other examples of user input devices are a touch screen, light pen, track ball, data glove, etc.

Many other devices or subsystems may be connected in a similar manner. Also, it is not necessary for all of the devices recited be present to practice the present invention, as discussed below. The devices and subsystems may be interconnected in different ways. The operation of a computer system such as that described is readily known in the art and is not discussed in detail in the present application. Source code to implement the present invention may be operably disposed in system memory or stored on storage media such as a fixed disk or a floppy disk.

In a preferred embodiment, System 10 includes a Pentium® class based computer, running Windows® Version 3.1, Windows95®, Windows98®, WindowsXP®, or WindowsME® operating system by Microsoft Corporation. However, the method is easily adapted to other operating systems without departing from the scope of the present invention.

The mouse 36 may have one or more buttons 37. As used in this specification, “storage” includes any storage device used in connection with a computer system such as disk drives, magnetic tape, solid state memory, and bubble memory. The cabinet 20 may include additional hardware such as an input/output (I/O) interface 18 for the connecting computer system 10 to external devices such as a scanner, external storage, other computers or additional peripherals.

2. Flowcharts Depicting Examples of the Methods.

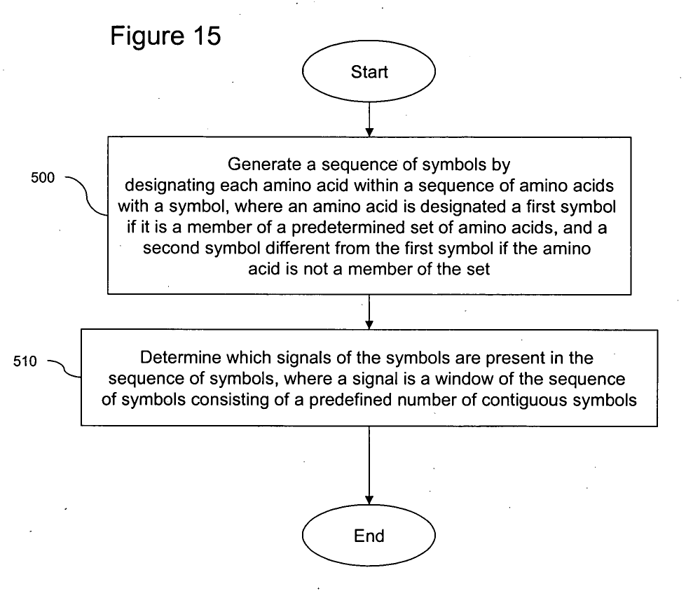

Suitable computer systems can perform the described methods using software that performs functions as depicted in the flowcharts in FIGS. 15-21. The steps of the flowcharts can be executed by software or hardware or combinations thereof. In addition, the steps can be executed by a single computer or multiple computers acting in combination. The information received by the computer can be input by the user or received from any other source, including a memory location that can be accessed by a machine running software that performs the methods.

FIG. 15 depicts a flowchart of simplified steps for a computer-implemented method of analyzing a sequence of amino acids. In a step 500, a sequence of symbols is generated by designating each amino acid within a sequence of amino acids with a symbol, where an amino acid is designated a first symbol if it is a member of a predetermined set of amino acids, and a second symbol different from the first symbol if the amino acid is not a member of the set. In a step 510, which signals of the symbols are present in the sequence of symbols is determined, where a signal is a window of the sequence of symbols consisting of a predefined number of contiguous symbols.

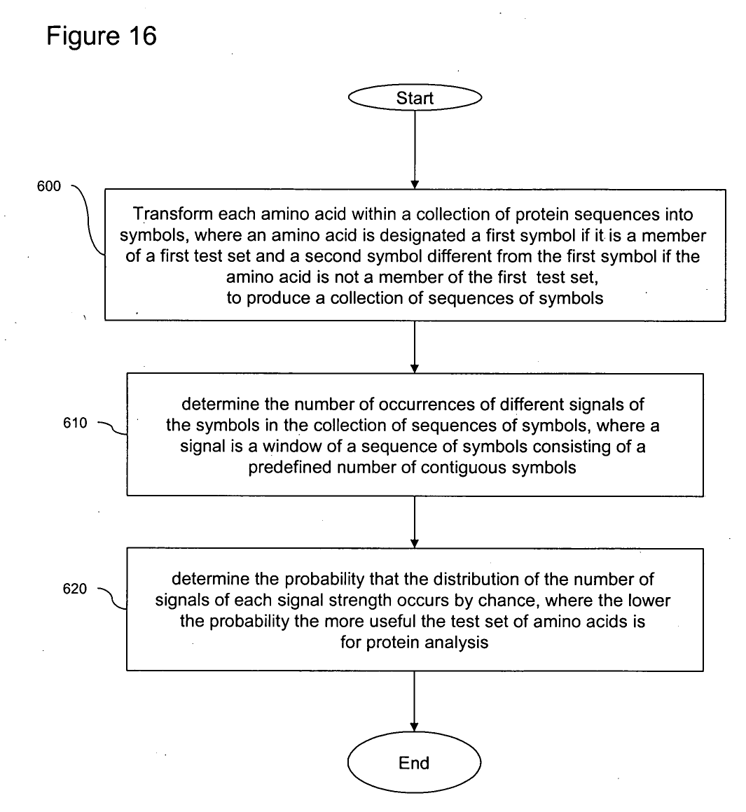

FIG. 16 depicts a flowchart of simplified steps in a representative embodiment for identifying a set of amino acids useful for the analysis of proteins. In a step 600, each amino acid within a collection of proteins sequences is transformed into symbols, where an amino acid is designated a first symbol if it is a member of a first test set and a second symbol different from the first symbol if the amino acid is not a member of the first test set to produce a collection of sequences of symbols. In a step 610, the number of occurrences of different signals of the symbols in the collection of sequences of symbols is determined, where a signal is a window of a sequence of symbols consisting of a predefined number of contiguous symbols. In a step 620, the probability that the distribution of the number of signals of each signal strength occurs by chance is determined, where the lower the probability the more useful the test set of amino acids is for protein analysis.

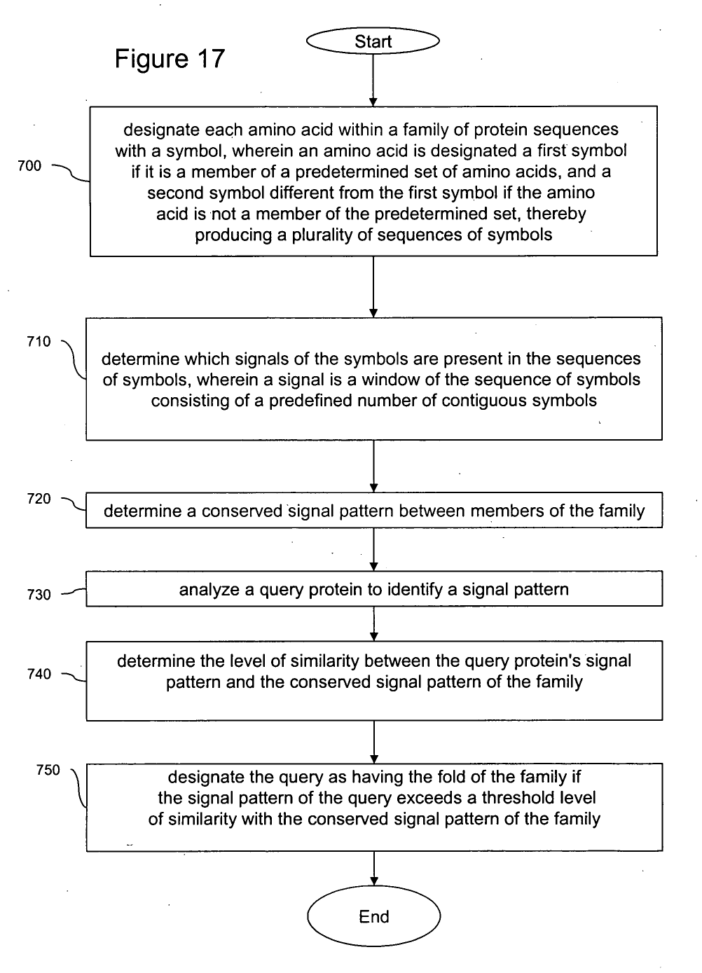

FIG. 17 depicts a flowchart of simplified steps in a representative embodiment for predicting the fold of a protein. In a step 700, each amino acid within a family of protein sequences is designated with a symbol, wherein an amino acid is designated a first symbol if it is a member of a predetermined set of amino acids, and a second symbol different from the first symbol if the amino acid is not a member of the set, thereby producing a plurality of sequences of symbols. In a step 710, which signals of the symbols are present in the sequences of symbols is determined, wherein a signal is a window of the sequence of symbols consisting of a predefined number of contiguous symbols. In a step 720 a conserved signal pattern between members of the family is determined. In a step 730 a query protein is analyzed to identify a signal pattern. In a step 740 the level of similarity between the query protein's signal pattern and the conserved signal pattern of the family is determined. In a step 750 the query is designated as having the fold of the family if the signal pattern of the query exceeds a threshold level of similarity with the conserved signal pattern of the family.

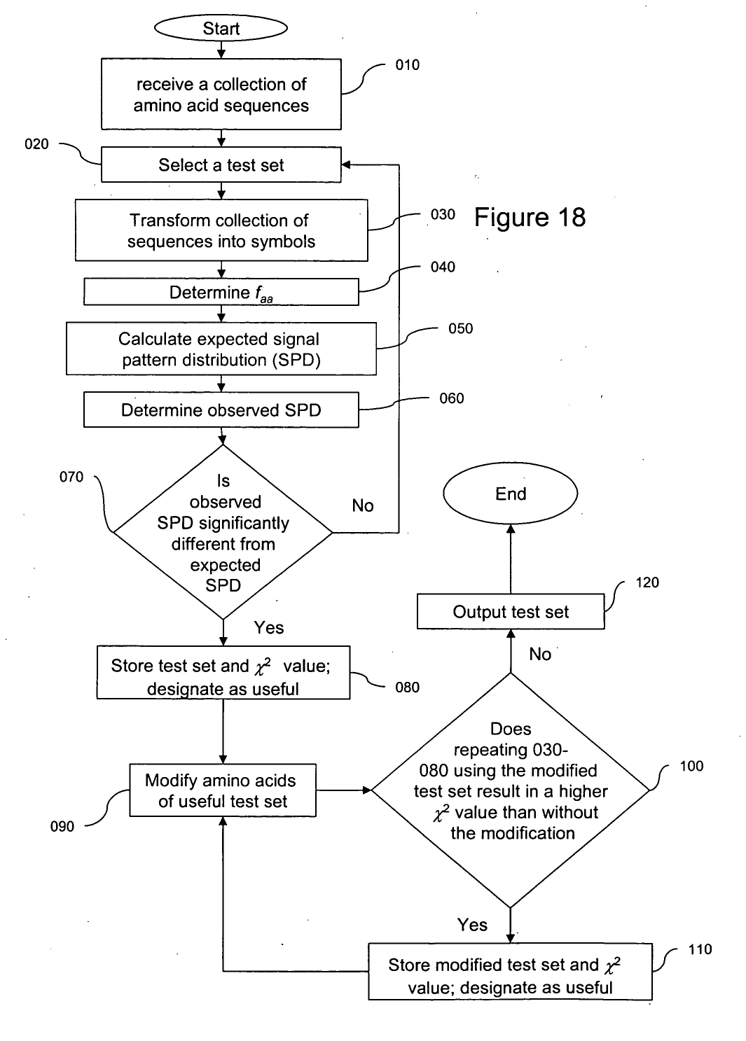

FIG. 18 depicts a more detailed flowchart of simplified steps in a representative embodiment for identifying a useful set of amino acids. In a step 010, a collection of amino acid sequences is received into a computer system. In a step 020, a test set is selected, and in a step 030 subsequent to both 010 and 020, the collection of amino acid sequences is transformed into sequences of symbols. In step 030, an amino acid is designated as one symbol if it falls within the test set and a different symbol if it is not in the test set. The frequency of occurrence, in the collection of sequences, of each amino acid in the test set is calculated in step 040. In a step 050 the expected signal strength distribution (SSD) is determined for the collection of amino acid sequences. The observed SSD is calculated in a step 060. In a decisional step 070 the observed and expected SSD measurements are compared. If the expected and observed measurements are not significantly different from each other, the test set is not useful. A new test set is then selected as steps 020-070 are repeated with a new test set. If the expected and observed measurements are significantly different from each other in decisional step 070, the test set is useful and is designated as such in a step 080. In a step 090, an amino acid is then deleted, added, or substituted in the test set, generating a modified test set. Steps 030-080 are then performed using the modified useful test set, and the results are analyzed in decisional step 100. If the modification makes the modified useful test set generate a SSD that differs from what is expected more than the useful test set on which it is based, the modified useful test set is stored and designated as useful in a step 110. In step 090 the modified useful test set is then subjected to an additional modification and steps 030-080 are repeated until it is determined in decisional step 100 that any further modification to the useful test set results in a lower level of statistical significance than without the modification. Once this determination is made, the useful test set is then output in a step 120.

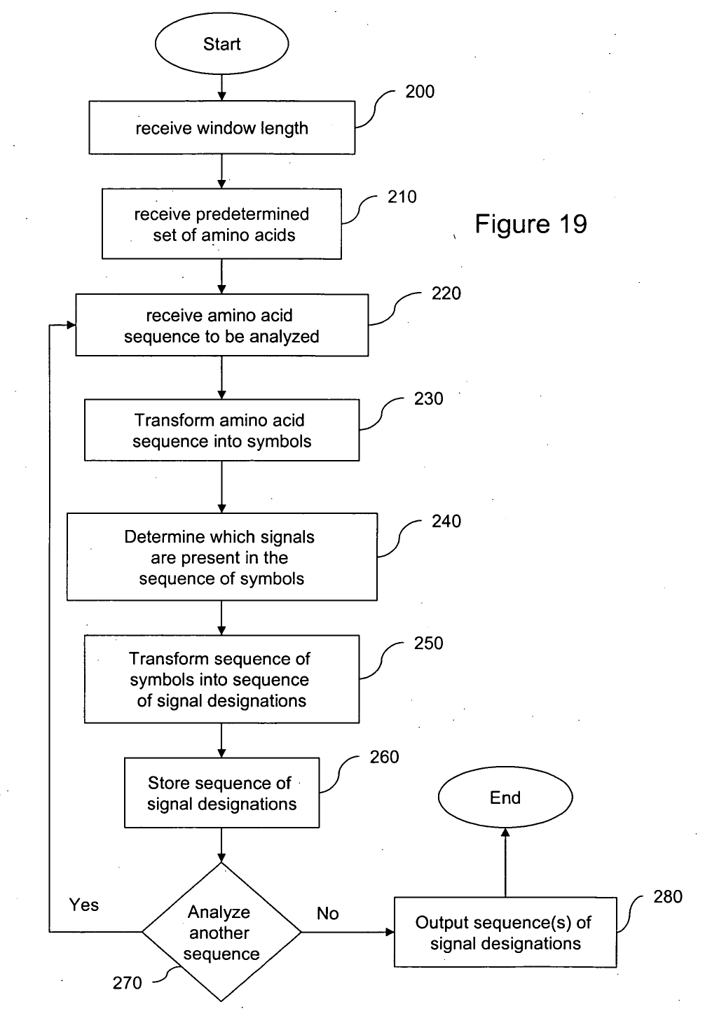

FIG. 19 depicts a more detailed flowchart of simplified steps in a representative embodiment for identifying signals in one or more amino acid sequences. In a step 200, the window length to be used in the analysis is received. In a step 210, a predetermined set of amino acids to be used in the analysis is received. Alternatively, the computer used in the analysis can already have such information as a default. In a step 220, an amino acid sequence to be analyzed is received by the computer. In a step 230, the amino acid sequence is transformed into a sequence of symbols using the designated predetermined set. In a step 240, the signals in the sequence of symbols are determined according to the designated window length. In a step 250, the sequence of symbols is transformed into a sequence of signal designations. In a step 260, the sequence of signal designations is stored. In a decisional step 270, it is determined if another amino acid sequence is to be analyzed. If the answer is yes, steps 220-260 are repeated using the additional amino acid sequence. If the answer is no, in a step 280 the sequence(s) of signal designations are output.

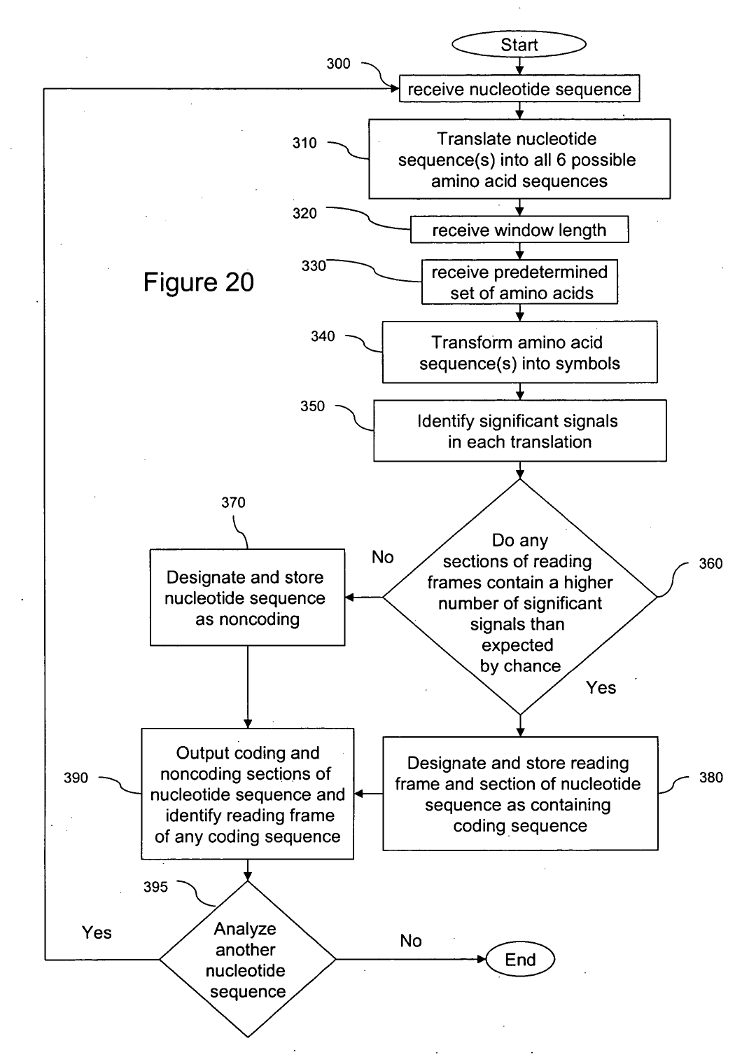

FIG. 20 depicts a flowchart of simplified steps in a representative embodiment for scanning a nucleotide sequence for the presence of reading frames that contain significant signals. In a step 300, a nucleotide sequence is received by the computer. In a step 310, the received nucleotide sequence is translated into 6 reading frames; 3 of the reading frames are according to each possible reading frame reading from 5′ to 3′ on one strand of the nucleotide sequence while the other 3 reading frames are according to each possible reading frame reading from 5′ to 3′ on one strand on the complementary strand of the nucleotide sequence. In a step 320, a window length to be used in the analysis is received. In a step 330, a predetermined set of amino acids to be used in the analysis is received. In a step 340, all 6 translations are transformed into sequences of symbols. In a step 350, significant signals are identified in each translation. In a decisional step 360, each translation is analyzed for the presence of more significant signals than expected by chance. The user can specify the minimum acceptable probability that is used to identify a coding region, or alternatively a default value can be used. If the number of significant signals in a translation exceeds the threshold, the translation is designated as corresponding to an actual coding sequence in a step 370. If the number of significant signals in a translation does not exceed the threshold, the translation is designated as corresponding to an non coding sequence in a step 360. In a step 390, the coding and noncoding sections of the nucleotide sequence are outputted as well as the reading frame of any coding sequence. In a decisional step 395, it is determined if any additional nucleotide sequences are to be analyzed. If the answer is yes, the additional nucleotide sequence is received and steps 310-395 are repeated. If the answer is no, the method is concluded.

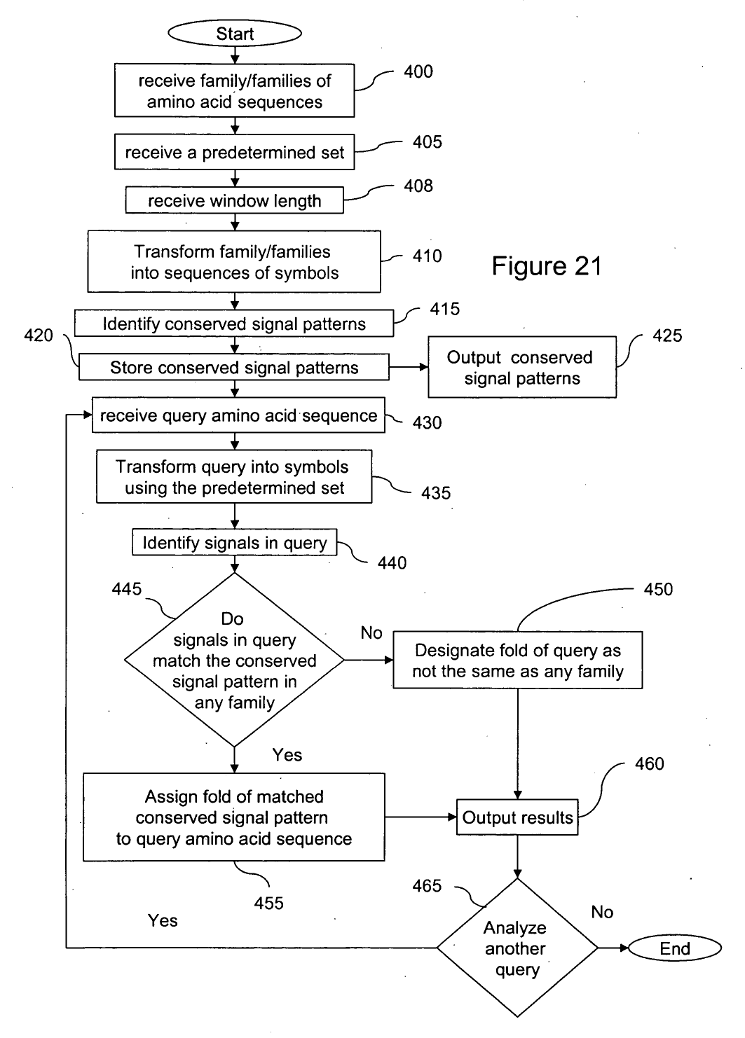

FIG. 21 depicts a more detailed flowchart of simplified steps in a representative embodiment for predicting the fold of a query protein. In a step 400, one or more families of amino acid sequences are received by the computer. In a step 405, a predetermined set of amino acids to be used in the analysis is received. In a step 408, a window length to be used in the analysis is received. In a step 410, all families of amino acid sequences are transformed into sequences of symbols. In a step 415, conserved signal patterns between members of a family are identified. In a step 420, the conserved signals pattern(s) are stored. In a step 430, a query amino acid sequence is received by the computer. In a step 435, the query amino acid sequence is transformed into sequence of symbols using the same window length and predetermined set as received in steps 405 and 408. In a step 440, signals are identified in the query amino acid sequence. In a decisional step 445, it is determined if any signals in the query sequence match a conserved signal pattern in any family transformed in step 410. If the answer is yes, the fold of the query sequence is designated as the same fold as that family, and the fold is assigned to the query in a step 455. If the answer is no, the fold of the query sequence is designated as not the same fold as any family in the analysis in a step 450. The results of steps 450 and 455 are output in a step 460. In a decisional step 465, it is determined if another query sequence is to be analyzed. If the answer is yes, another query amino acid sequence is received by the computer in step 430 and steps 435-465 are repeated. If the answer is no, the method is concluded.

Amino acid sequences to be analyzed by the aforementioned methods can be inputted by a user into a computer system. The sequences can also be downloaded from databases by a user or by the computer. For example, the sequences can be downloaded from public databases such as Swiss-Prot and NCBI. Alternatively they can be downloaded from internal databases or servers. The sequences can also be inputted into the computer system manually. Steps of selecting a predetermined set and a window length can be skipped if the computer system or software has these values selected as defaults.

As can be appreciated from the disclosure above, the present invention has a wide variety of applications. Accordingly, the following examples are offered by way of illustration, not by way of limitation.

### EXAMPLE 1

Just as written languages appear random unless one knows the words, so too protein sequences appear as random. By statistical measures they are far from random. Consider the number of times the amino acid alanine occurs in a protein sequence segment of nine residues. If the sequence were random, then the binomial distribution indicates that there is a 47% chance of zero alanines occurring, 37% of one alanine occurring, 13% chance of two alanines occurring, and so forth. We do not observe these frequencies in real protein sequences.

The probability that the observed alanine frequencies arose from a random parent population of protein sequences is about 10-310. The distribution of alanine residues in real protein sequences is not close to being random. Alanine is not unusual in this regard and other amino acids are even more non-random. This non-randomness is the result of patterns in the protein sequences which are repeated, just as letter patterns and words are repeated in written languages.

The same analysis can be performed on English text with similar results. For example, again using a nine-word text window, we find that the observed frequency distribution of the letter “A,” in a 70,000 word sample of English text, has a probability of about 10⁻¹¹³⁰ of arising from a random parent population. Interestingly, the vowels (A, E, I, O, U and Y), taken together as a group of like characters, form an optimal set. Their frequency distribution has a probability of about 10⁻³¹⁰⁰⁰ of arising from a random parent population, and this is a minimum point in letter space. We obtained a greater probability of arising from a random parent population if any of the vowels is removed from the set, or if any other letter is added to the set.

The eight most hydrophobic amino acids (cysteine, isoleucine, leucine, methionine, phenylalanine, tryptophan, tyrosine and valine), taken together as a group of like monomers has a frequency distribution with probability of about 10⁻¹¹¹⁴ of arising from a random parent population. This probability increases if any of these amino acids is removed from the set, or if any other amino acid is added to the set.

These results, based on statistical analysis, directly correspond to the chemistry of the amino acids. The hydrophobics form a nonrandom set of amino acids. No knowledge of chemistry was necessary to obtain these results, yet the results correspond to the chemistry of this set of amino acids.

In this Example, we show that protein sequences contain nonrandom signals. The signal can be associated with structure and function. We describe methods to search for and identify such signals, present our findings of two signal classes and the characteristics of their signals, and describe some representative applications of these signals.

1. Identifying Classes of Amino Acids

On the hypothesis that protein sequences contain non random signals we looked for patterns using a collection of 790 protein sequences that contain a total of 156,643 residues. To avoid weighting our results toward heavily studied protein families we restricted our collection of 790 protein sequences to non redundant sequences using a 25% sequence identity threshold (PDB codes of the 790 sequences are listed in Table 3).

We used a binary signal model in which each of the 20 amino acids was assigned a value of 0 or 1. We defined signals as the pattern of 1's that appears in protein sequences when transformed using the model. For example, consider the ARQELKM amino acid set. A protein sequence was transformed by assigning a 1 to all residues that are members of the set of those seven amino acids, and assigning a 0 to all other residues. If we used a sequence window nine residues in length, then there are a total of 2⁹, or 512 different possible signals. The signal strength for each signal, Nₛₛ, is the number of selected amino acids in the particular signal, or equivalently the sum of the transformed digits. For example, the signal 011011100 has a signal strength of 5.

If binary signals exist in protein sequences then we expected to find linguistic structure in the sequences. One way to detect such structure is to compare the actual signal strength distribution with the expected distribution if protein sequences were random. For a given sequence window length, N_{w}, we scanned our sequence database to determine the distribution of the N_{W}+1 signal strength values. We then used the binomial distribution to compute the signal strength frequencies in random protein sequences. The binomial distribution is a function of N_{w} and the abundance of the selected amino acids, fₐₐ. For the ARQELKM amino acid set, fₐₐ is 0.397 in our collection of 790 protein sequences. FIG. 1 shows the actual and random signal strength distributions for the ARQELKM amino acid set.

FIG. 1 shows that the amino acids ARQELKM tend to cluster with respect to random sequences. That is, in a sequence segment of nine amino acids, the ARQELKM amino acids, taken together as group of like monomers, tend to appear more often in either low or high numbers (0-2 and 6-9) and less often in medium numbers (3-5).

We used the χ² test to determine the probability that the observed distribution is drawn from a random parent population. We computed the χ² value over the N_{w}+1 signal strength values in the observed and random distributions. In this case the χ² value was a local maximum in sequence space. Any single substitution, deletion, or addition to this set resulted in a lower χ2 value. The probability that the parent distribution is random is 10⁻⁸⁵⁶. Clearly, this research demonstrated strong evidence of non random signals in protein sequences.

We searched within the sequence signal space for all χ² local maxima. For test sets of up to six amino acids we exhaustively enumerated the space. For test sets with more than six amino acids selected we used two different optimizers. First, we used the results of the exhaustive enumeration as seeds and add or delete amino acids from the test set until a local maximum was reached. Second, we used random test sets of up to 10 amino acids and randomly made single substitution changes in the test set, one at a time, until a local maximum was reached.

We next looked at the statistical significance of specific signals for a given test set. We scanned our sequence database and compared the total number of occurrences of each signal with the expected number of occurrences if the sequences were random. The probability the signal occurs in a random sequence window is:
P(signal)=fₐₐ^{N}^{ₛₛ}(1−fₐₐ) (Equation 1)

The expected number of occurrences, in the collection of 790 protein sequences, of a given signal is then P(signal) multiplied by the number of possible sequence windows. The number of possible windows is equal to the number of residues in the database, Nᵣ, corrected for edge effects:
E(# signals)=P(signal)(Nᵣ−Nₚ(N_{w}−1)) (Equation 2)
where Nₚ is the number of protein sequences. We compared Equation 2, the expected number of occurrences of a signal, with the observed number of occurrences.

Both of our optimization methods for searching for χ² local maxima led to the same results. We found two useful amino acids sets with a χ² local maximum, ARQELKM and CILMFWYV. There also exist two other redundant, identical χ² local maxima corresponding to the respective complementary amino acids sets. FIG. 2 shows the actual and random signal strength distributions for the useful CILMFWYV amino acid set.

FIG. 2 shows that the CILMFWYV amino acid set tends to anticluster with respect to random sequences. The set has lower frequencies in the extreme signal strength values (0-1 and 5-9) and higher frequencies in the middle signal strength values (2-4). In this case the χ² value is 5,173 and the probability that the parent distribution is random is 10⁻¹¹¹⁴.

2. Signal Frequency

The signal 001100100 for the CILMFWYV amino acid set is a statistically significant signal as it occurs 801 times in our database but would be expected to occur only 479 times in random sequences of equal length, according to Equation 2. The signal frequency is therefore 801/479, or 1.67. The signal frequency may be sub or super unity, and statistically significant signals may have low or high frequencies. For this reason it is also useful to compute the corresponding sequence χ² value. This single category in the χ² calculation is a useful metric of the statistical significance of the signal's occurrences in actual protein sequences. For this signal the sequence χ² value is 216.3.

3. Correlating Signals with Local Structure

Another property of a signal is its correlation with local structure. We used a library of 28 representative fragments that span the space of local structure. The fragments are alpha helix, beta strand, beta turn, turn+beta, helix+turn, helix cap, extended helix, Gly/Pro twist, beta+turn, helix-hairpin, beta cap, helix hairpin, beta hairpin, contorted helix, turn, helix+turn II and helix turn, as well as others (see Hunter, (2003) Proteins: Struct., Funct. and Gen. 50, 580; Hunter (2003) Proteins. Mar 1;50(4): 580-8; Hunter, (2003) Proteins. Mar 1;50(4): 572-9; and Cornelius George Hunter, Protein Structure Analysis and Prediction, UMI Dissertation Services, Ann Arbor, Mich. (2001)). We compared the fragment frequencies, associated with certain signals, generated using Class 2 amino acids, with the overall fragment frequencies in the database. In this case there were 28 centroids considered. Though no structural information was used to identify these signals, they are strongly correlated with secondary structure. A summary of this data is shown in Tables 16 and 17

FIG. 3 plots these characteristics for all the signals in the class 1 signal class. Except where noted, this and all following results are based on nine-residue windows. Five of the class 1 amino acids are known to be correlated with the helix secondary structure and the helix propensity of this class is evident in FIG. 3D.

FIG. 4 plots these characteristics for all Class 2 signals. FIGS. 3 and 4 reveal marked trends in the characteristics of the two signal classes. This includes significant correlations with local structure though no structural information was used in identifying the signals. FIGS. 3A and 4A, for example, show that in both signal classes the structural propensity of the signal (local structure χ² value) is strongly correlated with the statistical significance of the signal (sequence χ² value). The farther from random that a signal is, the greater its correlation with local structure is likely to be. Table 1 summarizes the two signal classes identified as useful sets of amino acids.

4. Identifying coding regions

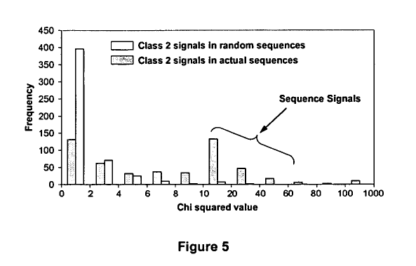

One application for protein sequence signals is in the problem of gene recognition. (see Thayer, (2000) J. Comput. Biol. 7, 317). One way to recognize protein coding DNA regions from non coding regions is to use sequence discriminants. Protein signals are useful in discriminating coding from non coding regions. For example, FIG. 5 shows the distribution of the class 2 signal sequence χ² values in actual and random sequences. Whereas random sequences have very few signals with sequence χ² values greater than 8, the actual sequences contain hundreds of them. The probability that a reading frame represents a protein coding region is evaluated by examining its class 1 and class 2 sequence signal content, as discussed in earlier sections.

5. Comparative Analysis of Multiple Proteins and Structure Prediction

Another application for protein sequence signals is in comparative analysis of multiple proteins to identify conserved signal patterns and predict fold. We found that the number of conserved signals among proteins with the same fold is greater than would be expected if the sequence differences were random.

We computed the expected number of conserved signals in a collection of Nₛ sequences that are aligned with a query sequence, assuming their differences are random. First, for a given sequence similarity value, or identity fraction, f₅, between two sequences, the probability that a residue switches from being in the signal class to being out of the signal class, or vice-versa, is:
Pₛ=2(1−fₛ)fₐₐ(1fₐₐ) (Equation 3)

Then for a given window length, N_{w}, and number of non overlapping signals in the query sequence, N_{signal}, the expected number of conserved signals, N_{cs}, is:
N_{cs}=N_{signals}(1−Pₛ)^{N}^{_{w}}^{N} (Equation 4)

For example, we used the human hemoglobin alpha chain as our query (PDB code: 2hhbA). The sequence contains 82 class 2 statistically-significant signals (those with sequence χ² 10 or more, see FIG. 5). Most of these signals are overlapping. The number of non overlapping signals is 12. For class 2 the value of fₐₐ is 0.339. We compared this sequence with two other hemoglobin sequences from the sickling deer and graylag goose (PDB codes: 1hdsC and 1fawC, respectively). Standard BLAST alignments produce sequence identities of 77% and 70% (fₛ values of 0.77 and 0.70) to 2hhbA, respectively, and 62% between them (see Tatusova, (1999) FEMS Microbiol. Lett. 174, 247).

Equation 2 indicates we should expect between one and two common signals to be conserved in both 1hdsC and 1fawC. Similarly, we simulated the process using random substitutions and found two common signals. The actual signal sequences, however, contain eight conserved non overlapping signals. These are highlighted in the signal sequences given in Table 2.

These results are typical. We have studied many hemoglobin and beta barrel sequences and we consistently find more conserved signals that would be expected from random substitutions.

We have found two signal classes in protein sequences which together contain a total of 300 statistically significant signals. Though we have used a purely sequence-based approach, the signal classes are chemically rational (class 2 consists of the eight most hydrophobic amino acids), and the signals correlate with local and tertiary structure.

### EXAMPLE 2

Protein sequences contain a large number of statistically significant signals, signals which are highly unlikely to be there by chance. We investigated the relationship between the protein signals in a sequence and the corresponding fold for which the sequence codes. We showed that the signals can be used to predict the fold with very high accuracy rates (99.6% in a 794-protein training set and 100% in a 30-protein test set).

We assembled a database of protein amino acid sequences from six different protein families. To avoid highly redundant sequences we filtered using a ≧20%-≦90% sequence identity threshold. This threshold means that all members of the family used in the analysis have greater than 20% amino acid identity with all other members of the family but no member of the family has greater than 90% amino acid sequence identity with any other member of the family. The database identifier codes for each sequence are listed in Tables 7 through 13.

To use the sequence signals to predict fold, we considered both the rate at which signals occur in protein families, and the locations at which they occur in the sequence. Table 5 shows typical occurrence rates of signals in our non redundant database and the six protein families. The signals in Table 5 show that the occurrence rates for a given signal may vary by more than an order of magnitude across different protein families. For example, Signal 92 in class 2 occurs almost twice as often in globin sequences as would be expected if signals were not correlated with family (13.63 occurrences per 1000 loci in the globins as compared to 7.50 occurrences per 1000 loci in our non redundant database). Yet this same signal has only 0.17 occurrences per 1000 loci in the monoclonal antibody sequences.

Similarly, FIGS. 6 and 7 show how the location of signal 92 varies between protein families. FIG. 6 shows that in the globins, signal 92 has a strong tendency to occur just after the half-way point. FIG. 7 shows that signal 92 most often occurs late in the sequence in thioredoxin proteins.

We used the raw location data, such as those plotted in the FIGS. 6 and 7 histograms, to estimate the signal location probability density function (PDF). We use the method based on the fast Fourier transform (FFT) advanced by Silverman (see Silverman, Applied Statistics, 31: 93-99, 1982; Silverman, in Density Estimation for Statistics and Data Analysis, London: Chapman and Hall, pp. 61-66, 1986; and Breipohl, Probabilistic Systems Analysis, New York: John Wiley & Sons, p. 34, 1970). This method computes Gaussian kernel estimates of a univariate density using the FFT over a fixed kernel interval. As such, one of the key parameters determining the smoothness of the resulting PDF is the kernel width value. We found that a kernel width value of 0.05 produces good PDFs.

Smaller kernel widths did not provide smooth results and instead fit the raw data too tightly. This resulted in PDFs that adhere too closely to the particulars of the raw data rather than modeling the tendencies of the protein family. Conversely, larger kernel widths made for overly smooth results that failed to model the important trends of the protein family. FIG. 8 plots together the two PDFs from FIGS. 6 and 7. This comparison illustrates the significant differences between the location tendencies of signal 92 in these two families. This level of difference is not unusual, and it provides a strong discrimination against false folds.

We used Bayes' rule (see Silverman references, above) to judge the likelihood that a given sequence of signals codes for a given fold:

$$P ( A \vert B ) = \frac{P ( B \vert A )}{P ( B )} P ( A ) \qquad ( Equation 5 )$$

where P(A) is the prior probability of event A, P(B) is the probability of event B, and P(B|A) is the probability of event B given event A. For our predictor, event A was a hypothesized fold, Fᵢ (i.e., the event that the sequence in question codes for fold Fᵢ), and event B was the occurrence of signal j at loci k in the sequence, Sⱼₖ, so that:

$$P ( {F}_{i} \vert {S}_{jk} ) = \frac{P ( {S}_{jk} \vert {F}_{i} )}{P ( {S}_{jk} )} P ( {F}_{i} ) \qquad ( Equation 6 )$$

We applied Equation 6 iteratively for each signal in the sequence. In iteration m we updated P(Fᵢ) for the subsequent iteration, m+1:
P₍Fᵢ)ₘ₊₁=P(Fᵢ|Sⱼₖ)ₘ (Equation 7)

In general, the signal occurrences across a sequence were correlated. That is, for a given protein family, Sⱼₖ may be correlated with Sᵣₛ even for loci, k and s, that are distant in the sequence. Therefore, the iterative use of Equation 6 includes non independent factors, and so the result is not a true probability. Instead, it is a fold figure of merit, or fold score.

For rarely occurring signals there is little raw location data. In this case there is less confidence that the estimated PDF reflects a true trend within the protein family. Therefore, we used a threshold of 20 occurrences. If a signal occurs 20 times or less in a protein family, then we collapsed the PDF so that the signal location was not considered:

$$P ( {F}_{i} \vert {S}_{j} ) = \frac{P ( {S}_{j} \vert {F}_{i} )}{P ( {S}_{j} )} P ( {F}_{i} ) \qquad ( Equation 8 )$$

As shown in Table 4, we have collected 824 sequences from six different protein families. We randomly selected five sequences from each family. We used these 30 sequences as test cases. We used the remaining 794 sequences to construct the P(Sⱼₖ|Fᵢ) and P(Sⱼ|Fᵢ) terms in Equations 6 and 8, respectively. These terms were computed for each of the six protein families. This process was done for each of the two signal classes. Therefore, for a given query sequence, we computed a total of 12 fold scores, for each of the six families and two signal classes.

For a given query sequence, we used our fold predictor to compute fold scores. We computed scores for both the training set and the test set sequences. Table 6 summarizes the results, and shows that the protein sequence signals are powerful fold discriminators. In the previous example, we found that the class 2 signals were of higher statistical significance and had greater local structure correlation than the class 1 signals. Therefore it was not surprising that they also performed slightly better in fold prediction. Table 6 shows that the class 2 fold scores provide near perfect fold prediction.

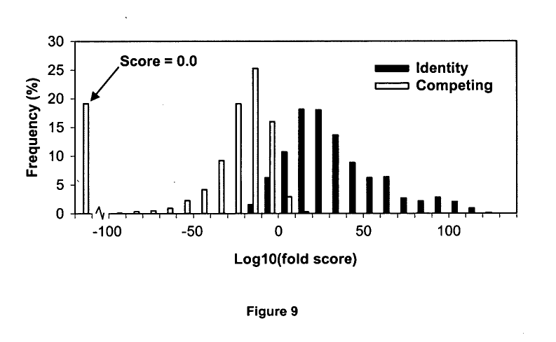

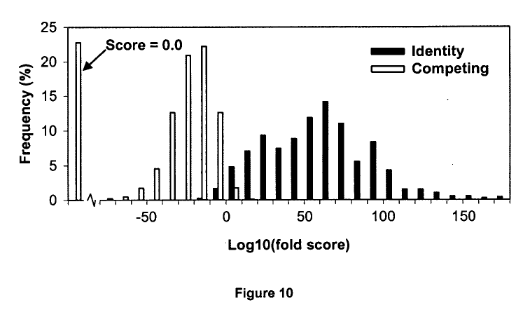

In general, identity fold scores (the score of the fold that the sequence codes for) are many orders of magnitude greater than the competing scores. FIGS. 9 and 10 show the distributions of the identity and competing scores for the class 1 and 2 signal predictors, respectively.

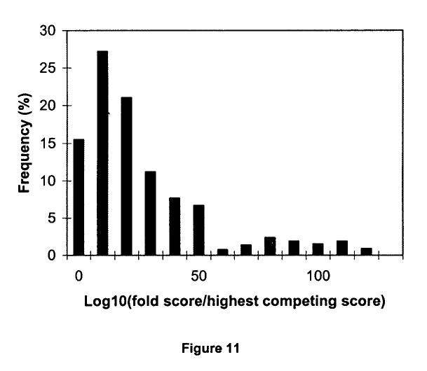

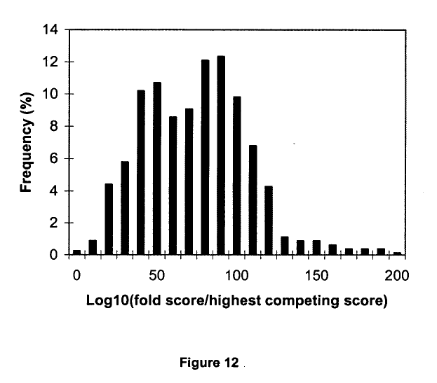

Both the identity and competing distributions included a wide range of values. For a given query sequence, however its fold scores were highly correlated. That is, if a query sequence has a low identity score then it will likely have low competing scores as well. Likewise, if the query sequence has a high identity score then it will likely have high competing scores. FIGS. 11 and 12 show the ratio of the identity score to the highest competing score for the class 1 and 2 predictors, respectively. These plots show that the identity score is typically many orders of magnitude greater than even the highest competing score.

The above description and Examples are illustrative and not restrictive. Many variations of the invention will become apparent to those of skill in the art upon review of this disclosure. Merely by way of example, while the invention is illustrated primarily with regard to signal analysis, the invention is not so limited. The scope of the invention should, therefore, be determined not with reference to the above description, but instead should be determined with reference to the appended claims along with their full scope of equivalents.

All patent filings and publications cited herein are incorporated herewith by reference for the purposes to the same extent as if each were so individually described.

TABLE 1
Characteristics of two useful amino acid sets.

| | | #Signals of | Pattern |
|---|---|---|---|
| Signal class | Amino acids | x² > 10 | distribution |
| 1 | ARQELKM | 84 | clustering |
| 2 | CILMFWYV | 216 | anticlustering |

TABLE 2
Class 2 signal designations for three distinct hemoglobin proteins. Highlighted
segments show a significant number of aligned signals (35 signals in 8 separate segments). With
random sequence divergence only one or two conserved signals are expected.

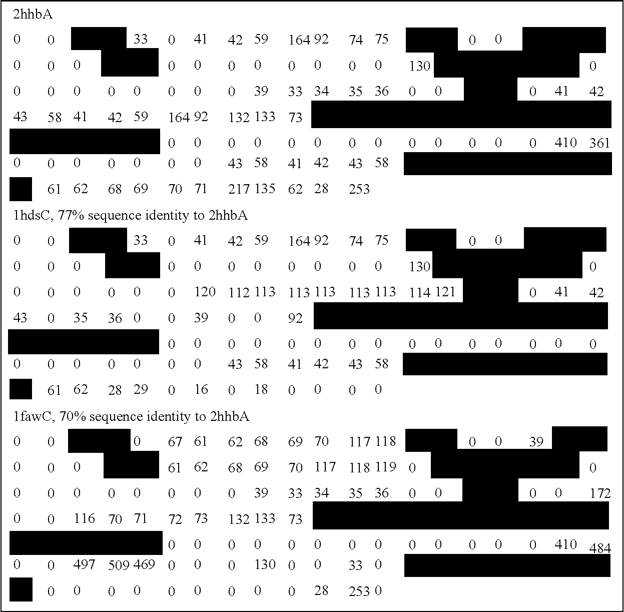

TABLE 3
A collection of proteins sequences used in the described methods. The 790
protein data bank PDB sequences were used in Examples 1 and 2.

| | | | | | | | | | |
|---|---|---|---|---|---|---|---|---|---|
| 119l_{—} | 153l_{—} | 16pk_{—} | 1a02N | 1a0aA | 1a15B | 1a17_{—} | 1a1iA | 1alx_{—} | 1a26_{—} |
| 1a28A | 1a2pA | 1a2yB | 1a2zA | 1a34A | 1a4mA | 1a4sA | 1a68_{—} | 1a73A | 1a7i_{—} |
| 1a7tA | 1a8d_{—} | 1a8e_{—} | 1a9s_{—} | 1aa0_{—} | 1aa7A | 1ab7_{—} | 1aba_{—} | 1abrB | 1acp_{—} |
| 1acz_{—} | 1ad2_{—} | 1ad6_{—} | 1adoA | 1ads_{—} | 1afp_{—} | 1afrA | 1ag4_{—} | 1agg_{—} | 1agjA |
| 1agnA | 1agqD | 1agrH | 1ah1_{—} | 1ah7_{—} | 1ah9_{—} | 1ahjA | 1ahjB | 1ahk_{—} | 1ahsA |
| 1ai7A | 1aie_{—} | 1aijS | 1aikC | 1ai1_{—} | 1aj2_{—} | 1aj3_{—} | 1ajj_{—} | 1ak0_{—} | 1ak1_{—} |
| 1ak4C | 1ako_{—} | 1akz_{—} | 1alo_{—} | 1alvA | 1aly_{—} | 1amm_{—} | 1amp_{—} | 1amx_{—} | 1an2A |
| 1an7A | 1an8_{—} | 1an9A | 1anf_{—} | 1ao6A | 1aocA | 1aohB | 1aoiF | 1aoiG | 1aojA |
| 1aol_{—} | 1aonO | 1aoo_{—} | 1aoqA | 1aorA | 1aoy_{—} | 1aozA | 1ap0_{—} | 1ap8_{—} | 1apf_{—} |
| 1apj_{—} | 1apyB | 1aq0A | 1aq6A | 1aqb_{—} | 1ar0A | 1ar1A | 1arb_{—} | 1ark_{—} | 1arv_{—} |
| 1arzC | 1as4B | 1ash_{—} | 1asx_{—} | 1asyA | 1atg_{—} | 1at1A | 1atzA | 1au1A | 1aua_{—} |
| 1aurA | 1avmA | 1avoB | 1aw8E | 1awd_{—} | 1awj_{—} | 1awo_{—} | 1ax3_{—} | 1axn_{—} | 1axwA |
| 1ayoA | 1ayyA | 1b0m_{—} | 1b10_{—} | 1b2nA | 1b2nB | 1bak_{—} | 1baq_{—} | 1bazC | 1bbpA |
| 1bbxC | 1bc4_{—} | 1bc5A | 1bc8C | 1bcfA | 1bcn_{—} | 1bcpC | 1bct_{—} | 1bd0A | 1bd8_{—} |
| 1bdyA | 1be1_{—} | 1bea_{—} | 1bebA | 1behA | 1benB | 1beo_{—} | 1bev1 | 1bf8_{—} | 1bfd_{—} |
| 1bfeA | 1bfg_{—} | 1bg0_{—} | 1bg2_{—} | 1bg8A | 1bgf_{—} | 1bgp_{—} | 1bh5B | 1bisB | 1bjk_{—} |
| 1bkf_{—} | 1bkrA | 1b10A | 1ble_{—} | 1bndB | 1bnkA | 1bnlA | 1bo4B | 1bol_{—} | 1bor_{—} |
| 1bovA | 1bp1_{—} | 1bq3B | 1bqhI | 1bquB | 1br0_{—} | 1brf_{—} | 1brt_{—} | 1bsn_{—} | 1btkB |
| 1btmA | 1btn_{—} | 1bu7A | 1buoA | 1buz_{—} | 1bvh_{—} | 1bw3_{—} | 1bxa_{—} | 1byb | 1bym_{—} |
| 1byqA | 1c25_{—} | 1c3d_{—} | 1c52_{—} | 1c5a_{—} | 1cawB | 1cby_{—} | 1cd1A | 1cdb_{—} | 1cdi_{—} |
| 1cem_{—} | 1cewI | 1cex_{—} | 1cfb_{—} | 1cfe_{—} | 1cfh_{—} | 1cfr_{—} | 1cfyA | 1cg2A | 1chd_{—} |
| 1chkA | 1chl_{—} | 1chmA | 1cid_{—} | 1ckaA | 1cknA | 1clc_{—} | 1cmkE | 1cmyB | 1cne_{—} |
| 1cnv_{—} | 1cp2A | 1cpcB | 1cpo_{—} | 1crxA | 1csbB | 1cseI | 1csgA | 1csh_{—} | 1csn_{—} |
| 1cto_{—} | 1cur_{—} | 1cydA | 1cyo_{—} | 1cyx_{—} | 1d2nA | 1d66A | 1dad_{—} | 1ddf_{—} | 1deaA |
| 1dec_{—} | 1def_{—} | 1dfjI | 1dfx_{—} | 1dhr_{—} | 1div_{—} | 1dktB | 1dkzA | 1dlc_{—} | 1dlhB |
| 1dpsB | 1dupA | 1dxy_{—} | 1e2aA | 1eal_{—} | 1ebpA | 1eca_{—} | 1eceA | 1ecpA | 1ecrA |
| 1edg_{—} | 1edmB | 1edt_{—} | 1efvA | 1efvB | 1ehs_{—} | 1elyA | 1erd_{—} | 1erv_{—} | 1esc_{—} |
| 1etpA | 1euu_{—} | 1exg_{—} | 1ezm_{—} | 1fbr_{—} | 1fc1A | 1fcdA | 1fdzB | 1fgjA | 1fleI |
| 1fna_{—} | 1frvB | 1fssA | 1ft1A | 1ft1B | 1ftpA | 1ftrA | 1fts_{—} | 1fua_{—} | 1fuiA |
| 1furA | 1fus_{—} | 1fvkA | 1fvpA | 1fwcA | 1fzaB | 1fzcA | 1g31A | 1gd1O | 1gdoA |
| 1gifA | 1gky_{—} | 1gnhA | 1goh_{—} | 1gotB | 1gotG | 1gpl_{—} | 1gps_{—} | 1grx_{—} | 1gsa_{—} |
| 1gtqA | 1guqB | 1guxB | 1gvp_{—} | 1havA | 1hcd_{—} | 1hcnA | 1hcnB | 1hcrA | 1hdeA |
| 1hev_{—} | 1hfc_{—} | 1hfh_{—} | 1hjrA | 1hkbA | 1hlb_{—} | 1hoe_{—} | 1hpcA | 1hqi_{—} | 1hrdA |
| 1hsbA | 1htrP | 1hulA | 1hxn_{—} | 1iakA | 1ibcA | 1ibcB | 1idaA | 1idk_{—} | 1ido_{—} |
| 1if1b | 1ife_{—} | 1ihfA | 1iibA | 1imdA | 1inp_{—} | 1ipsA | 1irk_{—} | 1irl_{—} | 1irsA |
| 1iso_{—} | 1isuA | 1itbB | 1ixh_{—} | 1jacA | 1jdw_{—} | 1jer_{—} | 1jetA | 1jfrA | 1jhgA |
| 1jkw_{—} | 1jli_{—} | 1jlyA | 1jmcA | 1jpc_{—} | 1jrhI | 1jsuC | 1juk_{—} | 1jvr_{—} | 1jxpA |
| 1kb5B | 1kbs_{—} | 1kid_{—} | 1kigL | 1kit_{—} | 1knb_{—} | 1knyA | 1kpf_{—} | 1kptA | 1krt_{—} |
| 1ksr_{—} | 1kte_{—} | 1kuh_{—} | 1kveA | 1kveB | 1kvu_{—} | 1kwaA | 1kzuB | 1lam_{—} | 1latA |
| 1lba_{—} | 1lcl_{—} | 1leb_{—} | 1lghA | 1lki_{—} | 1lkkA | 1lktA | 1lmb3 | 1lou_{—} | 1lpbA |
| 1lrv_{—} | 1ltsA | 1lxa_{—} | 1lxtA | 1mai_{—} | 1mak_{—} | 1mbl_{—} | 1mbh_{—} | 1mbj_{—} | 1mkaA |
| 1mldA | 1mml_{—} | 1mnmC | 1molA | 1moq_{—} | 1mpgA | 1mrj_{—} | 1mroB | 1mroC | 1msc_{—} |
| 1msi_{—} | 1msk_{—} | 1mspB | 1mtyB | 1mtyD | 1mtyG | 1mugA | 1mup_{—} | 1mut_{—} | 1mypA |
| 1mzm_{—} | 1nar_{—} | 1nbaB | 1nbbA | 1nbcA | 1nciA | 1nfdA | 1ngr_{—} | 1nif_{—} | 1nkl_{—} |
| 1nkr_{—} | 1nksA | 1nls_{—} | 1noe_{—} | 1nox_{—} | 1noyA | 1np4_{—} | 1npk_{—} | 1npoC | 1nsgB |
| 1nwpA | 1oakA | 1occC | 1occD | 1occE | 1occF | 1occG | 1occH | 1occK | 1ocp_{—} |
| 1ofgA | 1onrA | 1opd_{—} | 1opr_{—} | 1orc_{—} | 1ospO | 1otgA | 1otp_{—} | 1oyc_{—} | 1p04A |
| 1pboB | 1pbwB | 1pce_{—} | 1pdnC | 1pdo_{—} | 1pea_{—} | 1pex_{—} | 1pfsA | 1pft_{—} | 1pgs_{—} |
| 1phc_{—} | 1phnA | 1pih_{—} | 1pioA | 1pkp_{—} | 1plr_{—} | 1pmi_{—} | 1pne_{—} | 1pnkB | 1poa_{—} |
| 1poc_{—} | 1poiA | 1poiB | 1pot_{—} | 1pou_{—} | 1ppn_{—} | 1ppt_{—} | 1prcC | 1prtF | 1pty_{—} |
| 1pud_{—} | 1put_{—} | 1pyaB | 1pyp_{—} | 1pysA | 1pytA | 1qapA | 1qba_{—} | 1qnf_{—} | 1qyp_{—} |
| 1ra9_{—} | 1rcf_{—} | 1regX | 1reqB | 1ret_{—} | 1rfbA | 1rgeA | 1rgs_{—} | 1rie_{—} | 1rlaA |
| 1rlw_{—} | 1rmd_{—} | 1rmg_{—} | 1rof_{—} | 1rpo_{—} | 1rpt_{—} | 1rsy_{—} | 1rtoA | 1rvaA | 1ryp1 |
| 1ryp2 | 1rypF | 1rypI | 1sbp_{—} | 1scmA | 1sco_{—} | 1sfcA | 1sfcB | 1sfcD | 1sfe_{—} |
| 1sfp_{—} | 1sgpI | 1shcA | 1skyE | 1skz_{—} | 1sltB | 1sly_{—} | 1smd_{—} | 1smeA | 1smnA |
| 1smpI | 1smtB | 1smvC | 1spy_{—} | 1sqc_{—} | 1sra_{—} | 1sro_{—} | 1std_{—} | 1stfI | 1stmA |
| 1svb_{—} | 1svpA | 1svr_{—} | 1tadA | 1tafA | 1tafB | 1tahA | 1tam_{—} | 1tbn_{—} | 1tc3C |
| 1tca_{—} | 1tde_{—} | 1tfb_{—} | 1tfe_{—} | 1tfpA | 1thjA | 1thv_{—} | 1tib_{—} | 1tih_{—} | 1tiiD |
| 1tit_{—} | 1tiv_{—} | 1tkaA | 1tle_{—} | 1tme1 | 1tml_{—} | 1tnrA | 1tpn_{—} | 1tsg_{—} | 1tul_{—} |
| 1tupA | 1tvxB | 1tx4A | 1tyfA | 1uae_{—} | 1ubi_{—} | 1uby_{—} | 1udiI | 1ueaB | 1ulo_{—} |
| 1unkA | 1uroA | 1utg_{—} | 1uxd_{—} | 1uxy_{—} | 1vcaA | 1vcc_{—} | 1vdfA | 1vhh_{—} | 1vhrA |
| 1vid_{—} | 1vif_{—} | 1vig_{—} | 1vin_{—} | 1vkxB | 1v1s_{—} | 1vmoA | 1vpsB | 1vsd_{—} | 1vtx_{—} |
| 1wab_{—} | 1wdcB | 1wer_{—} | 1whi_{—} | 1who_{—} | 1whtB | 1wiu_{—} | 1wkt_{—} | 1wtuA | 1xbrA |
| 1xdtR | 1xgsA | 1xikA | 1xnb_{—} | 1xsoA | 1xtcC | 1xvaA | 1xxaB | 1xyzA | 1yaiA |
| 1yasA | 1ycc_{—} | 1ycqA | 1ycsB | 1yrnA | 1ysc_{—} | 1ystH | 1ytbA | 1ytfC | 1yua_{—} |
| 1yub_{—} | 1yveI | 1zaq_{—} | 1zid_{—} | 1zin_{—} | 1zmeC | 1zug_{—} | 1zwa_{—} | 1zxq_{—} | 256bA |
| 2a0b_{—} | 2abd_{—} | 2abk_{—} | 2acy_{—} | 2adx_{—} | 2ayh_{—} | 2baa_{—} | 2bb8_{—} | 2bbkH | 2bbkL |
| 2bbvA | 2bby_{—} | 2bds_{—} | 2bopA | 2bpa1 | 2bpa2 | 2brz_{—} | 2cba_{—} | 2ccyA | 2chsA |
| 2cps_{—} | 2ctc_{—} | 2cyp_{—} | 2dorA | 2dpg_{—} | 2dri_{—} | 2drpD | 2dynA | 2ech_{—} | 2eiaA |
| 2end_{—} | 2erl_{—} | 2ezh_{—} | 2ezl_{—} | 2fha_{—} | 2fivA | 2fn2 | 2fow_{—} | 2frvA | 2fsp_{—} |
| 2gdm_{—} | 2hbg_{—} | 2hfh_{—} | 2hgf_{—} | 2hoa_{—} | 2hp8_{—} | 2hqi_{—} | 2i1b_{—} | 2igd_{—} | 2ilk_{—} |
| 2izhB | 2lfb_{—} | 2liv_{—} | 2masA | 2mcm_{—} | 2mev4 | 2msbA | 2mtaC | 2nacA | 2nef_{—} |
| 2new_{—} | 2omf_{—} | 2pac_{—} | 2pgd_{—} | 2phy_{—} | 2pia_{—} | 2pii_{—} | 2plc_{—} | 2pldA | 2polA |
| 2por_{—} | 2pspA | 2pth_{—} | 2ptl_{—} | 2pvb_{—} | 2qwc_{—} | 2rgf_{—} | 2rn2_{—} | 2rslC | 2sak_{—} |
| 2scpA | 2sicI | 2sn3_{—} | 2sns_{—} | 2spcA | 2stv_{—} | 2sxl_{—} | 2tbd_{—} | 2tgi_{—} | 2thiA |
| 2ucz_{—} | 2vaoA | 2vgh_{—} | 2vil_{—} | 2viuA | 2viuB | 2vpfH | 3bbg_{—} | 3chbD | 3chy_{—} |
| 3cla_{—} | 3cyr_{—} | 3daaA | 3gcb_{—} | 3grs_{—} | 3gsaA | 3mddA | 3minB | 3nll_{—} | 3pbgA |
| 3pte_{—} | 3pviA | 3r1rA | 3sdhA | 3seb_{—} | 3tdt_{—} | 3vub_{—} | 4mt2_{—} | 4pgaA | 5hpgA |
| 5p21_{—} | 5pti_{—} | 6cel_{—} | 6gsvA | 6mhtA | 6pfkA | 7ahlA | 7at1B | 7rsa_{—} | 8abp_{—} |

TABLE 4
Summary of sequences collected for six protein families.

| | | | | | Monoclonal | Amido |
|---|---|---|---|---|---|---|
| | Globin | Lysozyme | Thioredoxin | Trypsin | antibody | transferase |
| Source | Swiss Prot | Swiss Prot | Swiss Prot | Swiss Prot | NCBI | NCBI |
| #Sequences | 426 | 60 | 164 | 52 | 53 | 69 |
| Nmin | 130 | 120 | 100 | 210 | 105 | 165 |
| Nmax | 160 | 180 | 115 | 250 | 125 | 210 |

TABLE 5
Sample signal occurrence rate data in the collection of of 790 non redundant proteins
(DB) and the six protein families listed in tables 7-13. Occurrence rate data
are in terms of number of occurrences per 1000 loci. Data show that the occurrence
rate for a given signal can vary by an order or magnitude. These signals are useful
for fold prediction.

| | | | | | | | | Monoclonal | Amido |
|---|---|---|---|---|---|---|---|---|---|
| Sig # | Signal | Class | DB | Globin | Lysozyme | Thioredoxin | Trypsin | antibody | transferase |
| 92 | 000100100 | 2 | 7.50 | 13.63 | 5.73 | 7.62 | 1.48 | 0.17 | 8.30 |
| 101 | 100001000 | 2 | 5.62 | 3.96 | 5.36 | 6.13 | 7.55 | 1.05 | 3.20 |
| 263 | 000100010 | 1 | 4.62 | 3.02 | 9.14 | 3.03 | 5.50 | 12.58 | 6.32 |
| 26 | 000001101 | 1 | 2.99 | 2.12 | 0.73 | 5.02 | 2.79 | 1.40 | 3.12 |

TABLE 6
Summary of fold predictor performance. For a given query sequence
and signal class, six fold scores are computed for each of the six
protein families. If the highest score corresponds to the correct fold
(i.e., the identity fold), then it is judged to be a correct prediction.

| | | | #Correct | |
|---|---|---|---|---|
| | #Sequences | Signal class | predictions | % Correct |
| Training set | 794 | 1 | 765 | 96.3 |
| Test set | 30 | 1 | 28 | 93.3 |
| Training set | 794 | 2 | 791 | 99.6 |
| Test set | 30 | 2 | 30 | 100.0 |

TABLE 7
Header information for 421 globin sequences used in the fold recognition
analysis. Sequences obtained from Swiss-Prot database.

| | | | | |
|---|---|---|---|---|
| SQ | SEQUENCE | 146 AA; | 15856 MW; | E7FE4DC4D7752254 CRC64; |
| SQ | SEQUENCE | 147 AA; | 16553 MW; | 85067F2447C5089C CRC64; |
| SQ | SEQUENCE | 144 AA; | 15733 MW; | C0CED8B76BF38983 CRC64; |
| SQ | SEQUENCE | 158 AA; | 17011 MW; | 9639E8A38908B8AB CRC64; |
| SQ | SEQUENCE | 147 AA; | 14902 MW; | 980558C06D881C43 CRC64; |
| SQ | SEQUENCE | 142 AA; | 14772 MW; | 49A374E71EA6B6C5 CRC64; |
| SQ | SEQUENCE | 142 AA; | 16129 MW; | 87BE8C74D1BBF1BE CRC64; |
| SQ | SEQUENCE | 149 AA; | 16536 MW; | A1A68F0546F5E88E CRC64; |
| SQ | SEQUENCE | 157 AA; | 17584 MW; | 3FD1F7F8767EC988 CRC64; |
| SQ | SEQUENCE | 141 AA; | 16294 MW; | AD73E09A11B6ED2A CRC64; |
| SQ | SEQUENCE | 139 AA; | 15705 MW; | 02396BED2FD1A2E6 CRC64; |
| SQ | SEQUENCE | 146 AA; | 15995 MW; | 1D61233F70752D1A CRC64; |
| SQ | SEQUENCE | 153 AA; | 17454 MW; | 1B3EF94A15B49B98 CRC64; |
| SQ | SEQUENCE | 136 AA; | 15229 MW; | 8B7C2AB9DDA99D33 CRC64; |
| SQ | SEQUENCE | 150 AA; | 16879 MW; | F74EC930A7807D56 CRC64; |
| SQ | SEQUENCE | 145 AA; | 16254 MW; | 5F62E08A11AA52A6 CRC64; |
| SQ | SEQUENCE | 154 AA; | 17363 MW; | 76FD023645C4F2E1 CRC64; |
| SQ | SEQUENCE | 149 AA; | 16311 MW; | DDD300F482DAD74E CRC64; |
| SQ | SEQUENCE | 146 AA; | 16602 MW; | 67E74FB39BD351E9 CRC64; |
| SQ | SEQUENCE | 151 AA; | 16348 MW; | 19EF29594AEF9FFB CRC64; |
| SQ | SEQUENCE | 144 AA; | 16004 MW; | 036CC2E9B1EF7E69 CRC64; |
| SQ | SEQUENCE | 152 AA; | 17429 MW; | E1B84F4BD8F1D7F2 CRC64; |
| SQ | SEQUENCE | 149 AA; | 16508 MW; | 815802E04F8EE666 CRC64; |
| SQ | SEQUENCE | 148 AA; | 16648 MW; | CF303F4596861B5A CRC64; |
| SQ | SEQUENCE | 148 AA; | 16680 MW; | 40DD2369EAD054D8 CRC64; |
| SQ | SEQUENCE | 151 AA; | 15935 MW; | 7D5CA0B3554D01AF CRC64; |
| SQ | SEQUENCE | 151 AA; | 17525 MW; | 4A2C7421F0DCBC2F CRC64; |
| SQ | SEQUENCE | 149 AA; | 16795 MW; | 6567417159F70C4D CRC64; |
| SQ | SEQUENCE | 152 AA; | 17074 MW; | 6F7FDB2AEFB28A8D CRC64; |
| SQ | SEQUENCE | 145 AA; | 15181 MW; | C7B0EC2BB9DF3CD8 CRC64; |
| SQ | SEQUENCE | 151 AA; | 16898 MW; | 3BAFD45225E51B59 CRC64; |
| SQ | SEQUENCE | 150 AA; | 16300 MW; | 882F2EA6587ED42D CRC64; |
| SQ | SEQUENCE | 151 AA; | 16393 MW; | 7CF9C918BEB9FE8C CRC64; |
| SQ | SEQUENCE | 144 AA; | 16135 MW; | 9A094A9E8E981568 CRC64; |
| SQ | SEQUENCE | 158 AA; | 17675 MW; | 363BC16BD9661352 CRC64; |
| SQ | SEQUENCE | 159 AA; | 18485 MW; | 0C2E55AC5B6583FE CRC64; |
| SQ | SEQUENCE | 151 AA; | 16874 MW; | DFF2528851D80CF0 CRC64; |
| SQ | SEQUENCE | 152 AA; | 15964 MW; | 52D70B8CF57CFA9E CRC64; |
| SQ | SEQUENCE | 151 AA; | 16155 MW; | 38DB0DAC4AE64E2E CRC64; |
| SQ | SEQUENCE | 159 AA; | 17999 MW; | 6A688F622B9B9CD3 CRC64; |
| SQ | SEQUENCE | 151 AA; | 17068 MW; | 9C02E8D3001D29AE CRC64; |
| SQ | SEQUENCE | 144 AA; | 15016 MW; | 28FCF0FC578E50FB CRC64; |
| SQ | SEQUENCE | 144 AA; | 15328 MW; | 20F30D6FC1D11554 CRC64; |
| SQ | SEQUENCE | 146 AA; | 15324 MW; | 2AC8E33C3206FC86 CRC64; |
| SQ | SEQUENCE | 146 AA; | 15360 MW; | 034F81969E64DE66 CRC64; |
| SQ | SEQUENCE | 147 AA; | 15749 MW; | 307996E954FA1054 CRC64; |
| SQ | SEQUENCE | 151 AA; | 16210 MW; | 3493BAE8F4A4BD90 CRC64; |
| SQ | SEQUENCE | 146 AA; | 15319 MW; | 08D5EFC0170A0D23 CRC64; |
| SQ | SEQUENCE | 148 AA; | 16517 MW; | C01EBEAD30EB3D3D CRC64; |
| SQ | SEQUENCE | 147 AA; | 15759 MW; | FE2D07817D61CC8C CRC64; |
| SQ | SEQUENCE | 147 AA; | 16639 MW; | BA5062C05B8DEE3D CRC64; |
| SQ | SEQUENCE | 141 AA; | 15922 MW; | 4BA1A1331B0C2A88 CRC64; |
| SQ | SEQUENCE | 147 AA; | 16019 MW; | 20E799D4B18A6718 CRC64; |
| SQ | SEQUENCE | 147 AA; | 15977 MW; | DD3D2C176047BCDB CRC64; |
| SQ | SEQUENCE | 142 AA; | 15591 MW; | 295F7DF997AE3F0F CRC64; |
| SQ | SEQUENCE | 141 AA; | 15367 MW; | 268191D27A2BD136 CRC64; |
| SQ | SEQUENCE | 141 AA; | 15044 MW; | 5A44FABB98551423 CRC64; |
| SQ | SEQUENCE | 141 AA; | 15261 MW; | FE0A826AF71DF850 CRC64; |
| SQ | SEQUENCE | 141 AA; | 15089 MW; | B6AF839562129F8F CRC64; |
| SQ | SEQUENCE | 141 AA; | 15200 MW; | 9D7F46C5C1C0B184 CRC64; |
| SQ | SEQUENCE | 141 AA; | 15439 MW; | 9B56DCCCE5DCBF97 CRC64; |
| SQ | SEQUENCE | 141 AA; | 15271 MW; | CFC2F8EC086EAB60 CRC64; |
| SQ | SEQUENCE | 141 AA; | 15448 MW; | 0B81E2BDE3DF6CBE CRC64; |
| SQ | SEQUENCE | 142 AA; | 15495 MW; | C4BA34C216D2B412 CRC64; |
| SQ | SEQUENCE | 144 AA; | 15306 MW; | DB683115939E78EA CRC64; |
| SQ | SEQUENCE | 141 AA; | 15886 MW; | 36FAFC85D7DD274A CRC64; |
| SQ | SEQUENCE | 141 AA; | 15461 MW; | D8D0FF702686A0F6 CRC64; |
| SQ | SEQUENCE | 141 AA; | 15252 MW; | A6CE1EDA7B722D18 CRC64; |
| SQ | SEQUENCE | 141 AA; | 15866 MW; | 31BA7A1756FC06B7 CRC64; |
| SQ | SEQUENCE | 142 AA; | 15658 MW; | 61AA6B05E0C5DD48 CRC64; |
| SQ | SEQUENCE | 141 AA; | 15614 MW; | 4D55B7E3B080CC95 CRC64; |
| SQ | SEQUENCE | 141 AA; | 15930 MW; | F3535256589083C4 CRC64; |
| SQ | SEQUENCE | 141 AA; | 15720 MW; | 2E4578D61EBFD9FF CRC64; |
| SQ | SEQUENCE | 133 AA; | 14915 MW; | 1FB08E8B994002D5 CRC64; |
| SQ | SEQUENCE | 141 AA; | 15714 MW; | 4059AC571F483ED6 CRC64; |
| SQ | SEQUENCE | 141 AA; | 15785 MW; | 5E19140D555A1758 CRC64; |
| SQ | SEQUENCE | 141 AA; | 15237 MW; | 26DB4610C73E32E9 CRC64; |
| SQ | SEQUENCE | 142 AA; | 15219 MW; | A5F1E38681449C7B CRC64; |
| SQ | SEQUENCE | 141 AA; | 14874 MW; | 7B87E60248EDDD0F CRC64; |
| SQ | SEQUENCE | 141 AA; | 15303 MW; | F0F1694366B7C0A7 CRC64; |
| SQ | SEQUENCE | 142 AA; | 15803 MW; | F23E3258D250F66C CRC64; |
| SQ | SEQUENCE | 141 AA; | 15270 MW; | 73E02EDE6BF6ECEB CRC64; |
| SQ | SEQUENCE | 142 AA; | 15905 MW; | 8C21BE6324D5D586 CRC64; |
| SQ | SEQUENCE | 142 AA; | 15518 MW; | 058246F5463582D6 CRC64; |
| SQ | SEQUENCE | 141 AA; | 15891 MW; | 2E052F26984A7E3F CRC64; |
| SQ | SEQUENCE | 143 AA; | 15816 MW; | D851CF3EA4707A21 CRC64; |
| SQ | SEQUENCE | 142 AA; | 15189 MW; | F61A7B96A07A41CD CRC64; |
| SQ | SEQUENCE | 142 AA; | 15545 MW; | F4139EAE0C7407C9 CRC64; |
| SQ | SEQUENCE | 141 AA; | 15781 MW; | 0F499EC4ECFDBB7D CRC64; |
| SQ | SEQUENCE | 141 AA; | 15746 MW; | 7F23AC9F0170A6EB CRC64; |
| SQ | SEQUENCE | 141 AA; | 15745 MW; | 8C18969F07934ED0 CRC64; |
| SQ | SEQUENCE | 141 AA; | 15767 MW; | FD477EC4E61A9EEE CRC64; |
| SQ | SEQUENCE | 141 AA; | 15695 MW; | 1FE426969B7B5384 CRC64; |
| SQ | SEQUENCE | 141 AA; | 16241 MW; | E4681DF8F8C17F3E CRC64; |
| SQ | SEQUENCE | 140 AA; | 15717 MW; | 2FAC884799A152F9 CRC64; |
| SQ | SEQUENCE | 141 AA; | 16097 MW; | C65CA53BC7060920 CRC64; |
| SQ | SEQUENCE | 141 AA; | 15728 MW; | BB6220406F9E0B90 CRC64; |
| SQ | SEQUENCE | 141 AA; | 15979 MW; | 5D0C08FAB4B42035 CRC64; |
| SQ | SEQUENCE | 141 AA; | 16237 MW; | 25217D16E6C0F844 CRC64; |
| SQ | SEQUENCE | 141 AA; | 15788 MW; | B8A833057DCB96EA CRC64; |
| SQ | SEQUENCE | 141 AA; | 16272 MW; | F5B8E6333C9F9AA1 CRC64; |
| SQ | SEQUENCE | 141 AA; | 15680 MW; | 3860B37CB87A1109 CRC64; |
| SQ | SEQUENCE | 132 AA; | 14391 MW; | 70E36423397430DC CRC64; |
| SQ | SEQUENCE | 141 AA; | 15423 MW; | A6260899CFE651F0 CRC64; |
| SQ | SEQUENCE | 141 AA; | 15376 MW; | BC7F1043C4971CB9 CRC64; |
| SQ | SEQUENCE | 141 AA; | 14932 MW; | A34047DE201BCF28 CRC64; |
| SQ | SEQUENCE | 141 AA; | 15566 MW; | E6638D87B5619087 CRC64; |
| SQ | SEQUENCE | 141 AA; | 15432 MW; | 01117A671F942811 CRC64; |
| SQ | SEQUENCE | 141 AA; | 15506 MW; | 971A9BFCE652293A CRC64; |
| SQ | SEQUENCE | 141 AA; | 16104 MW; | BFAD2416B765C03E CRC64; |
| SQ | SEQUENCE | 141 AA; | 15402 MW; | 79CB4AAE8B7AD0E4 CRC64; |
| SQ | SEQUENCE | 141 AA; | 15482 MW; | FA024577E0D35B1C CRC64; |
| SQ | SEQUENCE | 141 AA; | 15734 MW; | F4A95E87BB7ACD86 CRC64; |
| SQ | SEQUENCE | 142 AA; | 15900 MW; | 73C32A82B82A97FE CRC64; |
| SQ | SEQUENCE | 141 AA; | 15229 MW; | 10B2F10BA8347D7E CRC64; |
| SQ | SEQUENCE | 141 AA; | 15335 MW; | 459A83261D291A9D CRC64; |
| SQ | SEQUENCE | 141 AA; | 15468 MW; | 65AA45765D333866 CRC64; |
| SQ | SEQUENCE | 141 AA; | 14886 MW; | 228D4A8D4832781D CRC64; |
| SQ | SEQUENCE | 141 AA; | 15406 MW; | 0B0C26CDF7B72B53 CRC64; |
| SQ | SEQUENCE | 142 AA; | 15391 MW; | BC152C73231E0797 CRC64; |
| SQ | SEQUENCE | 141 AA; | 15149 MW; | 2808540F975F9435 CRC64; |
| SQ | SEQUENCE | 141 AA; | 15767 MW; | 671B4C10C474238B CRC64; |
| SQ | SEQUENCE | 141 AA; | 15172 MW; | 2E9DB0CF6B676E5C CRC64; |
| SQ | SEQUENCE | 142 AA; | 15426 MW; | 1259F2E3271882CB CRC64; |
| SQ | SEQUENCE | 141 AA; | 15687 MW; | 5ED9D80D430934DD CRC64; |
| SQ | SEQUENCE | 142 AA; | 15431 MW; | 6CE93FDFAF90E451 CRC64; |
| SQ | SEQUENCE | 141 AA; | 15229 MW; | 5E395A8F74D41962 CRC64; |
| SQ | SEQUENCE | 141 AA; | 15124 MW; | 617C52684E6CAAC1 CRC64; |
| SQ | SEQUENCE | 141 AA; | 15303 MW; | 8BDCEA7B8DE0DDB9 CRC64; |
| SQ | SEQUENCE | 141 AA; | 15387 MW; | 1308760ABE73DB21 CRC64; |
| SQ | SEQUENCE | 142 AA; | 15766 MW; | 18F6830D492C9274 CRC64; |
| SQ | SEQUENCE | 141 AA; | 15298 MW; | 77B47DEC96830640 CRC64; |
| SQ | SEQUENCE | 141 AA; | 15642 MW; | 1806FEAC8240F6EE CRC64; |
| SQ | SEQUENCE | 141 AA; | 15467 MW; | EBD30558853D6010 CRC64; |
| SQ | SEQUENCE | 141 AA; | 15141 MW; | 85FE77E89AAFE694 CRC64; |
| SQ | SEQUENCE | 141 AA; | 15048 MW; | B9192AA9050CE540 CRC64; |
| SQ | SEQUENCE | 141 AA; | 15194 MW; | 1D1C17BA24664F51 CRC64; |
| SQ | SEQUENCE | 141 AA; | 15574 MW; | B27FD545835C121C CRC64; |
| SQ | SEQUENCE | 141 AA; | 15152 MW; | 8BE1B3DF84BA7568 CRC64; |
| SQ | SEQUENCE | 142 AA; | 15713 MW; | CA4F1A43C4A17DB7 CRC64; |
| SQ | SEQUENCE | 141 AA; | 15169 MW; | 9AEBD652DC407D97 CRC64; |
| SQ | SEQUENCE | 141 AA; | 15837 MW; | 8BCEEE15F09E116A CRC64; |
| SQ | SEQUENCE | 141 AA; | 15179 MW; | 92684864760E5523 CRC64; |
| SQ | SEQUENCE | 141 AA; | 15156 MW; | 4E007FE421A2A42C CRC64; |
| SQ | SEQUENCE | 141 AA; | 15257 MW; | 509FD1A57AAEDD07 CRC64; |
| SQ | SEQUENCE | 141 AA; | 15027 MW; | 379F8241EC1E9D29 CRC64; |
| SQ | SEQUENCE | 142 AA; | 15344 MW; | 1A4139F77ABFF734 CRC64; |
| SQ | SEQUENCE | 141 AA; | 15530 MW; | 3EAC8AEAAEECA0F0 CRC64; |
| SQ | SEQUENCE | 141 AA; | 15175 MW; | 1BC099056776D5A0 CRC64; |
| SQ | SEQUENCE | 141 AA; | 15305 MW; | 86A8047BEF8A2171 CRC64; |
| SQ | SEQUENCE | 143 AA; | 15784 MW; | FFFBD93E07E0F09F CRC64; |
| SQ | SEQUENCE | 148 AA; | 16166 MW; | 68A987FB53A3BEB4 CRC64; |
| SQ | SEQUENCE | 141 AA; | 15250 MW; | C4F288661A1528B5 CRC64; |
| SQ | SEQUENCE | 142 AA; | 15867 MW; | 86ADC5E51EAFEB4E CRC64; |
| SQ | SEQUENCE | 143 AA; | 15638 MW; | 88570E5822D0D769 CRC64; |
| SQ | SEQUENCE | 143 AA; | 16092 MW; | 54E0C28213051123 CRC64; |
| SQ | SEQUENCE | 141 AA; | 15775 MW; | 14FBA19A3A81CD40 CRC64; |
| SQ | SEQUENCE | 141 AA; | 15010 MW; | 05E68C0CBA810D99 CRC64; |
| SQ | SEQUENCE | 141 AA; | 15219 MW; | 00A0F30D790C986F CRC64; |
| SQ | SEQUENCE | 141 AA; | 15248 MW; | 8DC8D74198A9E3F4 CRC64; |
| SQ | SEQUENCE | 141 AA; | 15437 MW; | D551EC4E85672284 CRC64; |
| SQ | SEQUENCE | 141 AA; | 14954 MW; | 0E4004454E776A25 CRC64; |
| SQ | SEQUENCE | 141 AA; | 15428 MW; | 8CE99D085937AC7B CRC64; |
| SQ | SEQUENCE | 141 AA; | 15125 MW; | 5B2EBB566F902D4F CRC64; |
| SQ | SEQUENCE | 141 AA; | 15334 MW; | 15CF4E62B18784FB CRC64; |
| SQ | SEQUENCE | 141 AA; | 14994 MW; | 66E874DFADD76CB5 CRC64; |
| SQ | SEQUENCE | 141 AA; | 15275 MW; | C4F1F3967F88F49B CRC64; |
| SQ | SEQUENCE | 141 AA; | 15369 MW; | 4ABCFF959AAB70AC CRC64; |
| SQ | SEQUENCE | 141 AA; | 15233 MW; | 5F870719E7CB166B CRC64; |
| SQ | SEQUENCE | 141 AA; | 15039 MW; | 95869BABB4C4EDB8 CRC64; |
| SQ | SEQUENCE | 142 AA; | 15576 MW; | 025D70E3EE3F068A CRC64; |
| SQ | SEQUENCE | 141 AA; | 15126 MW; | FB0EC4BBF2F01AF4 CRC64; |
| SQ | SEQUENCE | 141 AA; | 15457 MW; | F027DD88DD025544 CRC64; |
| SQ | SEQUENCE | 141 AA; | 15197 MW; | FFC6850B25D5A2F6 CRC64; |
| SQ | SEQUENCE | 141 AA; | 15409 MW; | 9DEDD577D589D333 CRC64; |
| SQ | SEQUENCE | 141 AA; | 15454 MW; | F72D8BDD2D25C501 CRC64; |
| SQ | SEQUENCE | 141 AA; | 15094 MW; | 2EA4BC1D75A6401E CRC64; |
| SQ | SEQUENCE | 141 AA; | 15033 MW; | E243F9624438B6F4 CRC64; |
| SQ | SEQUENCE | 141 AA; | 14982 MW; | 86C87D176F605941 CRC64; |
| SQ | SEQUENCE | 141 AA; | 15188 MW; | 242E1D9EE539DF31 CRC64; |
| SQ | SEQUENCE | 142 AA; | 15712 MW; | 7A7DF185D4A348FD CRC64; |
| SQ | SEQUENCE | 143 AA; | 15446 MW; | 994692213AB528F3 CRC64; |
| SQ | SEQUENCE | 141 AA; | 15647 MW; | 940FD2107C27F676 CRC64; |
| SQ | SEQUENCE | 141 AA; | 15229 MW; | 8D5B69010AE01AAF CRC64; |
| SQ | SEQUENCE | 141 AA; | 15437 MW; | 7DFD4C1661C01720 CRC64; |
| SQ | SEQUENCE | 141 AA; | 15756 MW; | 09969C7FEAD81A6F CRC64; |
| SQ | SEQUENCE | 146 AA; | 16281 MW; | 4CB775B6500CFE28 CRC64; |
| SQ | SEQUENCE | 146 AA; | 16364 MW; | 1DC991617574CF73 CRC64; |
| SQ | SEQUENCE | 146 AA; | 16158 MW; | 6DD7F7AE00A9077D CRC64; |
| SQ | SEQUENCE | 146 AA; | 16111 MW; | 097902EF83B05DA2 CRC64; |
| SQ | SEQUENCE | 146 AA; | 15709 MW; | C844408B2E2106A3 CRC64; |
| SQ | SEQUENCE | 146 AA; | 15881 MW; | 2065A57D9D1D6071 CRC64; |
| SQ | SEQUENCE | 146 AA; | 16203 MW; | 6CCA27543F32021C CRC64; |
| SQ | SEQUENCE | 146 AA; | 16191 MW; | E2714EE2794081DD CRC64; |
| SQ | SEQUENCE | 146 AA; | 15973 MW; | 1D53E28D108F6124 CRC64; |
| SQ | SEQUENCE | 142 AA; | 16131 MW; | 18976431996B8046 CRC64; |
| SQ | SEQUENCE | 145 AA; | 16003 MW; | BEF06F441B42BA80 CRC64; |
| SQ | SEQUENCE | 146 AA; | 16136 MW; | 3FFEE0435F245EE9 CRC64; |
| SQ | SEQUENCE | 146 AA; | 16193 MW; | 1D73BBFF3EAE04B7 CRC64; |
| SQ | SEQUENCE | 145 AA; | 16273 MW; | 0E7EBB0A76503D7F CRC64; |
| SQ | SEQUENCE | 146 AA; | 16352 MW; | 59A0FD9CF63B16B6 CRC64; |
| SQ | SEQUENCE | 146 AA; | 16330 MW; | 8513E50D47DDCD8D CRC64; |
| SQ | SEQUENCE | 146 AA; | 16290 MW; | C829E39F3AFD1B0D CRC64; |
| SQ | SEQUENCE | 146 AA; | 15855 MW; | AA82B6EEBE6466BD CRC64; |
| SQ | SEQUENCE | 146 AA; | 15851 MW; | D944CBC57EFF4BB8 CRC64; |
| SQ | SEQUENCE | 146 AA; | 16342 MW; | 1090F7074756ACBA CRC64; |
| SQ | SEQUENCE | 145 AA; | 16218 MW; | 04C8F25E427BAAC5 CRC64; |
| SQ | SEQUENCE | 145 AA; | 15988 MW; | 4F108FC787F397A7 CRC64; |
| SQ | SEQUENCE | 146 AA; | 15841 MW; | BB53347CA9BFFAEA CRC64; |
| SQ | SEQUENCE | 146 AA; | 15869 MW; | 61CC8AEA0AFC160E CRC64; |
| SQ | SEQUENCE | 146 AA; | 16346 MW; | 395C0DF6195E0810 CRC64; |
| SQ | SEQUENCE | 147 AA; | 15982 MW; | A4D7EA3004746476 CRC64; |
| SQ | SEQUENCE | 147 AA; | 16770 MW; | C447A8450208969F CRC64; |
| SQ | SEQUENCE | 145 AA; | 15964 MW; | 52685BDC8CDFBDD5 CRC64; |
| SQ | SEQUENCE | 147 AA; | 16241 MW; | 6A7A173B74D0EE89 CRC64; |
| SQ | SEQUENCE | 146 AA; | 15892 MW; | 3C9DF56756252C58 CRC64; |
| SQ | SEQUENCE | 141 AA; | 15620 MW; | 305CEA482FAC825C CRC64; |
| SQ | SEQUENCE | 146 AA; | 15976 MW; | 4D75EB9FC8D73539 CRC64; |
| SQ | SEQUENCE | 145 AA; | 15859 MW; | 78B8722915E9C221 CRC64; |
| SQ | SEQUENCE | 146 AA; | 15916 MW; | A1D03928EE41DAB9 CRC64; |
| SQ | SEQUENCE | 141 AA; | 15633 MW; | 394652AA5100FA33 CRC64; |
| SQ | SEQUENCE | 146 AA; | 16140 MW; | 532B435C899C41C2 CRC64; |
| SQ | SEQUENCE | 145 AA; | 16223 MW; | C2D22F363D3B78EA CRC64; |
| SQ | SEQUENCE | 146 AA; | 16610 MW; | 4A2E01EA768657A0 CRC64; |
| SQ | SEQUENCE | 146 AA; | 15898 MW; | 0B320D53704A60D6 CRC64; |
| SQ | SEQUENCE | 146 AA; | 16061 MW; | C0AEB956165213B0 CRC64; |
| SQ | SEQUENCE | 146 AA; | 16143 MW; | 233AB3CA9FDA83D5 CRC64; |
| SQ | SEQUENCE | 146 AA; | 16718 MW; | A224788EEDE940B4 CRC64; |
| SQ | SEQUENCE | 146 AA; | 16114 MW; | A25A35F5FB124AC2 CRC64; |
| SQ | SEQUENCE | 146 AA; | 15996 MW; | ECD68B81D53608F1 CRC64; |
| SQ | SEQUENCE | 147 AA; | 16210 MW; | 32F6EA73A1D52497 CRC64; |
| SQ | SEQUENCE | 146 AA; | 16602 MW; | CDED66122A208FDF CRC64; |
| SQ | SEQUENCE | 146 AA; | 15921 MW; | E880DAC410019685 CRC64; |
| SQ | SEQUENCE | 146 AA; | 16458 MW; | 7E7250E4B779C128 CRC64; |
| SQ | SEQUENCE | 146 AA; | 16304 MW; | 809F2AFC39F50FB3 CRC64; |
| SQ | SEQUENCE | 146 AA; | 16206 MW; | 23C009BE653EE623 CRC64; |
| SQ | SEQUENCE | 146 AA; | 16152 MW; | 3F9698269D2F06FD CRC64; |
| SQ | SEQUENCE | 146 AA; | 16017 MW; | FC1994B66F07CC34 CRC64; |
| SQ | SEQUENCE | 146 AA; | 16437 MW; | 0747C5B2E7E88BFB CRC64; |
| SQ | SEQUENCE | 146 AA; | 15846 MW; | F3D582E685E182F8 CRC64; |
| SQ | SEQUENCE | 146 AA; | 15927 MW; | 7BEC577E91F332AD CRC64; |
| SQ | SEQUENCE | 141 AA; | 16289 MW; | DAED4F578804D27B CRC64; |
| SQ | SEQUENCE | 146 AA; | 16326 MW; | D57A75D7F08F5405 CRC64; |
| SQ | SEQUENCE | 147 AA; | 16186 MW; | 447C639EC466D645 CRC64; |
| SQ | SEQUENCE | 146 AA; | 16079 MW; | 854211E2217E23AB CRC64; |
| SQ | SEQUENCE | 146 AA; | 16134 MW; | 43FFAED7AD7EBC9D CRC64; |
| SQ | SEQUENCE | 147 AA; | 15779 MW; | E171E03AD6916485 CRC64; |
| SQ | SEQUENCE | 146 AA; | 16168 MW; | 56F5DC4825A2D485 CRC64; |
| SQ | SEQUENCE | 146 AA; | 15805 MW; | 0E1520B80808DA2B CRC64; |
| SQ | SEQUENCE | 146 AA; | 16295 MW; | DDE87303B8B05342 CRC64; |
| SQ | SEQUENCE | 146 AA; | 15770 MW; | 20D8B50B7D6FCEFD CRC64; |
| SQ | SEQUENCE | 146 AA; | 16582 MW; | 71E26667C216001A CRC64; |
| SQ | SEQUENCE | 146 AA; | 15934 MW; | FC2BEB1E091FACEE CRC64; |
| SQ | SEQUENCE | 146 AA; | 16274 MW; | 3D754EEB0D242C28 CRC64; |
| SQ | SEQUENCE | 146 AA; | 16048 MW; | 321C61BBB206299B CRC64; |
| SQ | SEQUENCE | 141 AA; | 16011 MW; | 1744CBB71EBF402F CRC64; |
| SQ | SEQUENCE | 146 AA; | 16065 MW; | 70315747FBED6FE6 CRC64; |
| SQ | SEQUENCE | 146 AA; | 16008 MW; | 734664793DA642EE CRC64; |
| SQ | SEQUENCE | 146 AA; | 16871 MW; | E61EA7DBB67EFC52 CRC64; |
| SQ | SEQUENCE | 147 AA; | 16294 MW; | 253C91230838BE0C CRC64; |
| SQ | SEQUENCE | 146 AA; | 15981 MW; | EE1E981B35D2B151 CRC64; |
| SQ | SEQUENCE | 146 AA; | 15931 MW; | D9937E0F66281FDB CRC64; |
| SQ | SEQUENCE | 147 AA; | 16817 MW; | A9E490F00AE29324 CRC64; |
| SQ | SEQUENCE | 146 AA; | 15938 MW; | 6E7F7BBE515737E0 CRC64; |
| SQ | SEQUENCE | 146 AA; | 16006 MW; | F77CC19DCA08EE65 CRC64; |
| SQ | SEQUENCE | 146 AA; | 16015 MW; | 85DE1B116564AFCF CRC64; |
| SQ | SEQUENCE | 146 AA; | 15736 MW; | 7DD33D1F41EB2B97 CRC64; |
| SQ | SEQUENCE | 146 AA; | 16145 MW; | AD822D9385652470 CRC64; |
| SQ | SEQUENCE | 146 AA; | 15860 MW; | FA4C582505A52C92 CRC64; |
| SQ | SEQUENCE | 146 AA; | 16108 MW; | 00F652D17DC71DAF CRC64; |
| SQ | SEQUENCE | 146 AA; | 15672 MW; | 92FE8CB23CACE4C4 CRC64; |
| SQ | SEQUENCE | 137 AA; | 16074 MW; | 08F2165693645D98 CRC64; |
| SQ | SEQUENCE | 146 AA; | 15796 MW; | 6BED6EF2F929E840 CRC64; |
| SQ | SEQUENCE | 146 AA; | 16069 MW; | 28881A4B53F139F4 CRC64; |
| SQ | SEQUENCE | 145 AA; | 15824 MW; | F3875A54C4C84323 CRC64; |
| SQ | SEQUENCE | 146 AA; | 15723 MW; | 82136071CC4911F9 CRC64; |
| SQ | SEQUENCE | 146 AA; | 15872 MW; | E9043FEC82ADB2E1 CRC64; |
| SQ | SEQUENCE | 145 AA; | 16108 MW; | 50C7CDBB8AD3F5DD CRC64; |
| SQ | SEQUENCE | 146 AA; | 15986 MW; | FAB18B0F2C9486E5 CRC64; |
| SQ | SEQUENCE | 146 AA; | 16034 MW; | B542033A32FDDC93 CRC64; |
| SQ | SEQUENCE | 146 AA; | 15933 MW; | 69FFCC941EC360B8 CRC64; |
| SQ | SEQUENCE | 140 AA; | 15424 MW; | 01963DA6056020E5 CRC64; |
| SQ | SEQUENCE | 140 AA; | 15423 MW; | 151F75CF7076AAB0 CRC64; |
| SQ | SEQUENCE | 146 AA; | 15857 MW; | 09D6907DFE4EBF15 CRC64; |
| SQ | SEQUENCE | 147 AA; | 16141 MW; | 6217CFF78791DC6D CRC64; |
| SQ | SEQUENCE | 146 AA; | 15950 MW; | A60284DA068FEE86 CRC64; |
| SQ | SEQUENCE | 146 AA; | 15784 MW; | EDE5043E5275154A CRC64; |
| SQ | SEQUENCE | 142 AA; | 16140 MW; | EF83400A848A771A CRC64; |
| SQ | SEQUENCE | 146 AA; | 15734 MW; | D4914E46EB487432 CRC64; |
| SQ | SEQUENCE | 146 AA; | 15732 MW; | 90A42D3094129A09 CRC64; |
| SQ | SEQUENCE | 146 AA; | 15787 MW; | ADA727FE38EB53BC CRC64; |
| SQ | SEQUENCE | 146 AA; | 15862 MW; | 5B04784AF7F3C9D0 CRC64; |
| SQ | SEQUENCE | 146 AA; | 16181 MW; | 9BF40F0B599186DA CRC64; |
| SQ | SEQUENCE | 146 AA; | 16179 MW; | 4B895DC62239ACEB CRC64; |
| SQ | SEQUENCE | 146 AA; | 15855 MW; | EA329730E8832F73 CRC64; |
| SQ | SEQUENCE | 146 AA; | 15763 MW; | 03534396F8BADA21 CRC64; |
| SQ | SEQUENCE | 146 AA; | 15939 MW; | 9AD9691BFD0D0F24 CRC64; |
| SQ | SEQUENCE | 146 AA; | 16404 MW; | D807840F5AB1E090 CRC64; |
| SQ | SEQUENCE | 146 AA; | 16131 MW; | D13DD1BBC8407E30 CRC64; |
| SQ | SEQUENCE | 146 AA; | 16557 MW; | BEF4011A0BA96394 CRC64; |
| SQ | SEQUENCE | 146 AA; | 16472 MW; | 0444F500B91828E4 CRC64; |
| SQ | SEQUENCE | 146 AA; | 16130 MW; | 2A806D33CC34FB22 CRC64; |
| SQ | SEQUENCE | 146 AA; | 16005 MW; | F37CE4F46B377C88 CRC64; |
| SQ | SEQUENCE | 146 AA; | 15963 MW; | 71654BBD7D9F5A9E CRC64; |
| SQ | SEQUENCE | 146 AA; | 16159 MW; | 6F4192FE6DE56022 CRC64; |
| SQ | SEQUENCE | 146 AA; | 16061 MW; | 98C27F1216FFB801 CRC64; |
| SQ | SEQUENCE | 146 AA; | 16046 MW; | 045A1559E5A0D931 CRC64; |
| SQ | SEQUENCE | 141 AA; | 15062 MW; | 2868B507903E636A CRC64; |
| SQ | SEQUENCE | 146 AA; | 15823 MW; | C0480FA30551CA14 CRC64; |
| SQ | SEQUENCE | 146 AA; | 16014 MW; | 033A24B8E1C3802B CRC64; |
| SQ | SEQUENCE | 146 AA; | 16119 MW; | D585DFCEB2A15B07 CRC64; |
| SQ | SEQUENCE | 146 AA; | 16093 MW; | EB8D6C1C24DD2D82 CRC64; |
| SQ | SEQUENCE | 146 AA; | 16210 MW; | 09C3677E74AE0AB3 CRC64; |
| SQ | SEQUENCE | 158 AA; | 17871 MW; | 9E3E145432E0BEA0 CRC64; |
| SQ | SEQUENCE | 151 AA; | 17112 MW; | 570921E1FB8C205F CRC64; |
| SQ | SEQUENCE | 141 AA; | 15588 MW; | 8788FAFAB386D407 CRC64; |
| SQ | SEQUENCE | 153 AA; | 16622 MW; | F130A15E282F1F5D CRC64; |
| SQ | SEQUENCE | 147 AA; | 16055 MW; | DBD0A94E743CB28A CRC64; |
| SQ | SEQUENCE | 147 AA; | 15835 MW; | 6A23BB948C4EF4B9 CRC64; |
| SQ | SEQUENCE | 143 AA; | 15257 MW; | A166605B5A3013CD CRC64; |
| SQ | SEQUENCE | 143 AA; | 15399 MW; | B8F5582BD3D3DE33 CRC64; |
| SQ | SEQUENCE | 145 AA; | 15363 MW; | E900053FDF656A63 CRC64; |
| SQ | SEQUENCE | 153 AA; | 16652 MW; | FE29AB9DEF33AFC8 CRC64; |
| SQ | SEQUENCE | 146 AA; | 15752 MW; | 2013630DF6026743 CRC64; |
| SQ | SEQUENCE | 145 AA; | 15536 MW; | F954B275B0188842 CRC64; |
| SQ | SEQUENCE | 147 AA; | 15669 MW; | 94D372E3AE30B2DE CRC64; |
| SQ | SEQUENCE | 148 AA; | 16548 MW; | 6A74DA1E933BACBB CRC64; |
| SQ | SEQUENCE | 145 AA; | 15811 MW; | 1C3A1011EE398DB5 CRC64; |
| SQ | SEQUENCE | 145 AA; | 15744 MW; | A301D4B0FB22FC5C CRC64; |
| SQ | SEQUENCE | 147 AA; | 15929 MW; | 72F415B1D803273C CRC64; |
| SQ | SEQUENCE | 149 AA; | 16296 MW; | 073877B90BF81099 CRC64; |
| SQ | SEQUENCE | 147 AA; | 15755 MW; | E62DE1BE4E523E77 CRC64; |
| SQ | SEQUENCE | 145 AA; | 15551 MW; | A5A2B15657728759 CRC64; |
| SQ | SEQUENCE | 154 AA; | 17874 MW; | 782E2BC23CEC3C29 CRC64; |
| SQ | SEQUENCE | 153 AA; | 17030 MW; | E8EDB8D4616D020E CRC64; |
| SQ | SEQUENCE | 152 AA; | 17305 MW; | 90FFCCAFF54BBF9A CRC64; |
| SQ | SEQUENCE | 153 AA; | 17155 MW; | A5364E71B9705C6E CRC64; |
| SQ | SEQUENCE | 153 AA; | 16946 MW; | 4E69AC6BE2181728 CRC64; |
| SQ | SEQUENCE | 153 AA; | 17206 MW; | 94C7C1B43EB7B0F1 CRC64; |
| SQ | SEQUENCE | 153 AA; | 17391 MW; | 94475608DA8D75A0 CRC64; |
| SQ | SEQUENCE | 153 AA; | 17020 MW; | 4FD93C4E116B6D4D CRC64; |
| SQ | SEQUENCE | 153 AA; | 17291 MW; | 07AA57A1BA1EBC9C CRC64; |
| SQ | SEQUENCE | 146 AA; | 15596 MW; | F5B8864AEC251B77 CRC64; |
| SQ | SEQUENCE | 153 AA; | 16943 MW; | EB1676C5B7EBFB59 CRC64; |
| SQ | SEQUENCE | 146 AA; | 15645 MW; | 92083FF3EB31842C CRC64; |
| SQ | SEQUENCE | 153 AA; | 16995 MW; | 5823AB85F706EA9C CRC64; |
| SQ | SEQUENCE | 153 AA; | 16970 MW; | 0DB58E687ADC5733 CRC64; |
| SQ | SEQUENCE | 148 AA; | 16317 MW; | AC0C484FF4EED715 CRC64; |
| SQ | SEQUENCE | 153 AA; | 17297 MW; | E3458330A86DE9B4 CRC64; |
| SQ | SEQUENCE | 148 AA; | 16846 MW; | 586DAD8B1F7C7871 CRC64; |
| SQ | SEQUENCE | 153 AA; | 16951 MW; | 89CA01974231E93C CRC64; |
| SQ | SEQUENCE | 153 AA; | 17237 MW; | 5771A432C7B32614 CRC64; |
| SQ | SEQUENCE | 153 AA; | 17138 MW; | D757FEB0F8FC7542 CRC64; |
| SQ | SEQUENCE | 153 AA; | 16938 MW; | 27C153E2DA7E6AB9 CRC64; |
| SQ | SEQUENCE | 148 AA; | 16380 MW; | 3D74C2D0B5031C70 CRC64; |
| SQ | SEQUENCE | 153 AA; | 16966 MW; | C9C6113DA3FF5A6B CRC64; |
| SQ | SEQUENCE | 146 AA; | 15529 MW; | 74C72B21D005BD44 CRC64; |
| SQ | SEQUENCE | 153 AA; | 17435 MW; | D0CA8894E7E8B105 CRC64; |
| SQ | SEQUENCE | 153 AA; | 17249 MW; | 1A2C076D7FC27A3C CRC64; |
| SQ | SEQUENCE | 139 AA; | 15964 MW; | 454099E413848DA5 CRC64; |
| SQ | SEQUENCE | 143 AA; | 15431 MW; | 67A6BB463A3F4D49 CRC64; |
| SQ | SEQUENCE | 158 AA; | 17881 MW; | 48BB0C98E11F2513 CRC64; |
| SQ | SEQUENCE | 151 AA; | 16180 MW; | 4943B6B51A6D916E CRC64; |
| SQ | SEQUENCE | 147 AA; | 16540 MW; | 39BF594C21B3DC1E CRC64; |
| SQ | SEQUENCE | 147 AA; | 16162 MW; | D972A74BD717F717 CRC64; |
| SQ | SEQUENCE | 151 AA; | 17924 MW; | FBA51F54A245FBD9 CRC64; |
| SQ | SEQUENCE | 140 AA; | 16080 MW; | B1B509FBDFF9BAF5 CRC64; |
| SQ | SEQUENCE | 141 AA; | 15551 MW; | B1029CBF59A7108F CRC64; |
| SQ | SEQUENCE | 154 AA; | 17094 MW; | 62D74615287A10EC CRC64; |
| SQ | SEQUENCE | 151 AA; | 16337 MW; | 6026DA2CD632C7C8 CRC64; |
| SQ | SEQUENCE | 151 AA; | 15986 MW; | DDCA9BEB2B923C35 CRC64; |
| SQ | SEQUENCE | 155 AA; | 17642 MW; | 64FB97585E52E6B5 CRC64; |
| SQ | SEQUENCE | 152 AA; | 17331 MW; | E3B6E0ADB9CD2F00 CRC64; |
| SQ | SEQUENCE | 158 AA; | 17625 MW; | 6105EB7611A7A079 CRC64; |
| SQ | SEQUENCE | 145 AA; | 16825 MW; | 02104CB584134DF4 CRC64; |
| SQ | SEQUENCE | 151 AA; | 16265 MW; | D41360F7A9AE2863 CRC64; |
| SQ | SEQUENCE | 148 AA; | 15900 MW; | 53CFAB27366118C3 CRC64; |
| SQ | SEQUENCE | 147 AA; | 16022 MW; | 4A11053B63C53F50 CRC64; |
| SQ | SEQUENCE | 142 AA; | 15525 MW; | 92B3CA50D9CD8619 CRC64; |
| SQ | SEQUENCE | 144 AA; | 15757 MW; | 5112E67DC4A602B2 CRC64; |
| SQ | SEQUENCE | 147 AA; | 16375 MW; | 742905A529BDFB22 CRC64; |
| SQ | SEQUENCE | 147 AA; | 16706 MW; | 7A89DE6847D49DD6 CRC64; |
| SQ | SEQUENCE | 143 AA; | 15799 MW; | 4FB5A252D76534D4 CRC64; |
| SQ | SEQUENCE | 151 AA; | 16997 MW; | 3F2BD77522B9CA3D CRC64; |
| SQ | SEQUENCE | 153 AA; | 17302 MW; | 44FE83C4F451C006 CRC64; |
| SQ | SEQUENCE | 138 AA; | 15793 MW; | 6510E13A6CA30DE8 CRC64; |
| SQ | SEQUENCE | 146 AA; | 16960 MW; | 912BA10B3AE4FD2E CRC64; |
| SQ | SEQUENCE | 158 AA; | 18585 MW; | 5E2BC405C835C3C7 CRC64; |
| SQ | SEQUENCE | 147 AA; | 16365 MW; | C933A2810304AD9C CRC64; |
| SQ | SEQUENCE | 159 AA; | 17761 MW; | 1C9386169E13484B CRC64; |
| SQ | SEQUENCE | 142 AA; | 15111 MW; | 2DB1EF4602F929D3 CRC64; |
| SQ | SEQUENCE | 159 AA; | 17684 MW; | 6408F865A66D6D37 CRC64; |
| SQ | SEQUENCE | 142 AA; | 15326 MW; | 6CB0C6462103CB6F CRC64; |
| SQ | SEQUENCE | 147 AA; | 16544 MW; | EC389C1010ECDFEE CRC64; |
| SQ | SEQUENCE | 151 AA; | 15734 MW; | EDCA6856DE7DDB12 CRC64; |
| SQ | SEQUENCE | 159 AA; | 18097 MW; | 9DB8A6F96C67820D CRC64; |
| SQ | SEQUENCE | 156 AA; | 17755 MW; | CCF32AC51F1CC114 CRC64; |
| SQ | SEQUENCE | 152 AA; | 17197 MW; | 35F7A94D887B89AA CRC64; |
| SQ | SEQUENCE | 147 AA; | 16350 MW; | 47510F7C2D569B74 CRC64; |
| SQ | SEQUENCE | 147 AA; | 16108 MW; | 54AD783F0B5BF488 CRC64; |
| SQ | SEQUENCE | 159 AA; | 17703 MW; | 0B323149898B1FAF CRC64; |
| SQ | SEQUENCE | 146 AA; | 15396 MW; | 307DAA2C6D9FDC27 CRC64; |
| SQ | SEQUENCE | 147 AA; | 15766 MW; | 44EB9A4611EE0366 CRC64; |
| SQ | SEQUENCE | 147 AA; | 15797 MW; | D0864510EE730506 CRC64; |
| SQ | SEQUENCE | 147 AA; | 15842 MW; | F52D010973F4D84B CRC64; |
| SQ | SEQUENCE | 152 AA; | 16533 MW; | 094FC632B8523E73 CRC64; |
| SQ | SEQUENCE | 147 AA; | 17369 MW; | 4362D4DD89360A30 CRC64; |
| SQ | SEQUENCE | 140 AA; | 16082 MW; | 93628095A7C366EC CRC64; |
| SQ | SEQUENCE | 146 AA; | 16324 MW; | 485908091488B8CC CRC64; |
| SQ | SEQUENCE | 147 AA; | 16006 MW; | A96F1D05A4AD4727 CRC64; |
| SQ | SEQUENCE | 142 AA; | 15491 MW; | 18D7FE0DE7565D6E CRC64; |
| SQ | SEQUENCE | 144 AA; | 15623 MW; | CE4BDEFAC7D4C22C CRC64; |
| SQ | SEQUENCE | 153 AA; | 17102 MW; | 383D7685F3BEE707 CRC64; |
| SQ | SEQUENCE | 149 AA; | 17173 MW; | 4E463357F89FDA64 CRC64; |
| SQ | SEQUENCE | 144 AA; | 16092 MW; | 312E087C1B112D8E CRC64; |
| SQ | SEQUENCE | 156 AA; | 17779 MW; | 3FE4830DE3A18CD7 CRC64; |
| SQ | SEQUENCE | 149 AA; | 16915 MW; | 36B98205BB87C7D4 CRC64; |
| SQ | SEQUENCE | 147 AA; | 16655 MW; | D7CBCC5F971762E8 CRC64; |
| SQ | SEQUENCE | 147 AA; | 16396 MW; | 79E874B4EFAA13EA CRC64; |
| SQ | SEQUENCE | 143 AA; | 15613 MW; | 34EB2B649AF9E328 CRC64; |
| SQ | SEQUENCE | 143 AA; | 15654 MW; | 5E6B2917777D0CB2 CRC64; |

TABLE 8
Header information for 55 lysozyme sequences used in the fold recognition
analysis. Sequences obtained from Swiss-Prot database.

| | | | | |
|---|---|---|---|---|
| SQ | SEQUENCE | 147 AA; | 16363 MW; | B0F2B6A9F7DA3978 CRC64; |
| SQ | SEQUENCE | 129 AA; | 14471 MW; | 64CD8C3F9C80359A CRC64; |
| SQ | SEQUENCE | 129 AA; | 14433 MW; | DA7FD76F890AFE91 CRC64; |
| SQ | SEQUENCE | 129 AA; | 14652 MW; | 87040531F4B60F46 CRC64; |
| SQ | SEQUENCE | 128 AA; | 14668 MW; | FD169D39228DF774 CRC64; |
| SQ | SEQUENCE | 148 AA; | 16729 MW; | 8E6E986539BD3EEE CRC64; |
| SQ | SEQUENCE | 125 AA; | 13994 MW; | 18D8A1DBA3724073 CRC64; |
| SQ | SEQUENCE | 130 AA; | 14578 MW; | 96C9BA30478D60F6 CRC64; |
| SQ | SEQUENCE | 144 AA; | 15737 MW; | BF945ADCDCC2D668 CRC64; |
| SQ | SEQUENCE | 148 AA; | 16618 MW; | 2855279E91CCC083 CRC64; |
| SQ | SEQUENCE | 130 AA; | 14611 MW; | C79D70D3B8F70A8E CRC64; |
| SQ | SEQUENCE | 148 AA; | 16689 MW; | 5C768DDCD8071BAF CRC64; |
| SQ | SEQUENCE | 177 AA; | 19607 MW; | E546FDCB1201F036 CRC64; |
| SQ | SEQUENCE | 146 AA; | 16330 MW; | AF9689BC02218086 CRC64; |
| SQ | SEQUENCE | 171 AA; | 18884 MW; | 9F53C6ADF47D4789 CRC64; |
| SQ | SEQUENCE | 178 AA; | 19597 MW; | D447BEAB4E0B2CDA CRC64; |
| SQ | SEQUENCE | 165 AA; | 17996 MW; | 14ECECD883232D3C CRC64; |
| SQ | SEQUENCE | 146 AA; | 16138 MW; | 3A16675AB1A635EB CRC64; |
| SQ | SEQUENCE | 167 AA; | 18232 MW; | E1D72ACCA49E32FC CRC64; |
| SQ | SEQUENCE | 177 AA; | 19662 MW; | 52DCCA3D315AC888 CRC64; |
| SQ | SEQUENCE | 140 AA; | 15398 MW; | 93AD614699216C28 CRC64; |
| SQ | SEQUENCE | 129 AA; | 14446 MW; | A0AA48E69C2EB383 CRC64; |
| SQ | SEQUENCE | 137 AA; | 15668 MW; | FFE5710506C61A1D CRC64; |
| SQ | SEQUENCE | 148 AA; | 16602 MW; | C79BB37557B1E951 CRC64; |
| SQ | SEQUENCE | 130 AA; | 14795 MW; | 6CFFCB41B02FD287 CRC64; |
| SQ | SEQUENCE | 147 AA; | 16238 MW; | 81E85743FF579468 CRC64; |
| SQ | SEQUENCE | 148 AA; | 16258 MW; | 2402FC175CA271AA CRC64; |
| SQ | SEQUENCE | 127 AA; | 14452 MW; | 96D3BAFDFD934DD8 CRC64; |
| SQ | SEQUENCE | 121 AA; | 14027 MW; | 14E19F67523D263C CRC64; |
| SQ | SEQUENCE | 148 AA; | 16567 MW; | A3CFD26983BA6A9D CRC64; |
| SQ | SEQUENCE | 139 AA; | 15877 MW; | E8AF5C2CE561F641 CRC64; |
| SQ | SEQUENCE | 142 AA; | 16240 MW; | 7A565C7748D4C127 CRC64; |
| SQ | SEQUENCE | 145 AA; | 16268 MW; | FCDA921A2CAE7E94 CRC64; |
| SQ | SEQUENCE | 129 AA; | 14509 MW; | 079C91A8C604E218 CRC64; |
| SQ | SEQUENCE | 130 AA; | 14722 MW; | 000A3330EDB26C25 CRC64; |
| SQ | SEQUENCE | 141 AA; | 16271 MW; | 64510118422161E8 CRC64; |
| SQ | SEQUENCE | 147 AA; | 16839 MW; | F281667A17F483AC CRC64; |
| SQ | SEQUENCE | 140 AA; | 15635 MW; | 75C24CA6F85DF903 CRC64; |
| SQ | SEQUENCE | 141 AA; | 15648 MW; | 4D7C51B018FD5417 CRC64; |
| SQ | SEQUENCE | 140 AA; | 15651 MW; | ACD139CC656EF8FC CRC64; |
| SQ | SEQUENCE | 142 AA; | 15591 MW; | 2A48035364B995BC CRC64; |
| SQ | SEQUENCE | 143 AA; | 15611 MW; | 81FECBD9C2F298D5 CRC64; |
| SQ | SEQUENCE | 148 AA; | 16956 MW; | C79DEC3B68ADF117 CRC64; |
| SQ | SEQUENCE | 141 AA; | 16016 MW; | E6974FE84417FFCF CRC64; |
| SQ | SEQUENCE | 138 AA; | 16087 MW; | F291EB7A36BDB9C8 CRC64; |
| SQ | SEQUENCE | 147 AA; | 15929 MW; | 659AD5E5167BDDDD CRC64; |
| SQ | SEQUENCE | 153 AA; | 16817 MW; | 4D0DF93F4443C293 CRC64; |
| SQ | SEQUENCE | 158 AA; | 18092 MW; | 7DE82449862BF66B CRC64; |
| SQ | SEQUENCE | 146 AA; | 16227 MW; | 619EA94ED446282C CRC64; |
| SQ | SEQUENCE | 148 AA; | 16656 MW; | 4674158A2A5912F2 CRC64; |
| SQ | SEQUENCE | 158 AA; | 17208 MW; | 3538DBE1C825CDD3 CRC64; |
| SQ | SEQUENCE | 143 AA; | 16169 MW; | 6335792BE5E6C736 CRC64; |
| SQ | SEQUENCE | 148 AA; | 16965 MW; | 8C07C675470F1FFD CRC64; |
| SQ | SEQUENCE | 145 AA; | 16625 MW; | 1A83FDDF66B331E6 CRC64; |
| SQ | SEQUENCE | 143 AA; | 15774 MW; | 65F353FF5778988F CRC64; |

TABLE 9
Header information for 159 thioredoxin sequences used in the fold recognition
analysis. Sequences obtained from Swiss-Prot database.

| | | | | |
|---|---|---|---|---|
| SQ | SEQUENCE | 114 AA; | 12673 MW; | E090761B2187F1F6 CRC64; |
| SQ | SEQUENCE | 106 AA; | 11589 MW; | 78AA2B23312D7C3A CRC64; |
| SQ | SEQUENCE | 101 AA; | 11247 MW; | BA78E5511900B754 CRC64; |
| SQ | SEQUENCE | 105 AA; | 11279 MW; | 028E8E31FE19FC31 CRC64; |
| SQ | SEQUENCE | 105 AA; | 11926 MW; | 0BC12C2867CEB1F5 CRC64; |
| SQ | SEQUENCE | 107 AA; | 12384 MW; | 6A716FD55690C53B CRC64; |
| SQ | SEQUENCE | 106 AA; | 11517 MW; | 7C8F078C3AD9BAF4 CRC64; |
| SQ | SEQUENCE | 105 AA; | 11802 MW; | 19958B167EFAAC13 CRC64; |
| SQ | SEQUENCE | 110 AA; | 12235 MW; | 0CFBD6812E86C888 CRC64; |
| SQ | SEQUENCE | 108 AA; | 11727 MW; | 03F2478DE518B530 CRC64; |
| SQ | SEQUENCE | 107 AA; | 11585 MW; | C21EB09648FAFA1C CRC64; |
| SQ | SEQUENCE | 114 AA; | 12696 MW; | D67E87151114C4CE CRC64; |
| SQ | SEQUENCE | 109 AA; | 12171 MW; | C2AFC18F72A35BBC CRC64; |
| SQ | SEQUENCE | 104 AA; | 11754 MW; | 012189232A888134 CRC64; |
| SQ | SEQUENCE | 105 AA; | 11576 MW; | E03F636DFB3C3745 CRC64; |
| SQ | SEQUENCE | 103 AA; | 11262 MW; | 276B5E5DF5B98F2D CRC64; |
| SQ | SEQUENCE | 104 AA; | 11681 MW; | 506CFF9696A2208D CRC64; |
| SQ | SEQUENCE | 108 AA; | 12347 MW; | 703632A814DFA257 CRC64; |
| SQ | SEQUENCE | 108 AA; | 12439 MW; | F3204CE0A323E9AE CRC64; |
| SQ | SEQUENCE | 109 AA; | 12396 MW; | 64D333E010A67DD0 CRC64; |
| SQ | SEQUENCE | 104 AA; | 11569 MW; | 60B66B759010BB12 CRC64; |
| SQ | SEQUENCE | 102 AA; | 11159 MW; | F1B57486973A6ED4 CRC64; |
| SQ | SEQUENCE | 108 AA; | 11776 MW; | 13FB9A15325AE48E CRC64; |
| SQ | SEQUENCE | 102 AA; | 11147 MW; | C171B646D393428C CRC64; |
| SQ | SEQUENCE | 102 AA; | 11292 MW; | 76A190218324BA68 CRC64; |
| SQ | SEQUENCE | 107 AA; | 11727 MW; | 16DC91C849015D9D CRC64; |
| SQ | SEQUENCE | 107 AA; | 11874 MW; | 7F1117FFDBF3FAF9 CRC64; |
| SQ | SEQUENCE | 106 AA; | 11772 MW; | 05A2155B210E8C69 CRC64; |
| SQ | SEQUENCE | 107 AA; | 12032 MW; | 2ACD86CED51269E5 CRC64; |
| SQ | SEQUENCE | 102 AA; | 11657 MW; | 84365185B46F7D1D CRC64; |
| SQ | SEQUENCE | 107 AA; | 11556 MW; | BE9525588D1E7EA2 CRC64; |
| SQ | SEQUENCE | 108 AA; | 11675 MW; | C982277843F37D26 CRC64; |
| SQ | SEQUENCE | 109 AA; | 11564 MW; | D6E76BBB20C82460 CRC64; |
| SQ | SEQUENCE | 110 AA; | 12142 MW; | E8B74CF8CAF19414 CRC64; |
| SQ | SEQUENCE | 106 AA; | 11992 MW; | A7D1C5D2D44DBA19 CRC64; |
| SQ | SEQUENCE | 109 AA; | 12210 MW; | 8B42FEDA431F47D3 CRC64; |
| SQ | SEQUENCE | 107 AA; | 11681 MW; | 1725C8A244685477 CRC64; |
| SQ | SEQUENCE | 106 AA; | 11855 MW; | 0616F0DC47695967 CRC64; |
| SQ | SEQUENCE | 107 AA; | 11952 MW; | 01DD342F138CF75F CRC64; |
| SQ | SEQUENCE | 103 AA; | 11620 MW; | 01F6A77434559A46 CRC64; |
| SQ | SEQUENCE | 104 AA; | 11544 MW; | 60BE6196090AC773 CRC64; |
| SQ | SEQUENCE | 102 AA; | 11498 MW; | FC08F02C4170EA2D CRC64; |
| SQ | SEQUENCE | 102 AA; | 11215 MW; | 0D17B97E976FC144 CRC64; |
| SQ | SEQUENCE | 109 AA; | 12786 MW; | 7CBB63470D060F46 CRC64; |
| SQ | SEQUENCE | 112 AA; | 11837 MW; | 48F4ABF1CEE6D746 CRC64; |
| SQ | SEQUENCE | 104 AA; | 11872 MW; | 852B96C8EF850AFB CRC64; |
| SQ | SEQUENCE | 106 AA; | 11476 MW; | 6F057D9AA5FA4582 CRC64; |
| SQ | SEQUENCE | 106 AA; | 11265 MW; | 43FE12BFAA2DA786 CRC64; |
| SQ | SEQUENCE | 107 AA; | 11752 MW; | 038D5FDE3765EE58 CRC64; |
| SQ | SEQUENCE | 108 AA; | 11870 MW; | 908A6E87385C6AD8 CRC64; |
| SQ | SEQUENCE | 104 AA; | 11629 MW; | C4B66EE5EBEC231F CRC64; |
| SQ | SEQUENCE | 104 AA; | 11314 MW; | 4A31E015FD71AE03 CRC64; |
| SQ | SEQUENCE | 105 AA; | 11212 MW; | 78BA3F6721071C31 CRC64; |
| SQ | SEQUENCE | 105 AA; | 11971 MW; | 7D0391B157EE94E8 CRC64; |
| SQ | SEQUENCE | 102 AA; | 11166 MW; | 7069F4ACDAC34595 CRC64; |
| SQ | SEQUENCE | 107 AA; | 11483 MW; | 9AB62438E065EFAF CRC64; |
| SQ | SEQUENCE | 110 AA; | 11891 MW; | 18AA0F89F7E513A8 CRC64; |
| SQ | SEQUENCE | 106 AA; | 11617 MW; | 04ADC09C1901FFC0 CRC64; |
| SQ | SEQUENCE | 108 AA; | 11979 MW; | 76200C2FF2AD067F CRC64; |
| SQ | SEQUENCE | 105 AA; | 11391 MW; | 637ABB85D9AFE867 CRC64; |
| SQ | SEQUENCE | 103 AA; | 11073 MW; | 92E6CC4ADF057D31 CRC64; |
| SQ | SEQUENCE | 102 AA; | 11104 MW; | 446EA348281B6C0C CRC64; |
| SQ | SEQUENCE | 101 AA; | 11476 MW; | 7678E87CFB82B098 CRC64; |
| SQ | SEQUENCE | 104 AA; | 11745 MW; | 29809607F6CEB9C4 CRC64; |
| SQ | SEQUENCE | 105 AA; | 11963 MW; | 2E994D2E4FB77B4B CRC64; |
| SQ | SEQUENCE | 107 AA; | 12747 MW; | 0072722403F70871 CRC64; |
| SQ | SEQUENCE | 106 AA; | 12432 MW; | FB9C15D875A0FCC9 CRC64; |
| SQ | SEQUENCE | 104 AA; | 11825 MW; | C133AFEBF027A001 CRC64; |
| SQ | SEQUENCE | 104 AA; | 12472 MW; | 08FD42B356A66946 CRC64; |
| SQ | SEQUENCE | 104 AA; | 11478 MW; | B3689CCCC245EE87 CRC64; |
| SQ | SEQUENCE | 110 AA; | 12838 MW; | 048FD2E895B35260 CRC64; |
| SQ | SEQUENCE | 104 AA; | 11502 MW; | 8BDDD7A9F0327638 CRC64; |
| SQ | SEQUENCE | 108 AA; | 12068 MW; | 4B0A42ADE0623623 CRC64; |
| SQ | SEQUENCE | 104 AA; | 11962 MW; | 2B679B07144FF896 CRC64; |
| SQ | SEQUENCE | 104 AA; | 11594 MW; | AA332E989336D0D0 CRC64; |
| SQ | SEQUENCE | 112 AA; | 12613 MW; | BAECDB8204BCDE0C CRC64; |
| SQ | SEQUENCE | 109 AA; | 12030 MW; | D3BFA816794F3199 CRC64; |
| SQ | SEQUENCE | 110 AA; | 12569 MW; | AE4CC4DF17D80337 CRC64; |
| SQ | SEQUENCE | 113 AA; | 12557 MW; | 880236A06F78C3CF CRC64; |
| SQ | SEQUENCE | 108 AA; | 11715 MW; | 4A2438748744A6B1 CRC64; |
| SQ | SEQUENCE | 109 AA; | 11718 MW; | 4CE51A57E2CAB88F CRC64; |
| SQ | SEQUENCE | 109 AA; | 11953 MW; | 82C4B67C95432B0D CRC64; |
| SQ | SEQUENCE | 106 AA; | 11904 MW; | 4E708967FAD7C7C6 CRC64; |
| SQ | SEQUENCE | 106 AA; | 12284 MW; | 1E55F68CD0462EA6 CRC64; |
| SQ | SEQUENCE | 103 AA; | 11296 MW; | D5249AFC673378BF CRC64; |
| SQ | SEQUENCE | 110 AA; | 12207 MW; | 87B38417CDCF501A CRC64; |
| SQ | SEQUENCE | 105 AA; | 11820 MW; | A6714BA4018E04DA CRC64; |
| SQ | SEQUENCE | 106 AA; | 11299 MW; | 02289008B464B239 CRC64; |
| SQ | SEQUENCE | 104 AA; | 11412 MW; | 4D8B10D9F417FDA5 CRC64; |
| SQ | SEQUENCE | 107 AA; | 12675 MW; | 6573F3A2CD510676 CRC64; |
| SQ | SEQUENCE | 103 AA; | 12007 MW; | 3F68048809930A1E CRC64; |
| SQ | SEQUENCE | 104 AA; | 11443 MW; | DBBCE61D2DA7E770 CRC64; |
| SQ | SEQUENCE | 107 AA; | 12402 MW; | 721DC33004B1FB2C CRC64; |
| SQ | SEQUENCE | 106 AA; | 12330 MW; | 7FCF75342CCD8786 CRC64; |
| SQ | SEQUENCE | 107 AA; | 11671 MW; | FB0CBFDA7E6103D4 CRC64; |
| SQ | SEQUENCE | 104 AA; | 11477 MW; | F1881EF70FE93AE2 CRC64; |
| SQ | SEQUENCE | 109 AA; | 12768 MW; | 9F24DAEE51347BFB CRC64; |
| SQ | SEQUENCE | 104 AA; | 11446 MW; | 2181E719B9756456 CRC64; |
| SQ | SEQUENCE | 107 AA; | 12573 MW; | 9F5A1C925CB0245D CRC64; |
| SQ | SEQUENCE | 108 AA; | 11889 MW; | 01983FCB9EAF6D7D CRC64; |
| SQ | SEQUENCE | 105 AA; | 12461 MW; | F80566227F4F32C3 CRC64; |
| SQ | SEQUENCE | 104 AA; | 11588 MW; | 442D99FFEE7DCC98 CRC64; |
| SQ | SEQUENCE | 106 AA; | 12342 MW; | 467EE1C4DC898AD8 CRC64; |
| SQ | SEQUENCE | 113 AA; | 13187 MW; | 5E17549DB855AD72 CRC64; |
| SQ | SEQUENCE | 106 AA; | 11741 MW; | ED26DD622274321A CRC64; |
| SQ | SEQUENCE | 107 AA; | 11797 MW; | F5418621302FB8C9 CRC64; |
| SQ | SEQUENCE | 113 AA; | 12578 MW; | B8B1EFD101A1F07B CRC64; |
| SQ | SEQUENCE | 112 AA; | 12265 MW; | 4BA75628143470EB CRC64; |
| SQ | SEQUENCE | 105 AA; | 12270 MW; | 2E2EF49771CFD60A CRC64; |
| SQ | SEQUENCE | 106 AA; | 11148 MW; | 4858114FC058969A CRC64; |
| SQ | SEQUENCE | 110 AA; | 12689 MW; | F66351D927C67B6A CRC64; |
| SQ | SEQUENCE | 113 AA; | 12172 MW; | C92CC93F2CC7908E CRC64; |
| SQ | SEQUENCE | 109 AA; | 12096 MW; | 23D5A15376013707 CRC64; |
| SQ | SEQUENCE | 105 AA; | 12315 MW; | AAE653487B6B9DA0 CRC64; |
| SQ | SEQUENCE | 103 AA; | 11342 MW; | 27E003A221A4C780 CRC64; |
| SQ | SEQUENCE | 108 AA; | 11988 MW; | 18660F3EB46ED144 CRC64; |
| SQ | SEQUENCE | 104 AA; | 11524 MW; | AB17B08A76D4B42B CRC64; |
| SQ | SEQUENCE | 114 AA; | 12576 MW; | 20E9D103789BDDBF CRC64; |
| SQ | SEQUENCE | 106 AA; | 11924 MW; | CBB0C674ABE2F22F CRC64; |
| SQ | SEQUENCE | 102 AA; | 11386 MW; | CE088E0C29F50175 CRC64; |
| SQ | SEQUENCE | 104 AA; | 11611 MW; | EBF6209661939300 CRC64; |
| SQ | SEQUENCE | 105 AA; | 11943 MW; | D82B5FFB8FE037C0 CRC64; |
| SQ | SEQUENCE | 108 AA; | 11712 MW; | 4C3BA0E4836AEA15 CRC64; |
| SQ | SEQUENCE | 108 AA; | 12098 MW; | 3202D6CA506AF8D0 CRC64; |
| SQ | SEQUENCE | 113 AA; | 12809 MW; | 9A9D5CFD295072E7 CRC64; |
| SQ | SEQUENCE | 105 AA; | 12083 MW; | E16005293EEC9BF2 CRC64; |
| SQ | SEQUENCE | 110 AA; | 11991 MW; | F565C172BE3C7281 CRC64; |
| SQ | SEQUENCE | 108 AA; | 11795 MW; | 0BFA240678724F55 CRC64; |
| SQ | SEQUENCE | 107 AA; | 11390 MW; | B2CB667AA9ADC6CC CRC64; |
| SQ | SEQUENCE | 111 AA; | 12555 MW; | D41485BEDEC4B58F CRC64; |
| SQ | SEQUENCE | 104 AA; | 11431 MW; | 07D453D14FC68A41 CRC64; |
| SQ | SEQUENCE | 106 AA; | 12022 MW; | F4F276071B5C7B09 CRC64; |
| SQ | SEQUENCE | 110 AA; | 12768 MW; | 5E9FA3A0AF3219A8 CRC64; |
| SQ | SEQUENCE | 105 AA; | 11568 MW; | 94EB4AFDD6FF6BCF CRC64; |
| SQ | SEQUENCE | 105 AA; | 12024 MW; | ACDFF50CFA6CDAB8 CRC64; |
| SQ | SEQUENCE | 105 AA; | 12249 MW; | 04BE896B669C4CCE CRC64; |
| SQ | SEQUENCE | 106 AA; | 11431 MW; | CD871CA040EBD453 CRC64; |
| SQ | SEQUENCE | 107 AA; | 11449 MW; | 5E17A372E55896C1 CRC64; |
| SQ | SEQUENCE | 103 AA; | 11855 MW; | 2D27267531673263 CRC64; |
| SQ | SEQUENCE | 107 AA; | 11595 MW; | 7D7D61EEACD29781 CRC64; |
| SQ | SEQUENCE | 107 AA; | 11676 MW; | CF4E6EAF85BE3776 CRC64; |
| SQ | SEQUENCE | 105 AA; | 12043 MW; | FFF67F75B0FDF058 CRC64; |
| SQ | SEQUENCE | 105 AA; | 11972 MW; | 19DA0A9508FE5727 CRC64; |
| SQ | SEQUENCE | 113 AA; | 13227 MW; | 43BBFE364DE5A700 CRC64; |
| SQ | SEQUENCE | 108 AA; | 11933 MW; | D52AE9BA2D259AAD CRC64; |
| SQ | SEQUENCE | 110 AA; | 11817 MW; | DACAB212199FDE44 CRC64; |
| SQ | SEQUENCE | 106 AA; | 12872 MW; | 7CE8913AB92D90FB CRC64; |
| SQ | SEQUENCE | 104 AA; | 11437 MW; | CCC3F60A75B40989 CRC64; |
| SQ | SEQUENCE | 104 AA; | 11656 MW; | 7EB1027954D7045C CRC64; |
| SQ | SEQUENCE | 104 AA; | 11482 MW; | E1D1D0883B417445 CRC64; |
| SQ | SEQUENCE | 104 AA; | 11716 MW; | 0129998AEB88770C CRC64; |
| SQ | SEQUENCE | 107 AA; | 12135 MW; | 59A54EF9B2AA2B3C CRC64; |
| SQ | SEQUENCE | 107 AA; | 12085 MW; | 8B8E404B18F9A0E7 CRC64; |
| SQ | SEQUENCE | 104 AA; | 11353 MW; | AFF3AA62656E1151 CRC64; |
| SQ | SEQUENCE | 106 AA; | 12216 MW; | 3EE681D8DEE3E32F CRC64; |
| SQ | SEQUENCE | 104 AA; | 11683 MW; | 09B35CB9D01D4C55 CRC64; |
| SQ | SEQUENCE | 106 AA; | 11753 MW; | B668D234B6664A14 CRC64; |
| SQ | SEQUENCE | 106 AA; | 11851 MW; | 952F01A04686985F CRC64; |
| SQ | SEQUENCE | 104 AA; | 12387 MW; | BCFAA36EFD10876B CRC64; |

TABLE 10
Header information for 47 trypsin sequences used in the fold recognition analysis.
Sequences obtained from Swiss-Prot database.

| | | | | | | | |
|---|---|---|---|---|---|---|---|
| SQ | SEQUENCE | 218 | AA; | 23677 | MW; | 509AB50DE190EB39 | CRC64; |
| SQ | SEQUENCE | 245 | AA; | 25666 | MW; | 91A9F28E2F3E3142 | CRC64; |
| SQ | SEQUENCE | 245 | AA; | 26260 | MW; | 74FE0D425517AB02 | CRC64; |
| SQ | SEQUENCE | 243 | AA; | 25425 | MW; | B155CD91B89F61B8 | CRC64; |
| SQ | SEQUENCE | 248 | AA; | 26069 | MW; | C4CF589912B23D98 | CRC64; |
| SQ | SEQUENCE | 241 | AA; | 25941 | MW; | 44EC9A0106AD1A68 | CRC64; |
| SQ | SEQUENCE | 247 | AA; | 26558 | MW; | DD49A487B8062813 | CRC64; |
| SQ | SEQUENCE | 246 | AA; | 25959 | MW; | 6AFA0DAD11943FB5 | CRC64; |
| SQ | SEQUENCE | 242 | AA; | 25958 | MW; | 43F5642498067E5A | CRC64; |
| SQ | SEQUENCE | 243 | AA; | 25492 | MW; | C5B8345A8B3F8031 | CRC64; |
| SQ | SEQUENCE | 247 | AA; | 26289 | MW; | 50A070495A7731DB | CRC64; |
| SQ | SEQUENCE | 247 | AA; | 26423 | MW; | 374E9D31D6DB8EAF | CRC64; |
| SQ | SEQUENCE | 247 | AA; | 26488 | MW; | 82B0F41EB8E3D5DB | CRC64; |
| SQ | SEQUENCE | 246 | AA; | 26228 | MW; | A8D3630809AEE606 | CRC64; |
| SQ | SEQUENCE | 244 | AA; | 26079 | MW; | C63F29CB3300B323 | CRC64; |
| SQ | SEQUENCE | 248 | AA; | 26622 | MW; | E5E16B07622B588E | CRC64; |
| SQ | SEQUENCE | 247 | AA; | 26269 | MW; | D74892BAA584E4A8 | CRC64; |
| SQ | SEQUENCE | 238 | AA; | 25389 | MW; | AE799B80E8393023 | CRC64; |
| SQ | SEQUENCE | 247 | AA; | 26573 | MW; | AE987B9D32D58F93 | CRC64; |
| SQ | SEQUENCE | 238 | AA; | 25269 | MW; | 3BA22FF2EA32E4B5 | CRC64; |
| SQ | SEQUENCE | 246 | AA; | 26900 | MW; | 1EBE59D88BAB1715 | CRC64; |
| SQ | SEQUENCE | 237 | AA; | 25021 | MW; | 4072133E55022C76 | CRC64; |
| SQ | SEQUENCE | 248 | AA; | 24576 | MW; | 1A0EBA88C3E70294 | CRC64; |
| SQ | SEQUENCE | 231 | AA; | 24409 | MW; | A0A125CF7FC138C2 | CRC64; |
| SQ | SEQUENCE | 227 | AA; | 23308 | MW; | D5AC5E47B227B418 | CRC64; |
| SQ | SEQUENCE | 229 | AA; | 24591 | MW; | E83B83C5AD72FCE4 | CRC64; |
| SQ | SEQUENCE | 243 | AA; | 24946 | MW; | 261BF2614B4566B4 | CRC64; |
| SQ | SEQUENCE | 243 | AA; | 24654 | MW; | CADE6728CE05FF1D | CRC64; |
| SQ | SEQUENCE | 248 | AA; | 25872 | MW; | AC606B8998413305 | CRC64; |
| SQ | SEQUENCE | 245 | AA; | 26166 | MW; | E98FAF767BCAEB8F | CRC64; |
| SQ | SEQUENCE | 247 | AA; | 26309 | MW; | AD73E88531970324 | CRC64; |
| SQ | SEQUENCE | 242 | AA; | 26180 | MW; | 08D2A834FB289080 | CRC64; |
| SQ | SEQUENCE | 243 | AA; | 25773 | MW; | DFA4B453FBDA777E | CRC64; |
| SQ | SEQUENCE | 249 | AA; | 27400 | MW; | 8FB98462CEDBEFC9 | CRC64; |
| SQ | SEQUENCE | 244 | AA; | 26317 | MW; | 0EB3B68E8706D52D | CRC64; |
| SQ | SEQUENCE | 237 | AA; | 25726 | MW; | 30D2DBAAC39080C2 | CRC64; |
| SQ | SEQUENCE | 249 | AA; | 27169 | MW; | 14F2F0B4F0C6B170 | CRC64; |
| SQ | SEQUENCE | 242 | AA; | 26201 | MW; | 3F4DE7CE80C4477C | CRC64; |
| SQ | SEQUENCE | 241 | AA; | 26282 | MW; | FE362D39CAEEB2F6 | CRC64; |
| SQ | SEQUENCE | 235 | AA; | 25232 | MW; | AB39A28C264A0604 | CRC64; |
| SQ | SEQUENCE | 247 | AA; | 26948 | MW; | DC4B647179DDD972 | CRC64; |
| SQ | SEQUENCE | 238 | AA; | 26071 | MW; | F2B8908085B8D062 | CRC64; |
| SQ | SEQUENCE | 228 | AA; | 24971 | MW; | 013E1B2B32EAE1FD | CRC64; |
| SQ | SEQUENCE | 223 | AA; | 24844 | MW; | C34EBE9455DD7DE9 | CRC64; |
| SQ | SEQUENCE | 242 | AA; | 25963 | MW; | 29A1FD2B55874DE0 | CRC64; |
| SQ | SEQUENCE | 245 | AA; | 26508 | MW; | 433DD289B4DC78E5 | CRC64; |
| SQ | SEQUENCE | 248 | AA; | 26067 | MW; | 1AEB8C3952E3863E | CRC64; |

TABLE 11
Header information for 48 Monoclonal antibody sequences used in the fold
recognition analysis. Sequences obtained from NCBI database.

>gi|576325|pdb|1VFA|B Chain B, Fv Fragment Of Mouse Monoclonal Antibody D1.3 (Ba
>gi|9438803|gb|AAB35976.2| anti-human apolipoprotein E Ig heavy chain variable r
>gi|481379|pir||S38563 Ig heavy chain V region (ASWS1) - mouse (fragment)
>gi|297575|emb|CAA79994.1| immunoglobulin variable region [Mus musculus domestic
>gi|4558136|pdb|1BVK|B Chain B, Humanized Anti-Lysozyme Fv Complexed With Lysozy
>gi|602315|gb|AAA57212.1| Igh [Mus musculus]
>gi|384300|prf||1905384A anti-neuropeptide Y antibody variable region: SUBUNIT = he
>gi|477473|pir||A49049 Ig heavy chain V region (anti-idiotypic) - mouse
>gi|1098273|prf||2115359A anti-interleukin 5 antibody: SUBUNIT = heavy chain
>gi|400311|gb|AAA21370.1| anti-glycophorin A type N immunoglobulin heavy chain
>gi|12278342|gb|AAG49008.1| anti-human C3a receptor single-chain variable fragme
>gi|398538|gb|AAA21365.1| immunoglobulin heavy chain [Mus musculus]
>gi|27818733|gb|AAO23366.1| immunoglobulin heavy chain variable region [Mus musc
>gi|19697130|gb|AAL92937.1| BW2 25-32 immunoglobulin heavy chain [Mus musculus]
>gi|4090716|gb|AAC98861.1| IgG heavy chain variable region [Mus musculus]
>gi|288707|emb|CAA80021.1| immunoglobulin variable region [Mus musculus domestic
>gi|423378|pir||S32786 Ig heavy chain (anti-biotin) - mouse
>gi|90805|pir||PQ0266 Ig heavy chain V region (MC1) - mouse (fragment)
>gi|1553000|gb|AAC52875.1| metal binding monoclonal antibody 2A81G5 heavy chain
>gi|5690311|gb|AAD47031.1| immunoglobulin heavy chain variable region [Mus muscu
>gi|1518305|emb|CAA62390.1| antibody heavy chain variable region [Mus musculus]
>gi|288816|emb|CAA80065.1| immunoglobulin variable region [Mus musculus domestic
>gi|5853164|gb|AAD54343.1| immunoglobulin heavy chain variable region [Mus muscu
>gi|90770|pir|PL0087 Ig heavy chain V region (E3) - mouse
>gi|30525708|gb|AAP32218.1| DR5 monoclonal antibody 1 heavy chain variable regio
>gi|20797183|emb|CAD30987.1| anti-human CD28 specific monoclonal antibody 9.3 [M
>gi|913649|gb|AAB33456.1| anti-carcinoembryonic antigen CEA monoclonal antibody
>gi|602709|gb|AAA61339.1| anti-DNA autoantibody
>gi|2677627|emb|CAA74659.1| immunoglobulin heavy chain, variable region [Mus mus
>gi|1872285|gb|AAB49081.1| anti-DNA immunoglobulin heavy chain IgG [Mus musculus
>gi|9944969|gb|AAG03052.1|AF287274_1 immunoglobulin gamma heavy chain variable r
>gi|1334266|emb|CAA53609.1| immunoglobulin V-region heavy chain [Mus musculus]
>gi|4426895|gb|AAD20593.1| monoclonal antibody 5C3 heavy chain V region [Mus mus
>gi|15281300|dbj|BAB63404.1| anti-CD98 monoclonal antibody HBJ127 heavy chain va
>gi|3328075|gb|AAC26769.1| monoclonal anti-DNA IgM heavy chain variable region [
>gi|32263955|gb|AAP78497.1| mAb immunoglobulin heavy chain variable region [Mus
>gi|5822541|pdb|43CA|B Chain B, Crystallographic Structure Of The Esterolytic An
>gi|2253324|gb|AAB62902.1| IgMk heavy chain variable region [Mus musculus]
>gi|631638|pir||PL0198 anti-DNA autoantibody BV16-13, heavy chain V region - mou
>gi|7414589|emb|CAB85940.1| immunoglobulin heavy chain variable region [Rattus n
>gi|27818663|gb|AAO23314.1| immunoglobulin heavy chain variable region [Mus musc
>gi|4379205|emb|CAA41886.1| unnamed protein product [Mus musculus]
>gi|10185111|emb|CAC08525.1| immunoglobulin heavy chain [Mus musculus]
>gi|15150239|gb|AAK85364.1| moderate affinity anti-nucleosome binding monoclonal
>gi|1262255|gb|AAA96770.1| IgG anti-nucleosome heavy chain variable region
>gi|11128000|gb|AAG31175.1|AF316987_1 rearranged immunoglobulin heavy chain vari
>gi|6433982|emb|CAB60627.1| immunoglobulin heavy chain variable region [Rattus n
>gi|4633314|gb|AAD26713.1| immunoglobulin heavy chain VHQ52-JH1 region [Mus musc

TABLE 12
Header information for 64 Amido transferase sequences used in the fold
recognition analysis. Sequences obtained from NCBI database.

>gi|16761006|ref|NP_456623.1| amidotransferase [Salmonella enterica subsp. enter
>gi|16121818|ref|NP_405131.1| amidotransferase [Yersinia pestis]
>gi|28897915|ref|NP_797520.1| amidotransferase HisH [Vibrio parahaemolyticus RIM
>gi|15641149|ref|NP_230781.1| amidotransferase HisH [Vibrio cholerae]
>gi|15616724|ref|NP_239936.1| amidotransferase hisH [Buchnera aphidicola str. AP
>gi|21672388|ref|NP_660455.1| amidotransferase [Buchnera aphidicola str. Sg (Sch
>gi|33519917|ref|NP_878749.1| glutamine amidotransferase, subunit with HisF [Can
>gi|27904601|ref|NP_777727.1| amidotransferase HisH [Buchnera aphidicola (Baizon
>gi|15792905|ref|NP_282728.1| amidotransferase HisH [Campylobacter jejuni]
>gi|29346790|ref|NP_810293.1| imidazole glycerol phosphate synthase subunit hisH
>gi|23136279|ref|ZP_00118003.1| hypothetical protein [Cytophaga hutchinsonii]
>gi|21231260|ref|NP_637177.1| amidotransferase [Xanthomonas campestris pv. campe
>gi|15598348|ref|NP_251842.1| glutamine amidotransferase [Pseudomonas aeruginosa
>gi|20089791|ref|NP_615866.1| imidazoleglycerol-phosphate synthase, subunit H [M
>gi|12230929|sp|Q57929|HIS5_METJA Imidazole glycerol phosphate synthase subunit
>gi|28199150|ref|NP_779464.1| amidotransferase [Xylella fastidiosa Temecula1]
>gi|15679521|ref|NP_276638.1| imidazoleglycerol-phosphate synthase [Methanotherm
>gi|30019557|ref|NP_831188.1| Amidotransferase hisH [Bacillus cereus ATCC 14579]
>gi|21399323|ref|NP_655308.1| GATase, Glutamine amidotransferase class-I [Bacill
>gi|15559183|gb|AAK58487.1| unknown [Campylobacter jejuni]
>gi|18978033|ref|NP_579390.1| glutamine amidotransferase [Pyrococcus furiosus DS
>gi|21673311|ref|NP_661376.1| amidotransferase HisH [Chlorobium tepidum TLS]
>gi|23020794|ref|ZP_00060489.1| hypothetical protein [Clostridium thermocellum A
>gi|15673194|ref|NP_267368.1| amidotransferase [Lactococcus lactis subsp. lactis
>gi|23098004|ref|NP_691470.1| amidotransferase [Oceanobacillus iheyensis HTE831]
>gi|15894226|ref|NP_347575.1| Glutamine amidotransferase [Clostridium acetobutyl
>gi|23023420|ref|ZP_00062656.1| hypothetical protein [Leuconostoc mesenteroides
>gi|22299079|ref|NP_682326.1| amidotransferase [Thermosynechococcus elongatus BP
>gi|32475855|ref|NP_868849.1| amidotransferase hisH [Pirellula sp.]
>gi|20559824|gb|AAM27599.1|AF498403_18 ORF_18; similar to Glutamine amidotransfe
>gi|20808525|ref|NP_623696.1| Glutamine amidotransferase [Thermoanaerobacter ten
>gi|11499846|ref|NP_071090.1| imidazoleglycerol-phosphate synthase, subunit H (h
>gi|15925665|ref|NP_373199.1| amidotransferase hisH [Staphylococcus aureus subsp
>gi|22406016|ref|ZP_00000875.1| hypothetical protein [Ferroplasma acidarmanus]
>gi|27467192|ref|NP_763829.1| amidotransferase hisH [Staphylococcus epidermidis
>gi|24379687|ref|NP_721642.1| putative glutamine amidotransferase HisH [Streptoc
>gi|12229845|sp|Q9S4H8|HI52_LEPIN Imidazole glycerol phosphate synthase subunit
>gi|16802608|ref|NP_464093.1| similar to amidotransferases [Listens monocytogen
>gi|15827646|ref|NP_301909.1| glutamine amidotransferase [Mycobacterium leprae]
>gi|16799649|ref|NP_469917.1| similar to amidotransferases [Listeria innocua]
>gi|15841055|ref|NP_336092.1| amidotransferase HisH [Mycobacterium tuberculosis
>gi|12229850|sp|Q9ZGM1|HIS5_LEPBO Imidazole glycerol phosphate synthase subunit
>gi|32261065|emb|CAE00216.1| glutamine amidotransferase [Rhizobium leguminosarum
>gi|20094946|ref|NP_614793.1| Glutamine amidotransferase [Methanopyrus kandleri
>gi|28379092|ref|NP_785984.1| amidotransferase [Lactobacillus plantarum WCFS1]
>gi|33861616|ref|NP_893177.1| Glutamine amidotransferase class-I [Prochlorococcu
>gi|18312307|ref|NP_558974.1| amidotransferase (hisH) [Pyrobaculum aerophilum]
>gi|27065167|pdb|1KA9|H Chain H, Imidazole Glycerol Phosphate Synthase
>gi|30468160|ref|NP_849047.1| amidotransferase hisH [Cyanidioschyzon merolae]
>gi|15897516|ref|NP_342121.1| Amidotransferase hisH (hisH) [Sulfolobus solfatari
>gi|20138285|sp|Q970Y7| HIS5_SULTO Imidazole glycerol phosphate synthase subunit
>gi|14602143|ref|NP_148691.1| anthranilate synthase component II [Aeropyrum pern
>gi|15901645|ref|NP_346249.1| anthranilate synthase component II [Streptococcus
>gi|15603328|ref|NP_246402.1| TrpG [Pasteurella multocida]
>gi|18313360|ref|NP_560027.1| anthranilate synthase component II [Pyrobaculum ae
>gi|22997331|ref|ZP_00041564.1| hypothetical protein [Xylella fastidiosa Ann-1]
>gi|13541854|ref|NP_111542.1| Anthranilate synthase component II [Thermoplasma v
>gi|15896410|ref|NP_349759.1| Para-aminobenzoate synthase component II [Clostrid
>gi|29345941|ref|NP_809444.1| anthranilate synthase component II [Bacteroides th
>gi|11499195|ref|NP_070431.1| anthranilate synthase component II (trpG) [Archaeo
>gi|16801952|ref|NP_472220.1| similar to glutamine amidotransferase [Listeria in
>gi|15921491|ref|NP_377160.1| 193aa long hypothetical anthranilate synthase comp
>gi|17227765|ref|NP_484313.1| anthranilate synthase component II [Nostoc sp. PCC
>gi|15673451|ref|NP_267625.1| anthranilate synthase component II [actococcus la

TABLE 13
Header information for 30 sequences randomly selected from each of the six
protein families. These 30 sequences are used to test the fold recognition
using protein signals in Example 2.

| | | | | |
|---|---|---|---|---|
| SQ | SEQUENCE | 146 AA; | 15774 MW; | D8F96C641E7EF653 CRC64; |
| SQ | SEQUENCE | 149 AA; | 16338 MW; | C5CC39B31C4CE346 CRC64; |
| SQ | SEQUENCE | 141 AA; | 15122 MW; | E4EE4DE6485050F6 CRC64; |
| SQ | SEQUENCE | 146 AA; | 16205 MW; | 1045D760101D8EC1 CRC64; |
| SQ | SEQUENCE | 147 AA; | 17376 MW; | CDC1DFA489AEE980 CRC64; |
| SQ | SEQUENCE | 151 AA; | 17132 MW; | FDAE65E7ADD6BA47 CRC64; |
| SQ | SEQUENCE | 148 AA; | 16497 MW; | 1BAE0FB352E22602 CRC64; |
| SQ | SEQUENCE | 159 AA; | 17896 MW; | DD081C9CB5946277 CRC64; |
| SQ | SEQUENCE | 142 AA; | 16608 MW; | 740915F02FA76040 CRC64; |
| SQ | SEQUENCE | 150 AA; | 16361 MW; | 5290E082A7C79349 CRC64; |
| SQ | SEQUENCE | 106 AA; | 11307 MW; | 7B905E1C85AFB234 CRC64; |
| SQ | SEQUENCE | 110 AA; | 11988 MW; | C82608E316F91744 CRC64; |
| SQ | SEQUENCE | 112 AA; | 11712 MW; | E8F93797CECBC77C CRC64; |
| SQ | SEQUENCE | 104 AA; | 11672 MW; | E3DA258BEACEE9BE CRC64; |
| SQ | SEQUENCE | 110 AA; | 12166 MW; | 37AC78B37E8E6E45 CRC64; |
| SQ | SEQUENCE | 248 AA; | 26693 MW; | 8D73295CA82B62E8 CRC64; |
| SQ | SEQUENCE | 246 AA; | 26203 MW; | CEF8C97AAC2D07AD CRC64; |
| SQ | SEQUENCE | 245 AA; | 25755 MW; | 678016446FF5FEB5 CRC64; |
| SQ | SEQUENCE | 248 AA; | 26417 MW; | AEE31CC449CFFD4D CRC64; |
| SQ | SEQUENCE | 246 AA; | 26170 MW; | E9E5A1DE2391BBBB CRC64; |

>gi|110154|pir||S20809 Ig heavy chain V region (hybridoma C8) - mouse
>gi|5734452|emb|CAB52694.1| immunoglobulin heavy chain variable region [Mus musc
>gi|110132|pir||D30560 Ig heavy chain V region (36.1.2D) - mouse (fragment)
>gi|24571379|gb|AAN62970.1| immunoglobulin heavy chain variable region [Mus musc
>gi|24571304|gb|AAN62944.1| immunoglobulin heavy chain variable region [Mus musc
>gi|15603067|ref|NP_246139.1| HisH [Pasteurella multocida]
>gi|15643796|ref|NP_228844.1| amidotransferase [Thermotoga maritima]
>gi|421728|pir||B40635 anthranilate synthase (EC 4.1.3.27) component II [validat
>gi|16272420|ref|NP_438633.1| amidotransferase [Haemophilus influenzae Rd]
>gi|16126140|ref|NP_420704.1| glutamine amidotransferase, class I [Caulobacter c

TABLE 14
Significant signals, generated using class 1 amino acids,
identified from the collection of protein sequence in Table 3.

| | Expected | Observed | | Signal | | Signal |
|---|---|---|---|---|---|---|
| Signal ID | #occurrences | #occurrences | Sequence x² | frequency | Signal | strength |
| 22 | 1589.49 | 2615 | 661.642 | 1.65 | 000000000 | 0 |
| 49 | 1045.55 | 1329 | 76.843 | 1.27 | 000000010 | 1 |
| 85 | 84.70 | 133 | 27.546 | 1.57 | 101011111 | 7 |
| 94 | 1045.55 | 1283 | 53.925 | 1.23 | 000100000 | 1 |
| 152 | 84.70 | 135 | 29.874 | 1.59 | 110111011 | 7 |
| 163 | 452.40 | 356 | 20.541 | 0.79 | 010001100 | 3 |
| 258 | 55.71 | 114 | 60.978 | 2.05 | 111011111 | 8 |
| 268 | 297.58 | 223 | 18.693 | 0.75 | 101100001 | 4 |
| 281 | 55.71 | 143 | 136.752 | 2.57 | 110111111 | 8 |
| 299 | 84.70 | 139 | 34.815 | 1.64 | 110011111 | 7 |
| 334 | 84.70 | 140 | 36.109 | 1.65 | 011011111 | 7 |
| 445 | 55.71 | 113 | 58.904 | 2.03 | 111111101 | 8 |
| 469 | 128.76 | 188 | 27.254 | 1.46 | 111111000 | 6 |
| 510 | 84.70 | 157 | 61.720 | 1.85 | 011111110 | 7 |

TABLE 15
Significant signals, generated using class 2 amino acids,
identified from the collection of protein sequences in Table 3..

| Signal | Expected | Observed | Sequence | Signal | | Signal |
|---|---|---|---|---|---|---|
| ID | #occurrences | #occurrences | x² | frequency | Signal | strength |
| 42 | 950.69 | 1206 | 68.562 | 1.27 | 001000100 | 2 |
| 62 | 488.42 | 778 | 171.689 | 1.59 | 001001100 | 3 |
| 69 | 250.93 | 458 | 170.885 | 1.83 | 100110010 | 4 |
| 112 | 1850.49 | 1411 | 104.380 | 0.76 | 100000000 | 1 |
| 113 | 3601.92 | 1998 | 714.220 | 0.55 | 000000000 | 0 |
| 149 | 250.93 | 408 | 98.324 | 1.63 | 011001100 | 4 |
| 183 | 66.23 | 28 | 22.067 | 0.42 | 111010101 | 6 |
| 185 | 128.91 | 77 | 20.906 | 0.60 | 101010101 | 5 |
| 231 | 250.93 | 395 | 82.722 | 1.57 | 001101100 | 4 |
| 375 | 66.23 | 23 | 28.217 | 0.35 | 011011110 | 6 |
| 397 | 66.23 | 26 | 24.437 | 0.39 | 110001111 | 6 |
| 407 | 34.03 | 9 | 18.406 | 0.26 | 011101111 | 7 |

TABLE 16
Class 2 examples of centroid-signal correlations

| | | | Centroid | Centroid |
|---|---|---|---|---|
| | | | abundance in | abundance when |
| Signal# | Signal | Centroid | training set (%) | signal present (%) |
| 28 | 010011000 | alpha | 22.8 | 51.0 |
| | | helix | | |
| 290 | 110000111 | beta | 1.8 | 13.8 |
| | | hairpin | | |
| 66 | 100001001 | extended | 3.3 | 27.0 |
| | | helix | | |
| 358 | 011110011 | beta | 28.9 | 64.7 |
| | | strand | | |

TABLE 17
Associated statistics of signal-secondary structure correlations
of Table 16.

| | #Occurrences | Sequence | Local structure |
|---|---|---|---|
| Signal# | in training set | x² | x² |
| 28 | 624 | 37.6 | 363.7 |
| 290 | 116 | 1.3 | 178.8 |
| 66 | 647 | 51.5 | 1468.1 |
| 358 | 34 | 15.7 | 105.9 |

1. A computer-implemented method of analyzing a sequence of amino acids, comprising;
(a) designating each amino acid within the sequence with a symbol, wherein an amino acid is designated a first symbol if it is a member of a predetermined set of amino acids, and a second symbol different from the first symbol if the amino acid is not a member of the predetermined set, thereby producing a sequence of symbols;
(b) determining which signals of the symbols are present in the sequence of symbols, wherein a signal is a window of the sequence of symbols consisting of a predefined number of contiguous symbols;
wherein the sequence of amino acids is analyzed from the identity of the signals present in the sequence of symbols.

2. The method of claim 1, wherein the window consists of 5-15 contiguous symbols.

3. The method of claim 1, wherein the window consists of 9 contiguous symbols.

4. The method of claim 1, wherein the predetermined set of amino acids consists of 4-10 amino acids, and at least 4 are selected from the group consisting of A, R, Q, E, L, K and M.

5. The method of claim 4, wherein the predetermined set of amino acids consists of A, R, Q, E, L, K and M.

6. The method of claim 1, wherein the predetermined set of amino acids consists of 4-10 amino acids, and at least 4 are selected from the group consisting of C, I, L, M, F, W, Y, and V.

7. The method of claim 6, wherein the predetermined set of amino acids consists of C, I, L, M, F, W, Y, and V.

8. The method of claim 1, further comprising transforming the sequence of symbols into a sequence of signal designations, wherein different designations are used to represent different signals in the sequence of symbols.

9. The method of claim 1, wherein an amino acid is designated with a first type of second symbol if it is part of a second predetermined set of amino acids, and a second type of second symbol if it is not part of the second set of amino acids.

10. The method of claim 1, wherein the signals present in the sequence of symbols are assigned grades according to the probability that the observed frequency of a signal in a collection of proteins in which each amino acid has been designated with a symbol occurs by chance, wherein the grade increases with decreasing probability.

11. The method of claim 10, wherein the signals are classified as significant or not significant signals depending whether the grade exceeds a threshold.

12. The method of claim 11, wherein the threshold is a ²>8 that the observed frequency of the signal in the collection of proteins does not occur by chance.

13. The method of claim 12, further comprising determining the number and identity of significant signals in the amino acid sequence.

14. The method of claim 13, wherein the sequence of amino acids is a theoretical amino acid sequence, and the method further comprises determining the probability that the theoretical amino acid sequence is an actual protein by comparing the expected number of significant signals in the theoretical amino acid sequence to the actual number of significant signals in the theoretical amino acid sequence.

15. The method of claim 14, wherein the theoretical amino acid sequence is designated as an actual protein sequence if the probability that the observed significant signals in the sequence arose by chance is 10⁻¹ or less.

16. The method of claim 1, wherein the sequence of amino acids is from a known protein.

17. The method of claim 1, wherein the sequence of amino acids is from a putative protein.

18. The method of claim 1, further comprising repeating steps (a) and (b) for a second sequence of amino acids and aligning the sequences of symbols produced from the first and second sequences of amino acids for maximum conservation of significant signals.

19. The method of claim 11, further comprising predicting the secondary structure of a segment of a protein located within the sequence of amino acids from the identity of significant signals.

20. The method of claim 19, wherein the secondary structure is selected from the group consisting of an alpha helix, beta strand, beta turn, turn+beta, helix+turn, helix cap, extended helix, Gly/Pro twist, beta+turn, helix-hairpin, beta cap, helix hairpin, beta hairpin, contorted helix, turn, helix+turn II and helix turn.

21. The method of claim 1, further comprising inputting the sequence of amino acids into the computer.

22. The method of claim 21, wherein the sequence of amino acids is input by transfer of data from a database.

23. The method of claim 1, further comprising outputting the identity of signals present in the sequence of symbols.

24. The method of claim 23, wherein the signals are output in an order corresponding to the order of amino acids in the sequence of amino acids.

25. The method of claim 1, further comprising providing user input of the predefined number of contiguous symbols of the window.

26. The method of claim 10, further comprising calculating the probability that the observed frequency of a signal in the collection of proteins in which each amino acid has been designated with a symbol occurs by chance.

27. The method of claim 1, wherein step (b) determines the identity of L-(P-1) signals within the sequence of amino acids, where L is length of the sequence of amino acids and P is the predefined number of contiguous symbols in the window.

28. The method of claim 1, further comprising assigning the determined signals designations, a different designation being used for each unique signal.

29. The method of claim 1, further comprising analyzing the sequence of amino acids from the identity of the signals.

30. A computer implemented method of identifying a set of amino acids useful for the analysis of proteins, comprising
(a) designating each amino acid within each of a collection of proteins with a symbol, wherein an amino acid is designated a first symbol if it is a member of a first test set of amino acids, and a second symbol different from the first symbol if the amino acid is not a member of the test set, thereby producing a collection of sequences of symbols;
(b) determining the number of occurrences of different signals of the symbols in the collection of sequences of symbols, wherein a signal is a window of the sequence of symbols consisting of a predefined number of contiguous symbols; and
(c) determining the probability that the distribution of the number of signals of each signal strength occurs by chance, wherein the lower the probability the more useful the test set of amino acids is for protein analysis.

31. The method of claim 30, further comprising repeating steps (a), (b) and (c) for a second test set of amino acids.

32. The method of claim 31, wherein the second test set differs from the first test set by the addition, deletion, or substitution of an amino acid from the first test set.

33. The method of claim 32, further comprising repeating steps (a), (b) and (c) for each possible unique set of amino acids consisting of 4-10 amino acids.

34. The method of claim 1, further comprising comparing the position and identity of each signal present in the sequence of symbols to a conserved signal pattern present in a family of proteins.

35. A computer-implemented method of predicting the fold of a query protein comprising;
(a) designating each amino acid within a family of protein sequences with a symbol, wherein an amino acid is designated a first symbol if it is a member of a predetermined set of amino acids, and a second symbol different from the first symbol if the amino acid is not a member of the set, thereby producing a plurality of sequences of symbols;
(b) determining which signals of the symbols are present in the sequences of symbols, wherein a signal is a window of the sequence of symbols consisting of a predefined number of contiguous symbols;
(c) determining a conserved signal pattern between members of the family;
(d) analyzing a query protein to identify a signal pattern;
(e) determining if the query protein's signal pattern exceeds a threshold of similarity to the conserved signal pattern; and
(f) if the signal pattern of the query exceeds the threshold, designating the query as having the fold of the family.

36. The method of claim 35, further comprising comparing the query protein's signal pattern to conserved signal patterns in an additional protein family.

37. The method of claim 36, wherein the family is selected from the list consisting of globins, lysozymes, thioredoxins, trypsins, monoclonal antibodies, and amido transferases.

38. The method of claim 35, wherein the conserved signal pattern includes a signal present in Table 14.

39. The method of claim 35, wherein the conserved signal pattern includes a signal present in Table 15.

40. The method of claim 5, wherein at least one signal present in the sequence of symbols is present in Table 14.

41. The method of claim 7, wherein at least one signal present in the sequence of symbols is present in Table 14.

42. The method of claim 5, wherein at least one signal present in the sequence of symbols is present in Table 15.

43. The method of claim 7, wherein at least one signal present in the sequence of symbols is present in Table 15.

44. The method of claim 14, wherein at least one signal present in the sequence of symbols is present in Table 14.

45. The method of claim 14, wherein at least one signal present in the sequence of symbols is present in Table 15.

46. A computer program product stored on a computer readable media for analyzing a sequence of amino acids, the program product comprising;
(a) code for designating each amino acid within the sequence with a symbol, wherein an amino acid is designated a first symbol if it is a member of a predetermined set of amino acids, and a second symbol different from the first symbol if the amino acid is not a member of the set, thereby producing a sequence of symbols; and
(b) code for determining which signals of the symbols are present in the sequence of symbols, wherein a signal is a window of the sequence of symbols consisting of a predefined number of contiguous symbols, wherein the sequence of amino acids is analyzed from the identity of the signals present in the sequence of symbols.

47. A computer program product stored on a computer readable media for identifying a set of amino acids useful for the analysis of proteins, the program product comprising:
(a) code for designating each amino acid within each of a collection of proteins with a symbol, wherein an amino acid is designated a first symbol if it is a member of a first test set of amino acids, and a second symbol different from the first symbol if the amino acid is not a member of the test set, thereby producing a collection of sequences of symbols;
(b) code for determining the number of occurrences of different signals of the symbols in the collection of sequences of symbols, wherein a signal is a window of the sequence of symbols consisting of a predefined number of contiguous symbols; and
(c) code for determining the probability that the distribution of the number of signals of each signal strength occurs by chance, wherein the lower the probability the more useful the test set of amino acids is for protein analysis.

48. A computer program product stored on a computer readable media for predicting the fold of a query protein, the program product comprising:
(a) code for designating each amino acid within a family of protein sequences with a symbol, wherein an amino acid is designated a first symbol if it is a member of a predetermined set of amino acids, and a second symbol different from the first symbol if the amino acid is not a member of the set, thereby producing a plurality of sequences of symbols;
(b) code for determining which signals of the symbols are present in the sequences of symbols, wherein a signal is a window of the sequence of symbols consisting of a predefined number of contiguous symbols;
(c) code for determining a conserved signal pattern between members of the family;
(d) code for analyzing a query protein to identify a signal pattern;
(e) code for determining if the query protein's signal pattern exceeds a threshold of similarity to the conserved signal pattern; and
(f) code for designating the query as having the fold of the family if the signal pattern of the query exceeds the threshold.

49. A computer program product stored on a computer readable media for identifying a coding region of a nucleotide sequence, the program product comprising:
(a) code for translating all possible reading frames of a nucleotide sequence into theoretical protein sequences;
(b) code for designating each amino acid within the theoretical protein sequences with a symbol, wherein an amino acid is designated a first symbol if it is a member of a first predetermined set of amino acids, and a second symbol different from the first symbol if the amino acid is not a member of the predetermined set, thereby producing a collection of sequences of symbols;
(c) code for determining the number of significant signals in each reading frame of the nucleotide sequence; and
(d) code for determining an expected number of significant signals in each reading frame of the nucleotide sequence.

50. A system for analyzing a sequence of amino acids, comprising:
(a) a processor; and
(b) a memory coupled to the processor configured to store a plurality of instructions which when executed by the processor cause the processor to:
(i) designate each amino acid within the sequence with a symbol, wherein an amino acid is designated a first symbol if it is a member of a predetermined set of amino acids, and a second symbol different from the first symbol if the amino acid is not a member of the set, thereby producing a sequence of symbols;
(ii) determine which signals of the symbols are present in the sequence of symbols, wherein a signal is a window of the sequence of symbols consisting of a predefined number of contiguous symbols, wherein the sequence of amino acids is analyzed from the identity of the signals present in the sequence of symbols.

51. A system for identifying a set of amino acids useful for the analysis of proteins comprising:
(a) a processor; and
(b) a memory coupled to the processor configured to store a plurality of instructions which when executed by the processor cause the processor to:
(i) designate each amino acid within each of a collection of proteins with a symbol, wherein an amino acid is designated a first symbol if it is a member of a first test set of amino acids, and a second symbol different from the first symbol if the amino acid is not a member of the test set, thereby producing a collection of sequences of symbols;
(ii) determine the number of occurrences of different signals of the symbols in the collection of sequences of symbols, wherein a signal is a window of the sequence of symbols consisting of a predefined number of contiguous symbols; and
(iii) determine the probability that the distribution of the number of signals of each signal strength occurs by chance, wherein the lower the probability the more useful the test set of amino acids is for protein analysis.

52. A system for predicting the fold of a query protein comprising:
(a) a memory;
(b) a system bus;
(c) a processor operatively disposed to:
(i) designate each amino acid within a family of protein sequences with a symbol, wherein an amino acid is designated a first symbol if it is a member of a predetermined set of amino acids, and a second symbol different from the first symbol if the amino acid is not a member of the set, thereby producing a plurality of sequences of symbols;
(ii) determine which signals of the symbols are present in the sequences of symbols, wherein a signal is a window of the sequence of symbols consisting of a predefined number of contiguous symbols;
(iii) determine a conserved signal pattern between members of the family;
(iv) analyze a query protein to identify a signal pattern;
(v) determine if the query protein's signal pattern exceeds a threshold of similarity to the conserved signal pattern; and
(vi) designate the query as having the fold of the family if the signal pattern of the query exceeds the threshold.

53. A system for identifying a coding region of a nucleotide sequence comprising:
(a) a memory;
(b) a system bus;
(c) a processor operatively disposed to:
(i) translate all possible reading frames of a nucleotide sequence into theoretical protein sequences;
(ii) designate each amino acid within the theoretical protein sequences with a symbol, wherein an amino acid is designated a first symbol if it is a member of a first predetermined set of amino acids, and a second symbol different from the first symbol if the amino acid is not a member of the predetermined set, thereby producing a collection of sequences of symbols;
(iii) determine the number of significant signals in each reading frame of the nucleotide sequence; and
(iv) determine an expected number of significant signals in each reading frame of the nucleotide sequence.


SEQUENCE LISTING (6 sequences)

SEQ ID NO: 1 - protein, 9 aa, Artificial
  Chemically synthesized
ANISVYYEM

SEQ ID NO: 2 - protein, 9 aa, Artificial
  Chemically synthesized
TSFNFWMGV

SEQ ID NO: 3 - protein, 9 aa, Artificial
  Chemically synthesized
SGCGLILNC

SEQ ID NO: 4 - protein, 11 aa, Artificial
  Chemically synthesized
PAGEQEAFPPN

SEQ ID NO: 5 - protein, 7 aa, Artificial
  Chemically synthesized
ARQELKM

SEQ ID NO: 6 - protein, 8 aa, Artificial
  Chemically synthesized
CILMFWYV